(19) United States
(12) Patent Application Publication
(10) Pub. No.: US 2025/0353857 A1
(43) Pub. Date: Nov. 20, 2025

(54) COMPOUND ACTIVE AS INHIBITOR OF COLONY STIMULATION FACTOR-1 RECEPTOR (CSF-1 R)

(71) Applicant: Norwegian University of Science and Technology (NTNU), Trondheim (NO)

(72) Inventors: Thomas Ihle AARHUS, Trondheim (NO); Eirik SUNDBY, Trondheim (NO); Bård Helge HOFF, Trondheim (NO); Frithjof BJØRNSTAD, Trondheim (NO); Geir BJØRKØY, Trondheim (NO)

(21) Appl. No.: 19/120,429

(22) PCT Filed: Oct. 13, 2023
(86) PCT No.: PCT/EP2023/078556

§ 371 Date: Apr. 11, 2025

(30) Foreign Application Priority Data
Oct. 13, 2022 (GB) 2215117.9

(51) Int. Cl. C07D 487/04; A61K 31/52; A61K 31/5377; A61K 31/675; C07D 473/34; C07D 495/04; C07F 9/6561

(52) U.S. Cl. CPC C07D 487/04; A61K 31/52; A61K 31/5377; A61K 31/675; C07D 473/34; C07D 495/04; C07F 9/6561

(57) ABSTRACT

The present invention relates to new pyrrolopyrimidines and purines which surprisingly act as CSF-1R inhibitors, to processes for making these compounds, and to uses thereof.

### SUMMARY OF THE INVENTION

The present invention relates to certain compounds, in particular new pyrrolopyrimidines and purines. These new compounds have been found to be inhibitors of CSF-1R (Colony stimulation factor-1 receptor), and as a result offer potential in the treatment of a number of diseases, such as cancers, bone disorders, neurological diseases, inflammatory disorders and eye diseases. The invention also relates to pharmaceutical compositions comprising said compounds, to the compounds for use in the treatment of various diseases, to methods of treating diseases by administration of said compounds, to the use of the compounds in the manufacture of a medicament for the treatment or prevention of various diseases, and to processes for the formation of said compounds.

### BACKGROUND TO THE INVENTION

CSF-1R (Colony stimulating factor-1 receptor) has emerged as an important pharmaceutical target as it plays an important role in a number of diseases (J. Med. Chem. 2018, 61, 13, 5450-5466, Journal for Immuno Therapy of Cancer (2017) 5:53, Biomedicine & Pharmacotherapy 103 (2018) 662-679). Colony stimulating factor-1 (CSF-1) is a common proinflammatory cytokine responsible for various disorders, which acts by binding to the receptor CSF-1R. CSF-1R or its activator the cytokine colony stimulation factor-1 (CSF1) is over expressed in many disorders and consequently is an important target. A number of inhibitors have been investigated, such as ABT-869, Imatinib, AG013736, JNJ-40346527, PLX3397, DCC-3014 and Ki20227. A few candidates have started clinical trials, but many side effects have been noted. The successful targeting of CSF-1R is therefore of importance to the pharmaceutical industry, and given its role in a number of diseases, even offers the possibility of multifunctional therapy (Biomedicine & Pharmacotherapy 103 (2018) 662-679).

Upon binding of CSF1 or IL-34 (interleukin 34) to the extracellular domain of CSF1R, also called M-CSFR (Macrophage colony-stimulating factor receptor) or CD115 (Cluster of Differentiation 115), the cell-surface receptor undergoes oligomerization and subsequent phosphorylation of tyrosine residues in the cytoplasmic kinase domain activating signal transduction. The kinase receptor mediates signalling responsible for the survival, function, proliferation and differentiation of cells in the myeloid lineage such as monocytes, macrophages, microglia and osteoclasts. (see Chitu, V.; Caescu, C. I.; R., S.; Lennartsson, J.; Rönnstrand, L.; Heldin, C. H., The PDGFR Receptor family, In: Receptor Tyrosine Kinases: Family and Subfamilies. Springer International Publishing, Switzerland: 2015; p 375-525)

The present group previously reported in WO2015/000959 certain new pyrrolo-, thieno-, and furo-[2,3-d]pyrimidine compounds and in particular, a series of such new 4-amino-6-aryl [2,3-d]pyrimidines. These compounds were found to be epidermal growth factor receptor tyrosine kinase (EGFR-TK) inhibitors and therefore offered potential in the treatment of cancer. The reported compounds all had a secondary amine group (NH) at the C-4 position of the bicyclic ring. An example of such a compound is the following, with the secondary amine in the C-4 position shown.

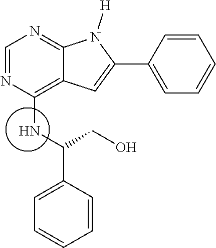

The present inventors have surprisingly found that substitution (e.g. methylation, ethylation etc.) at the C-4 nitrogen (in the case of pyrrolopyrimidines) can convert the compounds from EGFR inhibitors to CSF-1R inhibitors. This, unexpectedly, opens up a whole new field of therapeutic targeting for a number of diseases. It has been found that substitution at the exocyclic nitrogen typically blocks the EFGR activity and increases selectivity towards CSF-1R. It has also surprisingly been found that potent CSF-1R inhibition is achieved when the exocyclic nitrogen of other similar frameworks (e.g. C-6 nitrogen in the case of purines) is substituted (e.g. by methylation, ethylation etc.)

The inventors have also realised that other groups in the compounds reported herein are key to achieving high potency against CSF-1R, e.g. as they can affect solubility and/or achieve better matching with the shape of the receptor.

The present inventors have thus devised a class of compounds which surprisingly act as CSF-1R inhibitors, with structures typically based on pyrrolopyrimidines and purines. Given the known importance of targeting CSF-1R for a wide range of conditions, the present compounds represent a valuable new pharmaceutical class.

### SUMMARY OF INVENTION

In a first aspect, the invention concerns a compound of formula (I)

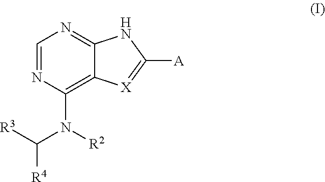

wherein:
X is N or CH;
A is a 5- or 6-membered hydrocarbyl or heterocyclic ring which may be optionally substituted with at least one R¹ group;
each R¹ is independently selected from halogen, hydroxyl, —OCF₃, —CF₃, —CF₂H, —OCF₂H, CH(CF₃)OH, —C₁-C₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, —C(CH₃)₂—CH₂OH, —C(OH)₂(C₁₋₆alkyl-OH), —C(OH)(C₁₋₆alkyl-OH)₂, —C(C₁₋₆alkyl-OH)₃, —C(CH₃)₂OH, —NH₂, NHR, —NR₂, —NO₂, —SO₂NH₂, —P(O)R₂, —SO₂R, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
each m is 0 or 1;
R² is methyl or ethyl, or deuterated or partially deuterated methyl or ethyl; or R² and R⁴ are linked so as to form a 5- or 6-membered ring;
R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or when R² and R⁴ are linked so as to form a 5- or 6-membered ring, R³ can also be H or a C₁-C₄ alkyl;
each R is C₁-C₆ alkyl;
R⁴ is hydrogen, deuterium, C₁-C₄ alkyl, C₁-C₄ alkyl substituted with a hydroxyl group, or ═O; or R² and R⁴ are linked so as to form a 5- or 6-membered ring;

or a pharmaceutically acceptable salt or solvate thereof.

In a further aspect of the invention, there is provided a pharmaceutical composition comprising a compound as defined herein and at least one excipient.

In a further aspect of the invention, there is provided a compound defined herein for use in the treatment or prevention of a bone disorder, neurological disease, inflammatory disorder, cancer, or eye disease.

In a further aspect of the invention, there is provided a method of treating or preventing a bone disorder, neurological disease, inflammatory disorder, cancer, or eye disease, comprising administering a compound as defined herein to a subject in need thereof, optionally in combination with other chemotherapy agents or radiotherapy when administered for treating or preventing cancer.

In a further aspect of the invention, there is provided the use of a compound as defined herein in the manufacture of a medicament for the treatment or prevention of a bone disorder, neurological disease, inflammatory disorder, cancer or eye disease.

In a further aspect of the invention, there is provided a process for the formation of a compound as defined herein, said process comprising the steps of:
reacting a compound of formula (IV):

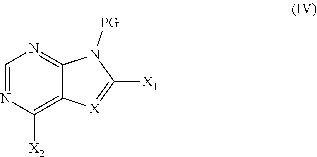

with
i) a compound of formula (RO)₂B-A or [A-BF₃]⁻ in the presence of a transition metal catalyst, and,
ii) a compound of formula:

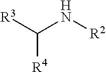

in either order (i.e. i) then ii) or ii) then i)); then
iii) removing the protecting group PG; wherein
X₁ and X₂ are halogen;
PG is a protecting group, preferably selected from -SEM, THP, BOC, Cbz, Fmoc, SO₂Ph, Ts, MOM, CO₂H;
R is OH, OMe, or (RO)₂ is pinacol (i.e. —O—C(CH₃)₂— C(CH₃)₂—O—) or MIDA ester (i.e. —O—CO—CH₂—N(CH₃)—CH₂—CO—O—),
A, X and R²-R⁴ are as defined above or below.

In a further aspect of the invention, there is provided a process for the formation of a compound as defined herein, said process comprising the steps of reacting a compound of formula

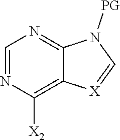

a) with a base followed by a cyclic ketone of a 6-membered hydrocarbyl or heterocyclic ring which may be optionally substituted with at least one R¹ group
b) with a compound of formula

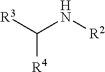

c) removing the protecting group PG;

wherein
X₂ is halogen;
PG is a protecting group, preferably selected from -SEM, THP, BOC, Cbz, Fmoc, SO₂Ph, MOM, CO₂H, Ts;
X and R¹-R⁴ are as defined above or below.

### Definitions

The following definitions may apply to all claimed compounds, unless otherwise indicated.

Any C₁₋₆ alkyl group is preferably a linear C₁₋₆ alkyl group. It is preferably a C₁₋₃ alkyl group, such as a linear C₁₋₃alkyl group, especially methyl, ethyl or n-propyl. However, any C₁₋₆ alkyl group can also be branched, e.g. —C(CH₃)₂—CH₂—, —C(CH₃)₂— etc.

When there are two or more ‘R’ groups or two or more ‘C₁₋₆-alkyl’ groups on a compound (e.g. —[C₁₋₆alkyl]ₘ—OCOC₁₋₆alkyl), then each R group or each C₁₋₆ group is independently selected from C₁-C₆ alkyl, i.e. the two or more R groups or the two or more C₁₋₆ alkyl groups may be different.

Any halogen or Hal, which stand for halogen/halide, is preferably Cl or F, ideally F. X₁ and X₂ are typically Cl and/or I.

The term hydrocarbyl ring is meant to include both aliphatic and aromatic hydrocarbyl rings. Aliphatic herein means non-aromatic, and can include alkyl, but also alkenyl, alkynyl etc. Aliphatic can include cyclic, acyclic, or a mixture of both.

Heterocyclic herein means both non-aromatic heterocycles and also heteroaryl groups. The heterocyclic groups may include one, two or three heteroatoms (e.g. N, O and/or or S), preferably one or two, most preferably one. Preferably, heterocyclic means herein a heterocycle with one heteroatom, typically selected from N, O and S.

By ‘optionally substituted’ is meant unsubstituted or substituted.

### DETAILED DESCRIPTION OF THE INVENTION

The invention relates to new compounds as CSF-1R inhibitors. The compounds preferably contain a substituted unit of formula (I):

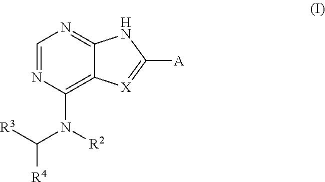

wherein:
X is N or CH;
A is a 5- or 6-membered hydrocarbyl or heterocyclic ring which may be optionally substituted with at least one R¹ group;
each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —P(O)R₂, —SO₂R, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
each m is 0 or 1;
R² is methyl or ethyl, or deuterated or partially deuterated methyl or ethyl; or R² and R⁴ are linked so as to form a 5- or 6-membered ring;
R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or when R² and R₄ are linked so as to form a 5- or 6-membered ring, R³ can also be H or a C₁-C₄ alkyl;
each R is C₁-C₆ alkyl;
R⁴ is hydrogen, deuterium, C₁-C₄ alkyl, C₁-C₄ alkyl substituted with a hydroxyl group, or ═O; or R² and R⁴ are linked so as to form a 5- or 6-membered ring;
or a pharmaceutically acceptable salt or solvate thereof.

The discussion of the various groups below are applicable to all embodiments and aspects of the invention, where technically viable, and are all combinable with one another.

X is N or CH.

In a particular embodiment, therefore the compound is selected from pyrrolopyrimidines, or purines.

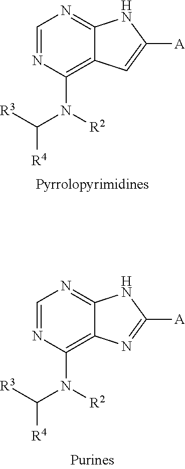

In all embodiments of the invention, A is a 5- or 6-membered hydrocarbyl or heterocyclic ring optionally substituted with at least one group R¹ group.

The 5- or 6-membered ring may be substituted with 0-5 R¹ groups, preferably 0-4 R¹ groups, more preferably 0-3 R¹ groups, more preferably 0-2 R¹ groups, more preferably 1-2 R¹ groups. Each R¹ group may be the same or different. The presence of a ring at the A position greatly increases CSF-1R potency. Simply changing A from a hydrogen to a phenyl ring can, in some instances, reduce the IC₅₀ from >200 nM to <5 nM. As will be discussed in more detail below, increasing the polarity of the A group also results in improved bioavailability and hence increased potency.

Each R¹ is independently selected from halogen, hydroxyl, —OCF₃, —CF₃, —CF₂H, —OCF₂H, —CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, —C(CH₃)₂—CH₂OH, —C(OH)₂(C₁₋₆alkyl-OH), —C(OH)(C₁₋₆alkyl-OH)₂, —C(C₁₋₆alkyl-OH)₃, —C(CH₃)₂OH, —NH₂, NHR, —NR₂, —NO₂, —SO₂NH₂, —P(O)R₂, —SO₂R, pyridyl (preferably ortho-pyridyl), cyclopropyl or cyclobutyl substituted with an —OH group, C₁-C₁₀ aliphatic hydrocarbyl group comprising at least one heteroatom selected from 0, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH. In the above, each R is a C₁-C₆ alkyl, and each m is 0 or 1.

Preferably, each R¹ is independently selected from halogen, hydroxyl, —CF₃, —CF₂H, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃—C₁₋₆alkyl-OH, —[C₁₋₆alkyl]ₘ—COOH, —[C₁₋₆alkyl]ₘ—COOC₁₋₆alkyl, —CO—[C₁₋₆alkyl]-COOR, —C(OH)₂(C₁₋₆alkyl-OH), —C(OH)(C₁₋₆alkyl-OH)₂, —C(C₁₋₆alkyl-OH)₃, —NH₂, —NO₂, —SO₂NH₂, —P(O)R₂, —SO₂R, pyridyl (preferably ortho-pyridyl), cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH.

Particularly preferably, R¹ is CO₂H, CO₂R, CH₂OH, CH₂CH₂OH, wherein R is C₁-C₆ alkyl and each m is 0 or 1.

It has been found that polar R¹ groups result in increased potency in terms of CSF-1R inhibition. This is likely caused by favourable polar interactions at the surface of the binding pocket. The R¹ group typically protrudes out of the ligand-protein complex when the structure is bound to CSF-1R. The R¹ group(s) can thus be relatively large, and can potentially be used to increase the solubility and tune other pharmacokinetic properties of the compound, without affecting its ability to bind to the receptor. Lack of water solubility is typically a problem for pharmaceuticals and whilst our compounds are not typically water soluble in non-salt form, the increased polarity for certain R¹ groups can increase bioavailability.

When R¹ is —C₁₋₆-alkyl, R¹ may typically be ⁱPr. Typically, R¹ is —C₁₋₆-alkyl when A is pyrazolyl.

When R1 is —C(OH)₂(C₁₋₆alkyl-OH), —C(OH)(C₁₋₆alkyl-OH)₂, or —C(C₁₋₆alkyl-OH)₃, then R1 is typically —C(OH)(CH₂—OH)₂.

When R¹ is —P(O)R₂, then R1 is typically —P(O)(CH₃)₂.

When R¹ is a C₁-C₁₀ aliphatic hydrocarbyl group comprising at least one heteroatom selected from O or N, the O or N atoms are typically within the C₁-C₁₀ group, i.e. embedded within the chain, or at a terminal position, i.e. the end of the chain. Typically, said C₁-C₁₀ aliphatic hydrocarbyl group containing at least one heteroatom selected from 0 and N is a C₁-C₁₀, preferably C₁-C₈, preferably C₁-C₆ aliphatic hydrocarbyl group containing at least one group selected from ether, carboxylic acid, ester, alcohol, primary amine, secondary amine, tertiary amine, amide, preferably selected from ether, alcohol, primary amine, secondary amine, tertiary amine. The R¹ group in this case may contain a cyclic group or may be acyclic. The aliphatic hydrocarbyl group is typically, however, non-cyclic in this instance. Suitable groups therefore include C1-C10 aliphatic hydrocarbyl groups comprising at least one ether group, at least one —NH— group, at least one —NR— group, at least one —NHR group, or at least one —NR₂ group, wherein R is a C₁-C₆ alkyl group. A suitable R¹ group in this case could be, for example:

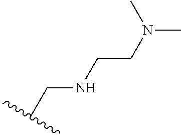 [SMILES: *CNCCN(C)C]

When R¹ is a —O—C₁-C₁₀ aliphatic hydrocarbyl group containing at least one heteroatom selected from O or N, the same considerations apply for the substitutions with the O or N groups. The group may be acyclic or may be cyclic or comprise a cyclic moiety. If it is cyclic or comprises a cyclic moiety, the R¹ group preferably is or comprises a O- or N-heterocycle, preferably an O-heterocycle. Suitable R¹ groups in this case include, for example, —O—[CH₂—CH₂—O]₂—CH₃ (i.e. ‘PEG’-type groups), with z being 1-6, preferably 1-4, more preferably 1-3, e.g. 1 or 3. Suitable R¹ groups in this case could be:

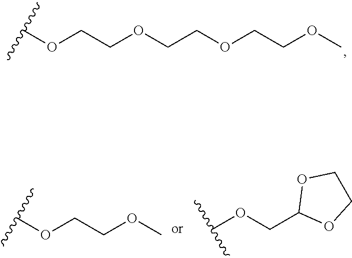

When R¹ is a —[CH₂]ₘ-heterocycle wherein said heterocycle is optionally substituted with ═O or —OH, the heterocycle may be any non-aromatic or aromatic heterocycle (typically 4-, 5- or 6-membered), e.g. containing one, two, three or four atoms selected from N, O or S (preferably O, N, or both N and O). The ═O substitution, if present, is typically on the ring (e.g. as in lactones). Particular examples of heterocycles in this instance include tetrahydropyranyl, morpholinyl, oxetanyl and tetrazolyl. Particular examples of such R¹ groups include:

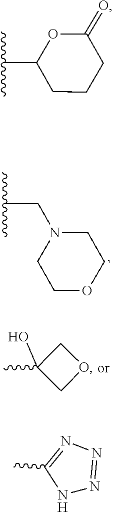

For R¹, a particularly preferred cyclopropyl or cyclobutyl substituted with an —OH group is:

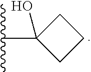 [SMILES: *C1(O)CCC1]

In a particular embodiment, R¹ is does not comprise N-methyl-piperazinyl, in particular when A is phenyl.

As mentioned above the A group may be a 6-membered hydrocarbyl or heterocyclic ring optionally substituted with at least one group R¹ group. Said hydrocarbyl ring may be selected from phenyl, cyclopentyl, cyclohexenyl, and cyclohexyl. Said heterocyclic ring is typically either a non-aromatic heterocyclic ring comprising at least one (e.g. 1 or 2) heteroatom selected from N, O, or S, e.g. piperidine, tetrahydropyran, tetrahydrofuran, dihydropyran, morpholine, or a heteroaryl ring comprising at least one (e.g. 1 or 2) heteroatom selected from N, O, or S, e.g. pyridine, thiophene, furan. In a particular embodiment, the 5- or 6-membered hydrocarbyl or heterocyclic ring in the A group is selected from phenyl, cyclohexenyl, pyridyl, morpholinyl, dihydropyranyl. Typically, the ring is a phenyl ring. Any of the above may obviously optionally be substituted with at least one R¹ group.

The A heterocyclic ring may include one, two or three heteroatoms (e.g. N, O and/or or S). A may typically be phenyl, cyclohexenyl, pyridyl, pyrazolyl, morpholinyl, 3,6-dihydro-2H-pyranyl, all optionally substituted with at least one group R¹ group. Phenyl is particularly preferred. This is in particular the case when R² and R⁴ are linked so as to form a 5- or 6-membered ring.

When A is a 5-membered heterocyclic ring, then the heterocyclic ring typically has one or two heteroatoms selected from N and/or O (as only heteroatoms). A may be pyrazolyl. Typically, when A is a 5-membered heterocyclic ring, then A is not a sulfur-containing heteroycle (such as thiophene). When A is a 5-membered heterocyclic ring and R² and R⁴ are linked so as to form a 5- or 6-membered ring, then the heterocyclic ring A typically has one, two, three or four heteroatoms selected from N and/or O. Typically, when A is a 5-membered heterocyclic ring and R² and R⁴ are linked so as to form a 5- or 6-membered ring, then A is not a sulfur-containing heterocycle (such as thiophene). Typically, when A is a 5-membered heterocyclic ring and R² and R⁴ are linked so as to form a piperidine ring, then A is not a sulfur-containing heterocycle (such as thiophene).

R² is methyl or ethyl, or deuterated or partially deuterated methyl or ethyl; or R² and R⁴ are linked so as to form a 5- or 6-membered ring. If R² and R⁴ form a ring, said ring is heterocyclic (i.e. an N-containing heterocycle with optionally further heteroatoms). Preferably said ring does not contain any substitution other than optional substitution at the R³ position. If R² and R⁴ form a ring, it is typically a 5-membered ring, i.e. such that the compound of the invention is

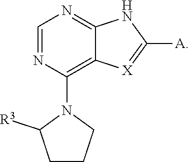

R² and R⁴ together can form a —CH₂—CH₂—CH₂— unit to form a 5-membered ring.

R² and R⁴ forming a ring may also be such that R² and R⁴ form a morpholinyl group, e.g. such that the compound of the invention is of the following formula:

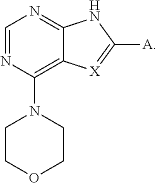

Preferably, in this case, if X is —CH, then A is not phenyl solely substituted with meta-NH₂. Alternatively viewed, the compound of the invention may be of formula (I), with the proviso that when R² and R⁴ together form a morpholinyl group, R³ is H, and X is CH, then A is not phenyl substituted with meta-NH₂. Alternatively viewed, the compound of the invention may be of formula (I), with the proviso that when R² and R⁴ together form a morpholinyl group, R³ is H, X is CH, and A is phenyl, then R¹ is not meta-NH₂. If R² and R⁴ together form a morpholinyl group, then R¹ is preferably —CH₂— morpholinyl). If R² and R⁴ together form a morpholinyl group, then A is preferably para-substituted).

In a particular embodiment, R² and R⁴ together do not form N-methyl-piperazinyl. In a particular embodiment, R² and R⁴ together do not form N-methyl-piperazinyl when A is phenyl and R¹ comprises N-methyl-piperazinyl.

Preferably, however, R² is methyl or ethyl, most preferably methyl. When R² is too large, (e.g. iso-propyl), potency to CSF-1R decreases dramatically.

If R² and R⁴ form a ring (e.g. as shown in the above structures), R³ can also be a small substituent, e.g. H or C₁-C₄ alkyl, preferably H or Me.

When R² and R⁴ are linked so as to form a 5- or 6-membered ring (preferably when X═N), then A is typically phenyl which may be optionally substituted with at least one R¹ group.

### Provisos:

If X is N and R² and R⁴ are linked so as to form a 5- or 6-membered ring, then R₃ is typically not H.

If X is N and R² and R⁴ together form a morpholinyl or pyrrolidinyl ring, then A is typically not pyridyl (e.g. not 3- or 4-pyridyl).

If X is N and R² and R⁴ together form a piperazinyl ring, then A is typically not morpholinyl.

If X is N and R² and R⁴ together form a piperazinyl ring, then A is typically not pyridyl.

When X═CH and R² and R⁴ form a 5- or 6-membered ring, then R¹ is typically not —NH₂ (particularly para-NH₂), or A is typically not phenyl with para-NH₂. When X═CH and R² and R⁴ form a 6-membered ring, or when R² and R⁴ form a 6-membered ring, then R¹ is typically not —NH₂ (particularly para-NH₂), or A is typically not phenyl with para-NH₂.

When R² and R⁴ form a 5- or 6-membered ring, then typically A is not phenyl and R₁ is not —NH₂.

When R² and R⁴ together form a morpholinyl group and R³ is H, then A is typically not a 6-membered ring substituted with para-NH₂ or —CH₂COOH (i.e. R1 is not para-NH₂ or —CH₂COOH)

When X═CH and R² and R⁴ together form a 6-membered ring, R³ is H and X is CH, then A is typically not a phenyl substituted with para-Br (i.e. A being phenyl and R1 being para-Br).

When X═CH and R₂ and R₄ form piperazinyl, then A is typically not unsubstituted phenyl, and/or R² and R⁴ do not form piperazinyl.

When X═CH and R² and R⁴ together form an imidazole ring, then A is typically not phenyl substituted with para-NH₂.

When X═CH and R² and R⁴ together form a 5-membered ring, then A is typically not a phenyl substituted with meta-NO₂.

When X═CH and R² and R⁴ are linked so as to form a 5- or 6-membered ring, then R² and R⁴ typically do not form a piperidinyl ring. Particularly, when X═CH and R² and R⁴ are linked so as to form a 5- or 6-membered ring and A is unsubstituted phenyl, then R² and R⁴ typically do not form a piperidinyl ring,

Any number of the above provisos can be combined with one another, where technically viable.

When X is N and R² and R⁴ are linked so as to form a 5- or 6-membered ring, then the compound of formula (I) is typically selected from TIA02-176 and TIA04-065.

When X is CH and R² and R⁴ are linked so as to form a 5- or 6-membered ring, then the compound of formula (I) is typically selected from TIA05-178, MS1-60-134, MS-46-93, SH-01-112, SH-01-118, SH-01-99, LR-2-067, LR-2-052.

When X is CH, R² and R⁴ are linked so as to form a 5- or 6-membered ring and A is phenyl which may be optionally substituted with at least one R¹ group, then
each R¹ is independently selected from Cl, F, I, hydroxyl, —OCF₃, —CF₃, —CF₂H, —OCF₂H, CH(CF₃)OH, —C₁-C₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, NHR, —NR₂, —SO₂NH₂, —P(O)R₂, —SO₂R, pyridyl (preferably ortho-pyridyl), cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH.

The simple act of substituting H for a small alkyl group at the R² position dramatically changes the biochemical properties of the compound. As we previously indicated, compounds with a similar skeleton were disclosed as being EGFR-TK inhibitors in WO2015/000959. The compounds reported therein all had a secondary amine group (NH), i.e. ‘R²’ was H. The present inventors have surprisingly found that substitution at the R² position (e.g. methylation, ethylation etc.) can convert the compounds from EGFR inhibitors to CSF-1R inhibitors. This, unexpectedly, opens up a whole new field of therapeutic targeting for a number of diseases. It has been found that substitution at the C-4 nitrogen blocks the EGFR activity and increases selectivity towards CSF-1R.

R³ is a 5- or 6-membered hydrocarbyl or heterocyclic ring either of which may be optionally substituted with one or more (e.g. one to three, preferably one or two) groups independently selected from C₁-C₄ alkyl (preferably C₁-C₃ alkyl, more preferably C₁-C₂ alkyl), hydroxyl, halogen, —CF₃, —CF₂H, —NR₂, —NHCOR, —CO₂H, —CO₂R, or —OR; or when R² and R⁴ are linked so as to form a 5- or 6-membered ring, R³ can also be H or a C₁-C₄ alkyl, preferably H or C₁-C₃ alkyl, preferably H or C₁-C₂ alkyl, preferably H or Me. Each R is again C₁-C₆ alkyl, preferably C₁-C₄ alkyl, C₁-C₃ alkyl or C₁-C₂ alkyl.

For R³, the 5- or 6-membered hydrocarbyl ring is typically an aliphatic or aromatic hydrocarbyl ring preferably selected from phenyl, cyclopentyl, and cyclohexyl. The 5- or 6-membered heterocyclic ring is typically either a non-aromatic heterocyclic ring comprising one heteroatom selected from N, O, or S, e.g. piperidine, tetrahydropyran, tetrahydrofuran, or a heteroaryl ring comprising one heteroatom selected from N, O, or S, e.g. pyridine, thiophene, furan (preferably pyridine or thiophene). Any of these rings can obviously be substituted, as per the optional substituents listed above. In a particular embodiment, R³ is a 5- or 6-membered hydrocarbyl or heterocyclic ring substituted with a methyl group, preferably a phenyl ring substituted with a methyl group, more preferably a phenyl ring substituted with a methyl group in the meta position.

Tetrahydropyran as R³ has a strong potency-increasing effect in addition to increasing the solubility of the compounds. In a particular embodiment, therefore, R³ is tetrahydropyranyl, preferably 4-tetrahydropyranyl, i.e.

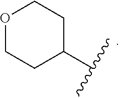 [SMILES: *C1CCOCC1]

The nature of the R³ group is important as it binds to an internal area of the CSF-1R protein. As a result, it has been found that when the R³ group is too bulky, the potency decreases. Potency is particularly increased when the R³ ring is substituted with a methyl group, e.g. a methyl group in the meta position.

When R² and R⁴ are linked so as to form a 5- or 6-membered ring, R³ can be a hydrocarbyl or heterocyclic ring as hereinbefore defined, but it can also be a small substituent, e.g. C₁-C₄ alkyl, preferably H or C₁-C₃ alkyl, preferably H or C₁-C₂ alkyl, preferably H or Me.

The R⁴ group is hydrogen, deuterium, C₁-C₄ alkyl, C₁-C₄ alkyl substituted with a hydroxyl group, or ═O (or, as described above for R², R² and R⁴ are linked so as to form a 5- or 6-membered ring). Preferably R⁴ is hydrogen, deuterium, C₁-C₂ alkyl or C₁-C₂ alkyl substituted with a hydroxyl group, or R² and R⁴ together form a —CH₂—CH₂—CH₂— unit to form a 5-membered ring, preferably R⁴ is hydrogen, methyl or —CH₂OH, most preferably R⁴ is hydrogen. Again, larger groups in this position tend to inhibit potency against CSF-1R as a result of the compound not fitting in the ‘pocket’ of the protein.

In a particular embodiment, the invention provides a compound of formula

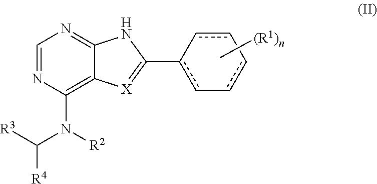

wherein:
X, R¹, R², R³, R⁴ are as hereinbefore defined;
each [symbol] independently represents a single or double bond;
n is an integer between 0 and 3; and

Preferably, n is 1 or 2. Also preferably, each [symbol] independently represents a double bond, and thus the compound is of formula (III):

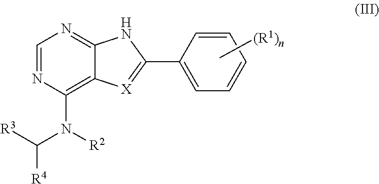

In another preferable embodiment, R² and R⁴ are such that the compound is of formula:

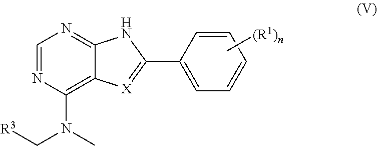

wherein R¹, R³, n, and X are as hereinbefore defined.

In an alternative embodiment, X is such that the invention provides a compound of formula

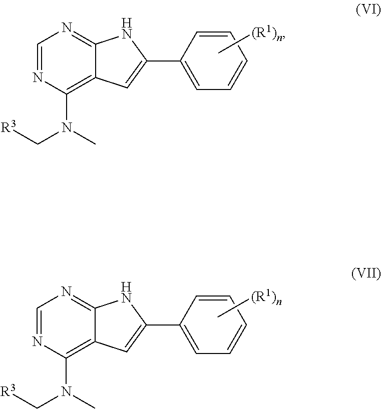

wherein R¹, R³, n, are as hereinbefore defined.

It will be appreciated that the compounds of the invention can be administered in salt, solvate, prodrug or ester form, especially salt form. The invention therefore also provides pharmaceutically acceptable salts, esters, solvates, or prodrugs of the compounds described herein, in particular pharmaceutically-acceptable salts thereof. We discuss suitable salt, solvate, prodrug or ester forms below. Typically, a pharmaceutically acceptable salt may be readily prepared by using a desired acid.

In a particular embodiment, the compounds of the invention (including compounds for use) are selected from the following. The invention provides for the grouping or exclusion of any of these compounds.

TABLE 1
Pyrrolopyrimidines (variation at C-6)

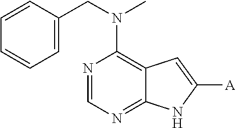

| Comp. | A-group/compound |
|---|---|
| KUL01-097 | 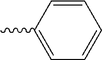 |
| TIA04-067 (comparative) | 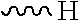 |
| KUL01-123 | 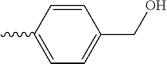 |
| TIA04-181 | 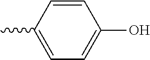 |
| KUL02-024 | 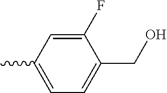 |
| KUL02-016 | 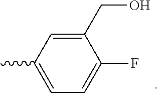 |
| TL1-62 | 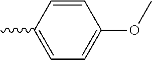 |
| TL1-86 | 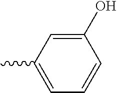 |
| TL1-78 | 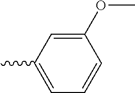 |
| TL1-122 | 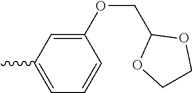 |
| TL1-70 | 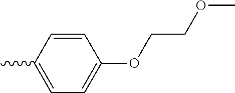 |
| TIA03-124 | 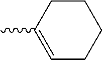 |
| NOR-4-001 | 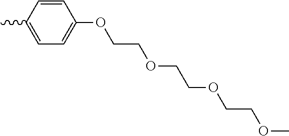 |
| KUL02-056 | 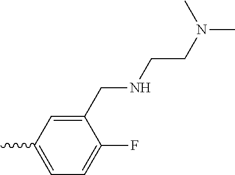 |
| KUL02-028 | 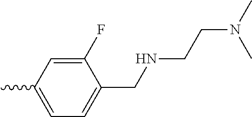 |

TABLE 2
Pyrrolopyrimidines (variation at C-6)

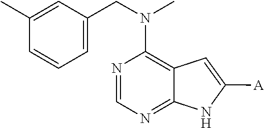

| Comp. | A-group |
|---|---|
| TIA02-056 | 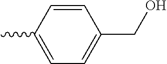 |
| FAB01-35 | 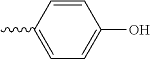 |
| FAB01-39/ FA01-72 | 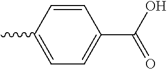 |
| TIA03-104 | 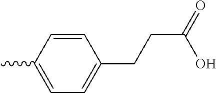 |
| FAB01-45 | 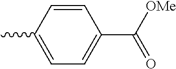 |
| FAB 01-62 | 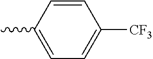 |
| FAB 01-69 | 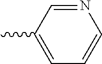 |
| FAB 01-63 | 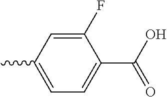 |
| FAB 01-61 | 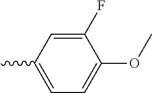 |
| FAB 01-93 | 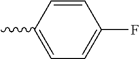 |
| FAB 01-92 | 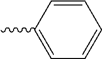 |
| LR-1-107 | 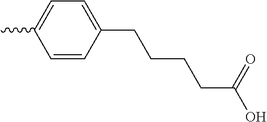 |
| LR-1-119 | 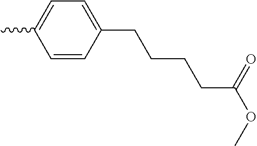 |
| LR-1-124 | 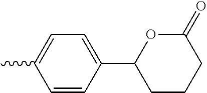 |
| LR-1-138 | 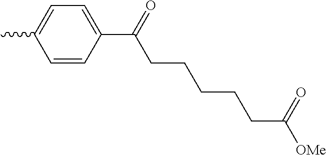 |
| NOR-4-004 | 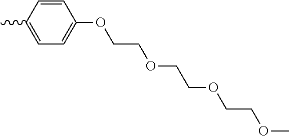 |
| FAB02-15 | 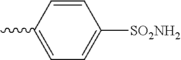 |
| FAB02-13 | 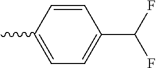 |
| FAB01-23 | 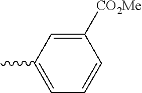 |
| FAB01-25 | 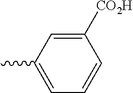 |
| LR-2-060-1 | 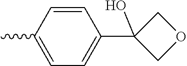 |
| LR-2-060-2 | 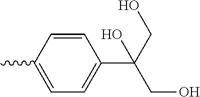 |
| LR-2-116 | 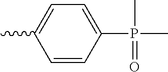 |
| LR-2-108 | 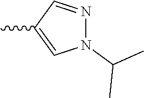 |
| FAB 02-14 | 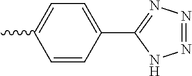 |

TABLE 3
Pyrrolopyrimidines (variation at C-6)

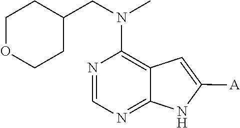

| Comp. | A-group |
|---|---|
| SH-01-18 | 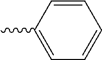 |
| TIA05-028 | 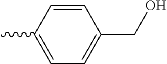 |
| TIA05-032 | 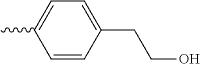 |
| SH-01-17 | 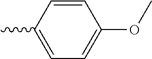 |
| SH-01-26 | 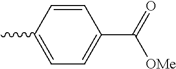 |
| TIA05-030 | 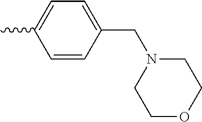 |
| SH-01-27 | 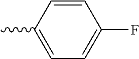 |
| SH-01-45-P1 | 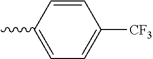 |
| SH-01-58 | 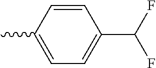 |
| SH-01-30-P1 | 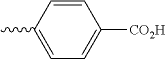 |
| SH-01-59 | 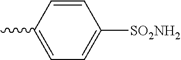 |
| SH-01-39-P1 | 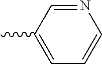 |
| SH-01-44-P1 | 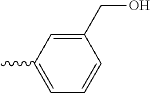 |
| SH-01-43-P1 | 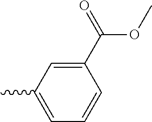 |
| SH-01-50 | 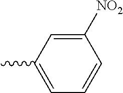 |
| SH-01-53 | 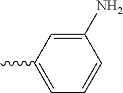 |
| FAB 02-55 | 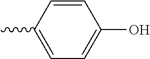 |
| FAB 02-61 | 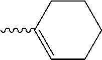 |
| SH-01-76 | 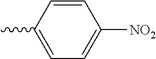 |
| SH-01-82 | 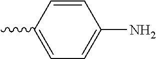 |
| SH-01-92 | 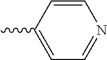 |
| TIA06-003 | 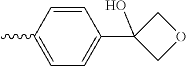 |
| FAB 04-09 | 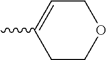 |

TABLE 4
Pyrrolopyrimidines (variation at C-4)

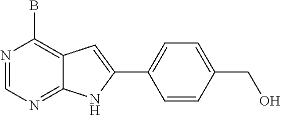

| Comp. | Amine (B-group) |
|---|---|
| TIA02-072 | 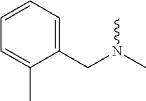 |
| TIA02-074 | 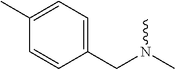 |
| TIA03-096 | 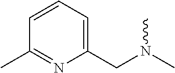 |
| TIA02-076 | 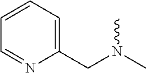 |
| TIA086 | 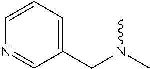 |
| TIA02-052 | 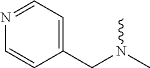 |
| TIA03-126 | 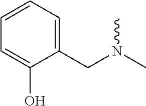 |
| TIA04-139 | 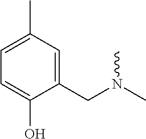 |
| TIA05-086 | 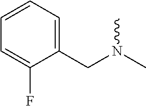 |
| KUL01-123 | 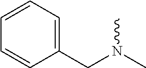 |
| TIA084 | 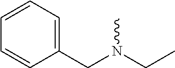 |
| TIA05-046 | 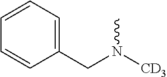 |
| TIA05-088 | 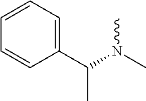 |
| TIA097 | 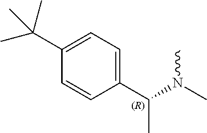 |
| TIA05-010 | 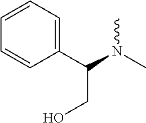 |
| TIA05-008 | 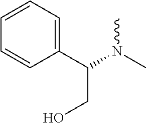 |
| TIA05-090 | 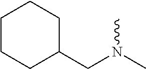 |

TABLE 5
Further suitable pyrrolopyrimidines

| Comp. | Structure |
|---|---|
| HHMT1- 170 | 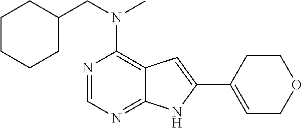 [SMILES: CN(CC1CCCCC1)c1ncnc2[nH]c(C3=CCOCC3)cc12] |
| HHMT-070 | 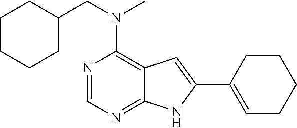 [SMILES: CN(CC1CCCCC1)c1ncnc2[nH]c(C3=CCCCC3)cc12] |
| FAB01-23 | 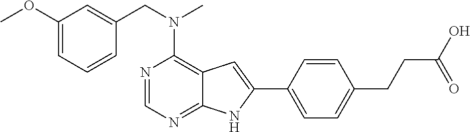 [SMILES: COc1cccc(CN(C)c2ncnc3[nH]c(-c4ccc(CCC(=O)O)cc4)cc23)c1] |
| FAB 01-24 | 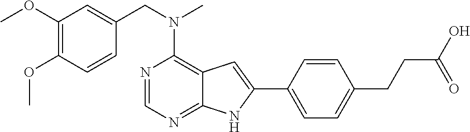 [SMILES: COc1ccc(CN(C)c2ncnc3[nH]c(-c4ccc(CCC(=O)O)cc4)cc23)cc1OC] |
| TIA05-178 | 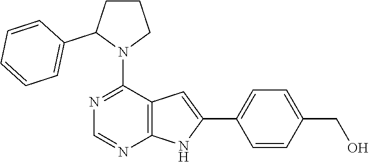 [SMILES: OCc1ccc(-c2cc3c(N4CCCC4c4ccccc4)ncnc3[nH]2)cc1] |
| MS1-60- 134 | 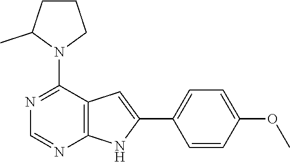 |
| MS-46-93 | 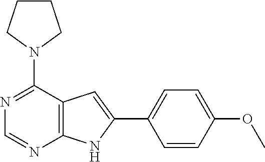 [SMILES: COc1ccc(-c2cc3c(N4CCCC4)ncnc3[nH]2)cc1] |
| KUL01-093 | 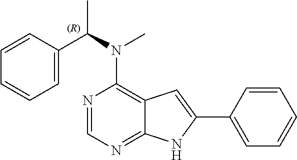 |
| SH-01-112 | 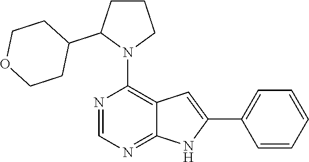 [SMILES: c1ccc(-c2cc3c(N4CCCC4C4CCOCC4)ncnc3[nH]2)cc1] |
| SH-01-118 | 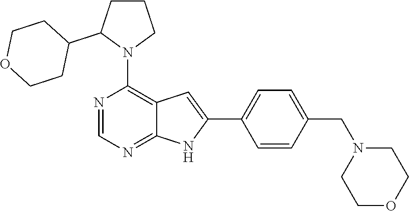 [SMILES: c1nc(N2CCCC2C2CCOCC2)c2cc(-c3ccc(CN4CCOCC4)cc3)[nH]c2n1] |
| SH-01-126 | 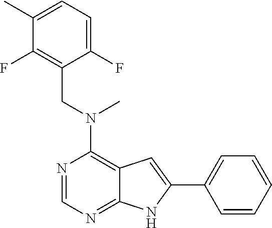 [SMILES: Cc1ccc(F)c(CN(C)c2ncnc3[nH]c(-c4ccccc4)cc23)c1F] |
| SH-01-127 | 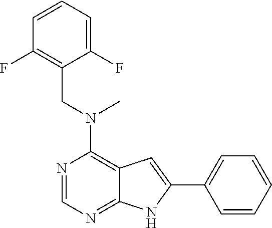 [SMILES: CN(Cc1c(F)cccc1F)c1ncnc2[nH]c(-c3ccccc3)cc12] |
| SH-01-128 | 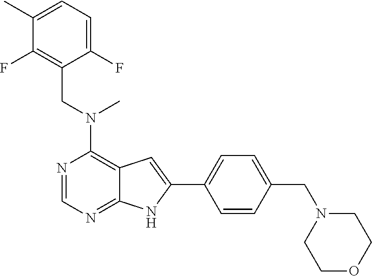 [SMILES: Cc1ccc(F)c(CN(C)c2ncnc3[nH]c(-c4ccc(CN5CCOCC5)cc4)cc23)c1F] |
| SH-01-129 | 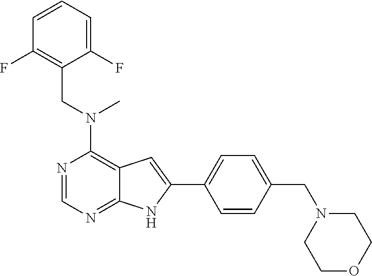 [SMILES: CN(Cc1c(F)cccc1F)c1ncnc2[nH]c(-c3ccc(CN4CCOCC4)cc3)cc12] |
| SH-01-99 | 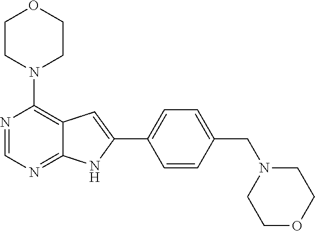 [SMILES: c1nc(N2CCOCC2)c2cc(-c3ccc(CN4CCOCC4)cc3)[nH]c2n1] |
| LR-2-067 | 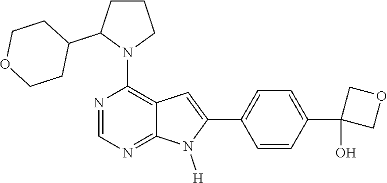 [SMILES: OC1(c2ccc(-c3cc4c(N5CCCC5C5CCOCC5)ncnc4[nH]3)cc2)COC1] |
| LR-2-052 | 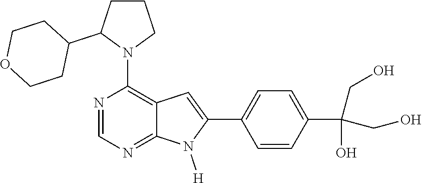 [SMILES: OCC(O)(CO)c1ccc(-c2cc3c(N4CCCC4C4CCOCC4)ncnc3[nH]2)cc1] |
| LR-2-088 (3) | 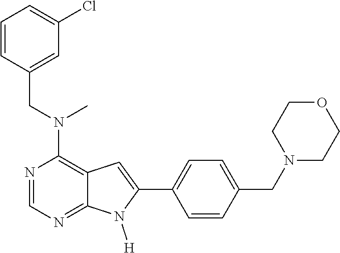 [SMILES: CN(Cc1cccc(Cl)c1)c1ncnc2[nH]c(-c3ccc(CN4CCOCC4)cc3)cc12] |
| LR-2-091 (2) | 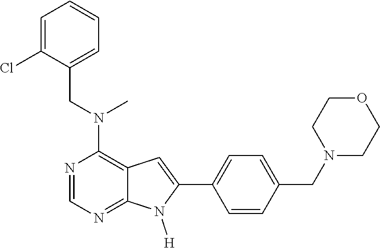 [SMILES: CN(Cc1ccccc1Cl)c1ncnc2[nH]c(-c3ccc(CN4CCOCC4)cc3)cc12] |
| LR-2-094 (2) | 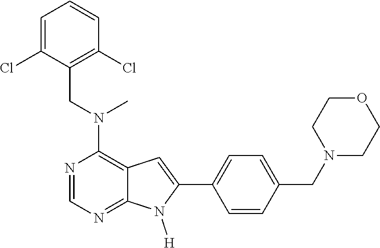 [SMILES: CN(Cc1c(Cl)cccc1Cl)c1ncnc2[nH]c(-c3ccc(CN4CCOCC4)cc3)cc12] |
| LR-2-123 | 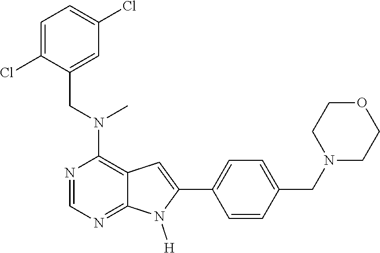 [SMILES: CN(Cc1cc(Cl)ccc1Cl)c1ncnc2[nH]c(-c3ccc(CN4CCOCC4)cc3)cc12] |
| FAB 04-15 | 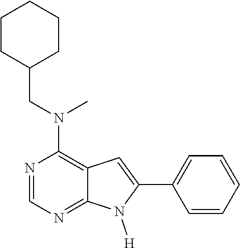 [SMILES: CN(CC1CCCCC1)c1ncnc2[nH]c(-c3ccccc3)cc12] |

TABLE 6
Purines

| Comp. | B-group (Amine) |
|---|---|
| TIA02-130 | 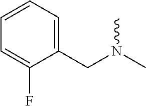 |
| TIA03-136 | 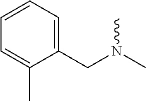 |
| TIA03-106 | 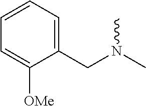 |
| TIA02-164 | 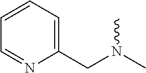 |
| TIA070 | 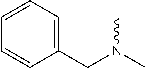 |
| EH02-21 (comparative) | 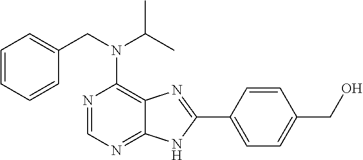 [SMILES: CC(C)N(Cc1ccccc1)c1ncnc2[nH]c(-c3ccc(CO)cc3)nc12] |
| TIA098 (comparative) | 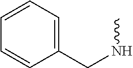 |
| TIA02-132 | 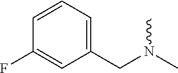 |
| TIA02-172 | 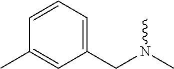 |
| TIA03-014 | 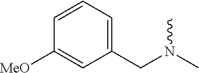 |
| TIA03-016 | 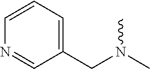 |
| TIA04-051 | 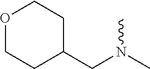 |
| TIA02-134 | 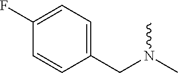 |
| TIA02-174 | 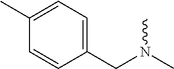 |
| TIA03-078 | 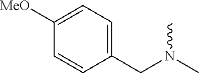 |
| TIA02-166 | 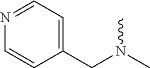 |
| TIA04-063 | 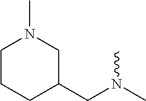 |
| TIA02-176 | 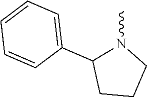 |
| TIA03-024 | 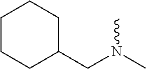 |
| TIA03-066 | 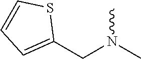 |
| TIA04-095 | 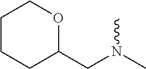 |
| TIA04-009 | 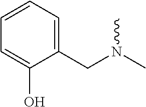 |
| TIA03-108 | 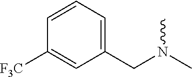 |
| TIA03-080 | 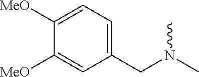 |
| TIA03-188 | 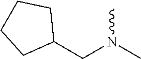 |
| TIA04-093 | 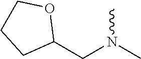 |
| TIA04-049 | 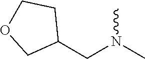 |
| TIA04-065 | 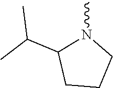 |

TABLE 7
Purines

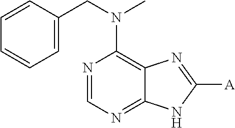

| Comp. | A-group/compounds |
|---|---|
| TIA072 | 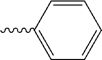 |
| TIA073 | 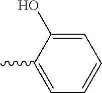 |
| JKS02-027 | 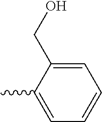 |
| TIA04-105 | 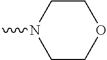 |
| JKS01-057 | 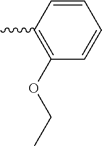 |

TABLE 8
Purines

| Comp. | Compounds |
|---|---|
| TIA04-117 | 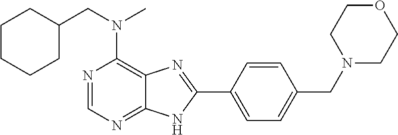 [SMILES: CN(CC1CCCCC1)c1ncnc2[nH]c(-c3ccc(CN4CCOCC4)cc3)nc12] |
| TIA03-186 | 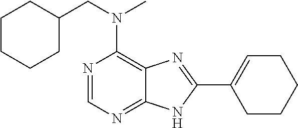 [SMILES: CN(CC1CCCCC1)c1ncnc2[nH]c(C3=CCCCC3)nc12] |
| TIA05-014 | 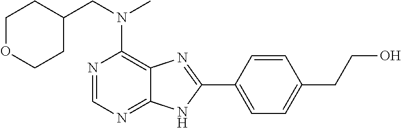 [SMILES: CN(CC1CCOCC1)c1ncnc2[nH]c(-c3ccc(CCO)cc3)nc12] |
| TIA109 | 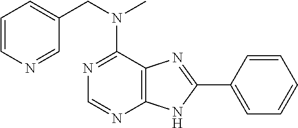 [SMILES: CN(Cc1cccnc1)c1ncnc2[nH]c(-c3ccccc3)nc12] |
| EH-93 | 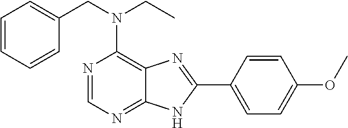 [SMILES: CCN(Cc1ccccc1)c1ncnc2[nH]c(-c3ccc(OC)cc3)nc12] |

In a particular embodiment, the compound is not one of the following compounds from Table 9.

TABLE 9

| Comp. | Structure |
|---|---|
| KUL01-093 | 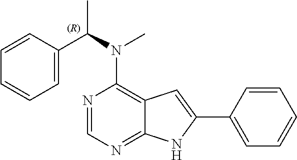 |
| TIA05-010 | 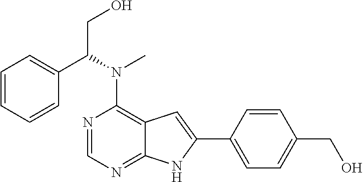 |
| TIA097 | 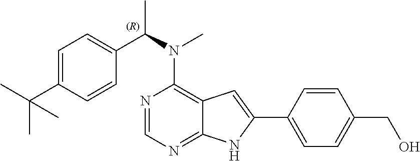 |
| TIA03-078 | 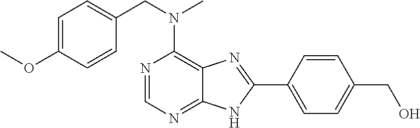 [SMILES: COc1ccc(CN(C)c2ncnc3[nH]c(-c4ccc(CO)cc4)nc23)cc1] |
| JKS01-057 | 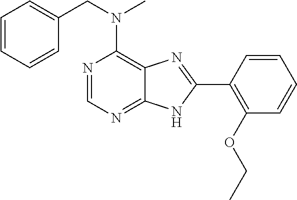 [SMILES: CCOc1ccccc1-c1nc2c(N(C)Cc3ccccc3)ncnc2[nH]1] |
| EH-93 | 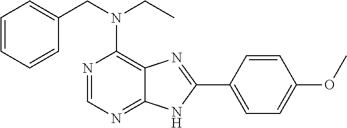 [SMILES: CCN(Cc1ccccc1)c1ncnc2[nH]c(-c3ccc(OC)cc3)nc12] |
| TIA04-063 | 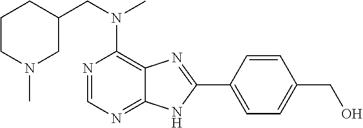 [SMILES: CN1CCCC(CN(C)c2ncnc3[nH]c(-c4ccc(CO)cc4)nc23)C1] |
| | 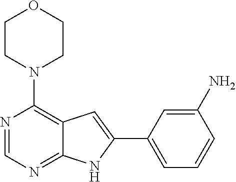 [SMILES: Nc1cccc(-c2cc3c(N4CCOCC4)ncnc3[nH]2)c1] |

One compound, more than one compound, or all of the compounds in Table 9 can be excluded. In a particular embodiment, JKS01-057 is excluded. In a further embodiment, JKS01-057 and TIA05-010 are excluded.

### Synthesis

There are provided two main routes to the compounds of the invention. In the first, a cross-coupling reaction (e.g. Suzuki-Miyaura) is used to couple the A group to the core bicyclic structure. In the second, the coupling is achieved by lithiation of the bicyclic core, followed by reaction with a ketone derivative of fragment A.

In a first aspect, therefore, there is provided a process for the formation of a compound as defined herein, said process comprising the steps of:

reacting a compound of formula:

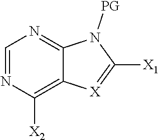

with
i) a compound of formula (RO)₂B-A or [A-BF₃] in the presence of a transition metal catalyst, and
ii) a compound of formula:

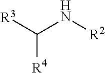

in either order (i.e. i) then ii) or ii) then i)); then
iii) removing the protecting group PG; wherein
X₁ and X₂ are halogen;
PG is a protecting group, preferably selected from -SEM, THP, BOC, Cbz, Fmoc, SO₂Ph, MOM, CO₂H, Ts;
R is OH, OMe, or (RO)₂ is pinacol (i.e. —O—C(CH₃)₂—C(CH₃)₂—O—) or MIDA ester (i.e. —O—CO—CH₂—N(CH₃)—CH₂—CO—O—),
A, X and R²-R⁴ are as defined above or in the claims.

In such a process, step i) may be carried out before step ii), or step ii) may be carried out before step i). Step iii) is always typically carried out after both steps i) and ii). Preferably the transition metal catalyst is a Pd-catalyst (e.g. PdCl₂dppf or Pd₂dba₃). Halogens X₁ and X₂ can be the same or different. Typically, they are different. In a particular embodiment, X₁ is I and X₂ is C₁. In a particular embodiment, the protecting group PG is SEM or THP. The deprotection step (removing the protecting group PG) can be achieved using any standard method, as will be known to the skilled chemist, e.g. using acid (p-TsOH, HCl, trifluoroacetic acid etc.).

MIDA esters are esters of N-methyliminodiacetic acid. SEM is —CH₂OCH₂CH₂SI(CH₃)₃. THP is tetrahydropyranyl. BOC is tert-butyloxycarbonyl. Cbz is carbobenzyloxy. Fmoc is 9-fluorenylmethyloxycarbonyl. Ts is tosyl. MOM is methoxymethyl ether. [A-BF₃]⁻ (i.e. organotrifluoroborates) may be presented as a salt with Li⁺, K⁺, or Na⁺, for example, preferably K₊.

The starting material in the above embodiment can be prepared by the following reaction:

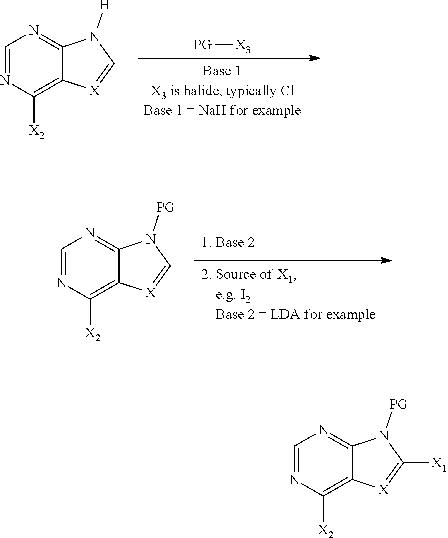

In other cases, the ‘PG’ group does not have to be presented as a reactant with a leaving group (e.g. X₃ above). For the protection of purines, for example, there is typically no leaving group. One can react with 3,4-dihydro-2H-pyran as shown below.

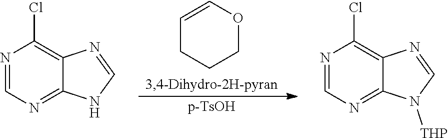

In another embodiment, the invention provides a process for the formation of a compound as defined herein, said process comprising the steps of reacting a compound of formula

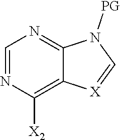

a) with a base followed by a cyclic ketone of a 6-membered hydrocarbyl or heterocyclic ring which may be optionally substituted with at least one R¹ group
b) with a compound of formula

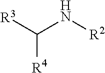

c) removing the protecting group PG
X₂ are halogen;
PG is a protecting group, preferably selected from -SEM, MOM, THP, BOC, Cbz, Fmoc, SO₂Ph, MOM, CO₂H, Ts;
A and R¹-R⁴ are as defined above or in the claims.

The cyclic ketone of a 5- or 6-membered hydrocarbyl or heterocyclic ring optionally substituted with at least one group R¹ group is a cyclic ketone of a group as defined for group A above. The cyclic ketone may be, for example, cyclohexanones, cyclopentanones, tetrahydropyranones, or piperidinones

Such a process typically leads to an unsaturated ring. If a saturated ring is required for group A, a further hydrogenation step can be used.

Typical examples of the processes of the invention are the following:

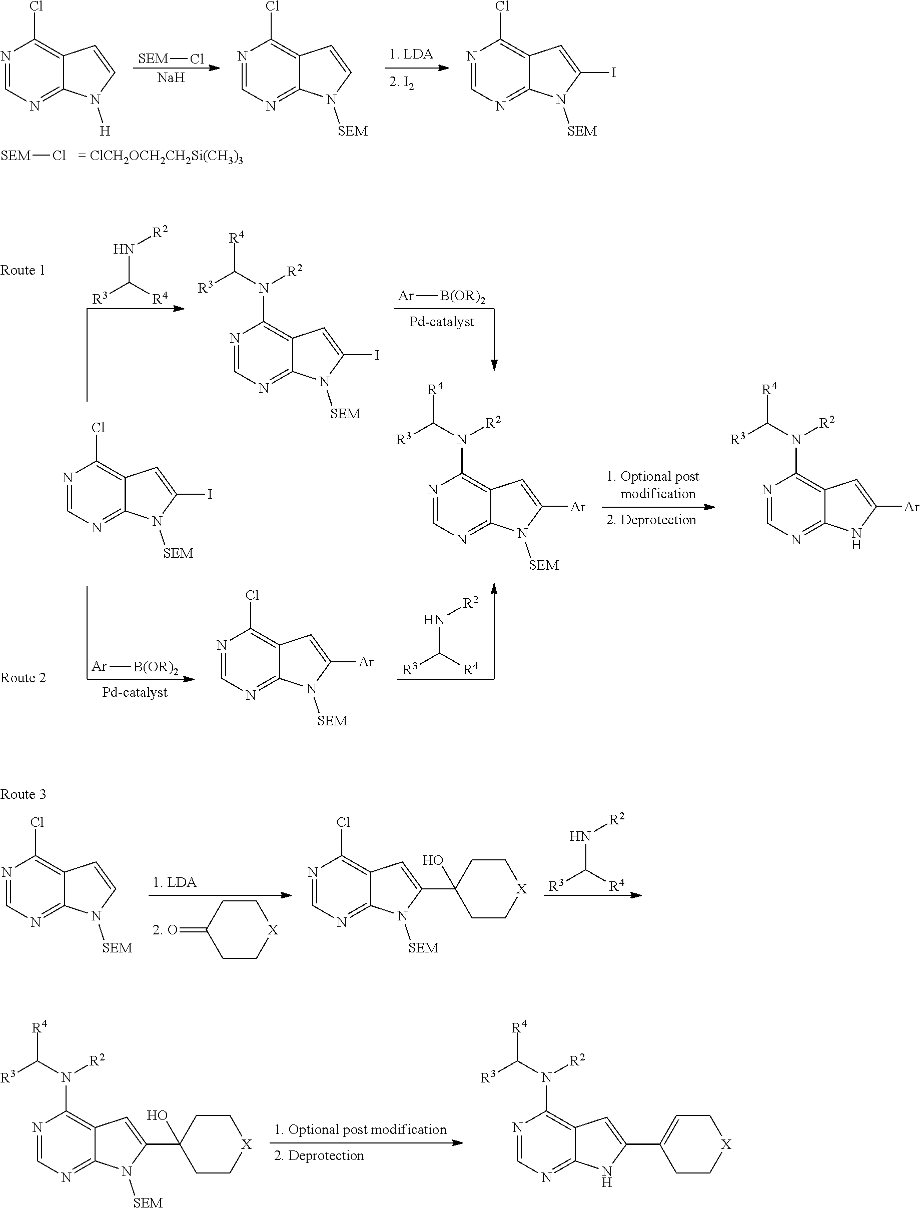

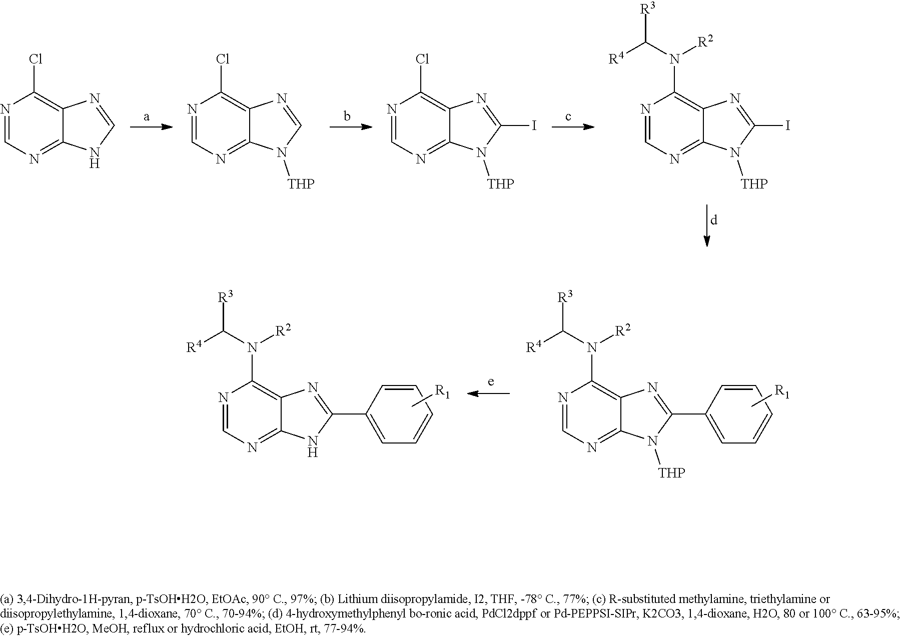

### Applications

The compounds of the present invention are potent inhibitors of CSF-1R. This results in the compounds of the invention being of particular interest in a number of therapeutic applications, given the broad role CSF-1R plays in a number of conditions.

In a particular aspect, the invention provides the compounds described herein for use in the treatment or prevention of a bone disorder, neurological disease, inflammatory disorder, cancer, or eye disease. The use may thus be therapeutic or prophylactic. Any discussion concerning the preventative/therapeutic uses or methods of the compounds of the invention apply equally to any pharmaceutical composition/formulation comprising the compounds of the invention.

The most preferred compounds of the invention typically offer CSF-1R IC₅₀ values of 50 nM or less, preferably 20 nM or less, preferably 15 nM or less, preferably 10 nM or less, such as 5 nM or less, 3 nM or less, or 1 nM or less. Some compounds even possess inhibition values of 0.5 nM or less. However, it is not a requirement for the compounds of the invention to have these IC₅₀ values. In a particular embodiment, compounds with IC₅₀ values above 10 nM, e.g. above 15 nM, e.g. above 20 nM, e.g. above 50 nM are excluded from the invention, e.g. those reported herein with IC50 values above these numbers.

In a particular embodiment, said bone disorder is osteoporosis, osteopetrosis, or osteosarcoma (for discussion, see Kodama, H. et al. Congenital osteoclast deficiency in osteopetrotic (op/op) mice is cured by injections of macrophage colony-stimulating factor. J. Exp. Med. 173, 269-272 (1991), and Smeester B A, Slipek N J, Pomeroy E J, Laoharawee K, Osum S H, Larsson A T, Williams K B, Stratton N, Yamamoto K, Peterson J J, Rathe S K, Mills L J, Hudson W A, Crosby M R, Wang M, Rahrmann E P, Moriarity B S, Largaespada D A. Bone. 2020 July; 136:115353. doi: 10.1016/j.bone.2020.115353). Preferably the bone disorder is osteoporosis.

In a particular embodiment, said neurological disease is selected from Charcot-Marie-Tooth disease, Alzheimer's disease, amyotrophic lateral sclerosis, traumatic brain injury, Hereditary diffuse leukoencephalopathy with spheroids.

In a particular embodiment, said inflammatory disorder is selected from rheumatoid arthritis and osteoarthritis.

In a particular embodiment, said cancer is selected from lung cancer, prostate cancer, colorectal cancer, stomach cancer, breast cancer, cervical cancer, multiple myeloma, ovary cancer, glioblastoma, breast cancer, malignant peripheral nerve sheath tumor, preferably multiple myeloma, ovary cancer, glioblastoma, breast cancer, malignant peripheral nerve sheath tumor.

In a particular embodiment, said eye disease is macular degeneration.

### Formulation

The compounds of the invention are preferably formulated as pharmaceutically acceptable compositions. The phrase “pharmaceutically acceptable”, as used in connection with compositions of the invention, refers to molecular entities and other ingredients of such compositions that are physiologically tolerable and do not typically produce untoward reactions when administered to a mammal (e.g. human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

The compounds of the invention can be administered in salt, solvate, prodrug or ester form, especially salt form. Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (I) and the resulting mixture evaporated to dryness (lyophilised) to obtain the acid addition salt as a solid. Alternatively, a compound of the invention may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (eg methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of Formula (I) may form solvates (e.g. hydrates) and the invention also includes all such solvates.

The term “prodrug” as used herein means a compound which is converted within the body, e.g. by hydrolysis in the blood, into its active form that has medical effects.

The compounds of the invention are proposed for use in the treatment or prevention of a bone disorder, neurological disease, inflammatory disorder, cancer, or eye disease. By treating or treatment is meant at least one of:
(i). preventing or delaying the appearance of clinical symptoms of the disease developing in a mammal;
(ii). inhibiting the disease i.e. arresting, reducing or delaying the development of the disease or a relapse thereof or at least one clinical or subclinical symptom thereof, or
(iii). relieving or attenuating one or more of the clinical or subclinical symptoms of the disease.

The benefit to a subject to be treated is either statistically significant or at least perceptible to the patient or to the physician. In general a skilled man can appreciate when “treatment” occurs.

The word prevention is used herein to cover prophylactic treatment, i.e. treating subjects who are at risk of developing a disease in question.

The compounds of the invention can be used on any animal subject, in particular a mammal and more particularly to a human or an animal serving as a model for a disease (e.g. mouse, monkey, etc.).

An “effective dose” means the amount of a compound that, when administered to an animal for treating a state, disorder or condition, is sufficient to effect such treatment. The “effective dose” will vary depending on the compound, the disease and its severity and the age, weight, physical condition and responsiveness of the subject to be treated and will be ultimately at the discretion of the attendant doctor.

While it is possible that, for use in the methods of the invention, a compound of the invention may be administered as the bulk substance, it is preferable to present the active ingredient in a pharmaceutical formulation, for example, wherein the agent is in admixture with a pharmaceutically acceptable excipient or carrier selected with regard to the intended route of administration and standard pharmaceutical practice. All of the discussion about use in the treatment or prevention of various diseases thus also applies to the formulations of the invention. In a particular embodiment, therefore, the invention provides a pharmaceutical composition comprising a compound as defined herein and at least one excipient, for use in the treatment of a bone disorder, neurological disease, inflammatory disorder, cancer, or eye disease.

The term “excipient” refers to a diluent, carrier, and/or vehicle with which an active compound is administered. The pharmaceutical compositions of the invention may contain combinations of more than one excipient or carrier. Such pharmaceutical excipients or carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the excipient any suitable binder(s), lubricant(s), suspending agent(s), coating agent(s), and/or solubilizing agent(s). Particularly preferred for the present invention are carriers suitable for immediate-release, i.e., release of most or all of the active ingredient over a short period of time, such as 60 minutes or less, and make rapid absorption of the drug possible.

It will be appreciated that pharmaceutical compositions for use in accordance with the present invention may be in the form of oral, parenteral, transdermal, inhalation, sublingual, topical, implant, nasal, or enterally administered (or other mucosally administered) suspensions, capsules or tablets, which may be formulated in conventional manner using one or more pharmaceutically acceptable carriers or excipients.

There may be different composition/formulation requirements depending on the different delivery systems. Likewise, if the composition comprises more than one active component, then those components may be administered by the same or different routes.

The pharmaceutical formulations of the present invention can be liquids that are suitable for oral, mucosal and/or parenteral administration, for example, drops, syrups, solutions, injectable solutions that are ready for use or are prepared by the dilution of a freeze-dried product but are preferably solid or semisolid as tablets, capsules, granules, powders, pellets, pessaries, suppositories, creams, salves, gels, ointments; or solutions, suspensions, emulsions, or other forms suitable for administration by the transdermal route or by inhalation.

The compounds of the invention can be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved without limitation by use of a coating resistant to conditions within the stomach but releasing the contents in the colon or other portion of the GI tract wherein a site has been identified or a delayed release can be achieved by a coating that is simply slow to disintegrate or the two (delayed and positioned release) profiles can be combined in a single formulation by choice of one or more appropriate coatings and other excipients. Such formulations constitute a further feature of the present invention.

Pharmaceutical compositions can be prepared by mixing a therapeutically effective amount of the active substance with a pharmaceutically acceptable carrier that can have different forms, depending on the way of administration. Typically composition components include one or more of binders, fillers, lubricants, odorants, dyes, sweeteners, surfactants, preservatives, stabilizers and antioxidants.

The pharmaceutical compositions of the invention may contain from 0.01 to 99% weight-per volume of the active material. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

It is within the scope of the invention for a compound as described herein to be administered in combination with another pharmaceutical, e.g. another drug with known efficacy against the disease in question. The compounds of the invention may therefore be used in combination therapy. ‘In combination’ here means in parallel; the other agents may be administered before, during, or after administration of the compound/formulation of the invention.

In particular, the compounds of the invention may be used in combination with other inhibitors with other targets, and other chemotherapeutic agents (cisplatin, taxol etc) used to treat cancerous diseases.

Also within the scope of the invention is the combination of the compounds of the invention with monoclonal antibodies.

In a particular embodiment, the compound of the invention is administered in combination with radiotherapy. ‘In combination’ here again means in parallel; the radiotherapy may be administered before, during, or after administration of the compound/formulation of the invention.

### Examples

Experimental for the Key Steps:

### General Procedure—Selective Suzuki-Miyaura Cross Coupling Reaction

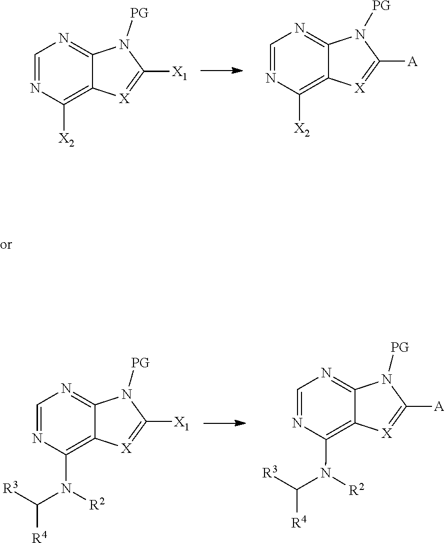

(with A, X, X¹, X², R²-R⁴, PG being as defined above, below, or in the claims) Starting material (1.0 equiv.), boronic acid (1.0-1.2 equiv.), palladium catalyst (PdCl₂dppf or Pd₂dba₃) (2-5 mol %) and potassium carbonate (3.0 equiv.) is charged in an appropriate reaction vessel. The atmosphere is evacuated and back-filled with N₂ three times before adding degassed 1,4-dioxane (6 mL/mmol starting material) and degassed water (3 mL/mmol starting material). The reaction vessel is lowered into an oil-bath set at 60-80° C. and stirred vigorously. Upon reaction completion, the reaction vessel is raised from the oil-bath and allowed to cool for 5 min. before the reaction mixture is transferred to a round-bottomed flask and the volatiles are removed by rotary evaporation. To the residue is added water (20 mL/mmol starting material), and the resulting mixture is extracted with dichloromethane (3×20 mL/mmol starting material). The combined organic layers are washed with brine (20 mL/mmol), dried with anhydrous Na₂SO₄ and filtered. The organic solvent is removed under reduced pressure and the crude product is purified by silica-gel column chromatography.

### General Procedure—Lithiation and 1,2-Nucleophilic Addition to Cyclic Ketones

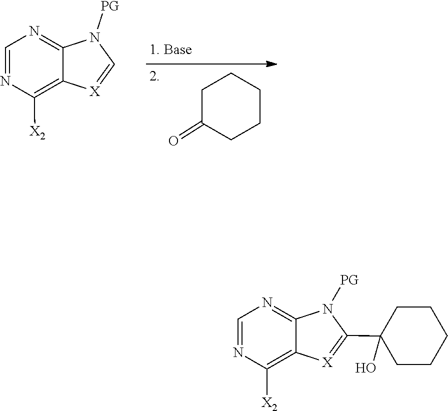

(with X, X², PG being as defined above, below, or in the claims)

4-Chloro-7-((2-(trimethylsilyl)ethoxy)methyl)-7H-pyrrolo[2,3-d]pyrimidine (1.0 equiv.) is charged in an appropriate reaction vessel, put under an atmosphere of N₂ and added tetrahydrofuran (3.5 mL/mmol starting material). After dissolution, the reaction vessel is lowered into a cooling bath of dry-ice/acetone. While stirring, lithium diisopropylamine (1.1-1.5 equiv., 2.0 M in tetrahydrofuran/heptane/ethyl benzene) is added dropwise over 30 minutes by use of a syringe pump. The mixture is then stirred for 30 minutes. The ketone (1.1-1.5 equiv.) is dissolved in tetrahydrofuran (1 mL/mmol starting material) and added dropwise by syringe pump to the reaction mixture over 30 minutes. After stirring for another 30-180 min., sat. aq. NH₄Cl (0.05 mL/mmol starting material) is added and the reaction vessel raised from the cooling bath to let warm to room temperature. The volatiles are removed under reduced pressure and the residue is added water (10 mL/mmol) and extracted with dichloromethane (3×10 mL/mmol). The combined organic phases are washed with brine (10 mL/mmol), dried using anhydrous Na₂SO₄, filtered and concentrated under reduced pressure. The crude product is purified by silica-gel flash column chromatography.

### General Procedure—Thermal Nucleophilic Amination

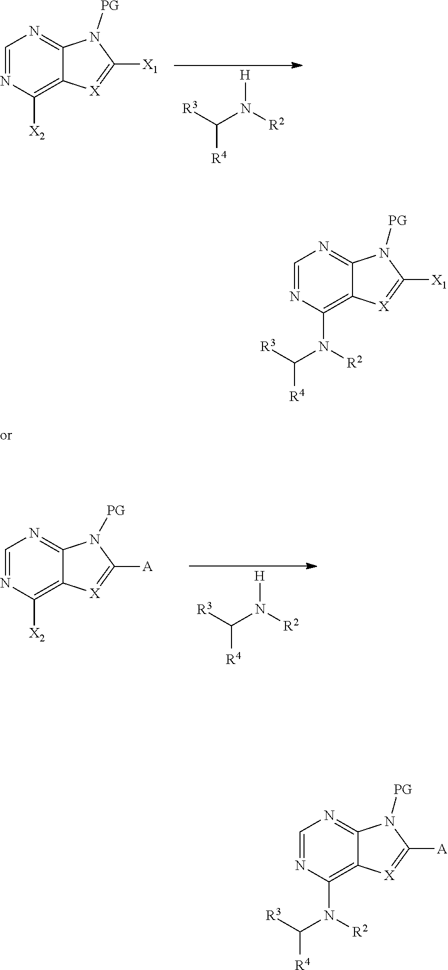

(with A, X, X¹, X², R²-R⁴, PG being as defined above, below, or in the claims)

6-Chloro-8-iodo-9-(tetrahydro-2H-pyran-2-yl)-9H-purine (1.0 equiv.), triethylamine (1.5 equiv.) and the appropriate amine nucleophile (1.5 equiv.) is dissolved in 1,4-dioxane (7.5 mL/mmol). The reaction mixture is lowered into an oil-bath set at 70° C. and stirred. Upon reaction completion, the reaction vessel is raised from the oil-bath and allowed to cool for 5 min. before the volatiles are removed under reduced pressure. The residue is added water (20 mL/mmol starting material) and extracted with ethyl acetate (3×20 mL/mmol starting material). The combined organic layers are washed with brine (20 mL/mmol), dried with anhydrous Na₂SO₄ and filtered. The organic solvent is removed under reduced pressure and the crude product is purified by silica-gel column chromatography.

Pyrrolopyrimidine Intermediates

### 4-Chloro-7-((2-(trimethylsilyl)ethoxy)methyl)-7H-pyrrolo[2,3-d]pyrimidine

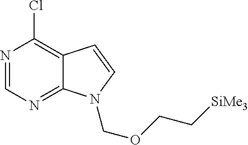 [SMILES: C[Si](C)(C)CCOCn1ccc2c(Cl)ncnc21]

The reaction was performed using a Syrris Atlas HD 2L Jacketed Reactor equipped with a Huber Ministat 125 circulator pump. Dry dimethyl formamide (200 mL) was cooled to 0° C. and added sodium hydride (3.9 g, 162.5 mmol). 4-Chloro-7H-pyrrolo[2,3-d]pyrimidine (20.1 g, 131.0 mmol) was dissolved in dry dimethyl formamide (150 mL) and added stepwise in 20 mL increments to the chilled suspension over 15 min. The reaction mixture reached an internal temperature of 6.5° C. and was left stirring to re-cool to 0° C. SEM-CI (23 mL, 132.8 mmol) was added 35 min. after the first addition of starting material. The reaction mixture reached an internal temperature of 13° C. The mixture was left stirring for 1 h, while cooling, before quenching with sat. aq. NH₄Cl (10 mL). The mixture was transferred to a round-bottom flask and concentrated in vacuo reducing the reaction volume by 345 mL. The concentrated reaction mixture was partitioned between CH₂Cl₂ (150 mL) and water (200 mL). The layers were separated and the water-phase extracted with CH₂Cl₂ (3×50 mL). The combined organic layers were washed with water (2×100 mL) and brine (225 mL), dried with anhydrous Na₂SO₄, filtered and concentrated in vacuo. The residue was purified by column chromatography on silica-gel (n-pentane/CH₂Cl₂/EtOAc: 10/10/1) yielding 32.9 g (115.8 mmol, 88%) of an off-white solid. ¹H NMR (400 MHz, DMSO-d₆): 8.68 (s, 1H), 7.87 (d, J=3.6 Hz, 2H), 6.71 (d, J=3.6 Hz, 1H), 5.65 (s, 1H), 3.52 (t, J=8.0 Hz, 2H), 0.82 (t, J=8.0 Hz, 2H), −0.10 (s, 9H).

### 4-Chloro-6-iodo-7-((2-(trimethylsilyl)-ethoxy)methyl)-7H-pyrrolo[2,3-d]pyrimidine

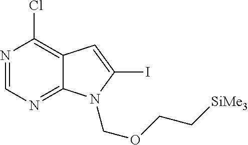 [SMILES: C[Si](C)(C)CCOCn1c(I)cc2c(Cl)ncnc21]

Under an N₂ atmosphere 4-chloro-7-((2-(trimethylsilyl)-ethoxy)methyl)-7H-pyrrolo[2,3-d]pyrimidine (5.00 g, 17.6 mmol) was dissolved in dry THF (70 mL) and cooled down to −78° C. Then, LDA (2 M in THF/n-hexane/ethylbenzene, 13.3 mL, 26.6 mmol) was added dropwise over 30 min. This was followed by drop wise addition of 12 (5.08 g, 20.1 mmol) dissolved in THF (12 mL). After another 30 min, the reaction mixture was quenched with saturated NH₄Cl solution (0.5 mL) and stirred until ambient was reached. The mixture was concentrated and diluted with 10% Na₂S₂O₃ solution (20 mL), CH₂Cl₂ (25 mL) and water (30 mL). After phase separation, the water phase was extracted with more CH₂Cl₂ (4×20 mL). The combined organic phase was dried over Na₂SO₄ and the solvent was removed under reduced pressure. The crude product was purified by silica-gel flash chromatography (n-pentane/EtOAc: 9/1, R_{f}=0.44) giving 6.60 g (16.1 mmol, 92%) of 4-chloro-6-iodo-7-((2-(trimethylsilyl)-ethoxy)methyl)-7H-pyrrolo[2,3-d]pyrimidine as a grey powder, mp. 99-101° C.; ¹H NMR (600 MHz, DMSO-d₆) δ: 8.62 (s, 1H), 7.11 (s, 1H), 5.61 (s, 2H), 3.53, (t, J=7.9 Hz, 2H), 0.82 (t, J=7.9 Hz, 2H), −1.11 (s, 9H); ¹³C NMR (150 MHz, DMSO-d₆) δ: 152.5, 150.8, 149.0, 118.6, 109.8, 91.5, 73.5, 66.0, 17.1, −1.4 (3C).

### General Procedure Chemoselective Suzuki Cross-Coupling

4-Chloro-6-iodo-7-((2-(trimethylsilyl)-ethoxy)methyl)-7H-pyrrolo[2,3-d]pyrimidine (1.0 equiv.), boronic acid (1.0-1.2 equiv.), palladium catalyst (PdCl₂dppf or Pd₂dba₃) (2-5 mol %) and potassium carbonate (3.0 equiv.) is charged in an appropriate reaction vessel. The atmosphere is evacuated and back-filled with N₂ three times before adding degassed 1,4-dioxane (6 mL/mmol starting material) and degassed water (3 mL/mmol starting material). The reaction vessel is lowered into an oil-bath set at 60-80° C. and stirred vigorously. Upon reaction completion, the reaction vessel is raised from the oil-bath and allowed to cool for 5 min. before the reaction mixture is transferred to a round-bottomed flask and the volatiles are removed by rotary evaporation. The residue is added water (20 mL/mmol starting material) and extracted with CH₂Cl₂ (3×20 mL/mmol starting material). The combined organic layers are washed with brine (20 mL/mmol), dried with anhydrous Na₂SO₄ and filtered. The organic solvent is removed under reduced pressure and the crude product is purified by silica-gel column chromatography.

### (4-(4-Chloro-7-((2-(trimethylsilyl)ethoxy)methyl)-7H-pyrrolo[2,3-d]pyrimidin-6-yl)phenyl)methanol

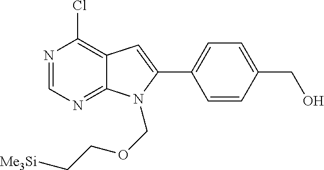 [SMILES: C[Si](C)(C)CCOCn1c(-c2ccc(CO)cc2)cc2c(Cl)ncnc21]

Following the general procedure and purification by silica-gel column chromatography (n-pentane/EtOAc 5/2, R_{f}=0.25) gave 1.13 g (2.89 mmol, 84%) as a pale yellow solid; mp. 84-85 C°; ¹H NMR (400 MHz, CDCl₃) δ: 8.67 (s, 1H), 7.79 (d, J=8.3, 2H), 7.52 (d, J=8.3 Hz, 2H), 6.71 (s, 1H), 5.62 (s, 2H), 4.80 (d, J=5.9 Hz, 2H), 3.76-3.72 (m, 2H), 1.84-1.82 (m, 1H), 0.99-0.95 (m, 2H), −0.02 (s, 9H); ¹³C NMR (100 MHz, CDCl₃) δ: 153.5, 151.5, 150.9, 143.4, 142.2, 129.8 (2C), 129.7, 127.3 (2C), 117.6, 99.5, 71.1, 64.9, 18.0, −1.4 (3C); HRMS (ASAP+, m/z): found 390.1405, calcd for C₁₉H₂₅N₃O₂SiCl, [M+H]+, 390.1405.

### 4-Chloro-6-(4-methoxyphenyl)-7-((2-(trimethylsilyl)ethoxy)methyl)-7H-pyrrolo[2,3-d]pyrimidine

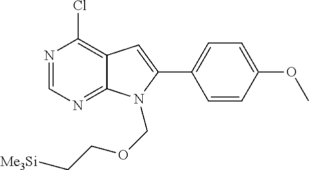 [SMILES: COc1ccc(-c2cc3c(Cl)ncnc3n2COCC[Si](C)(C)C)cc1]

Following the general procedure and purification by silica-gel column chromatography (n-pentane/EtOAc 9/1, R_{f}=0.28) gave 414 mg (1.06 mmol, 87%) as an oil; ¹H NMR (400 MHz, DMSO-d₆) δ: 8.69 (s, 1H), 7.78-7.76 (m, 2H), 7.12-7.10 (m, 2H), 6.80 (s, 1H), 5.61 (s, 2H), 3.84 (s, 3H), 3.63-3.59 (m, 2H), 0.87-0.83 (m, 2H), −0.10 (s, 9H); ¹³C NMR (100 MHz, DMSO-d₆) δ: 160.2, 153.0, 150.5, 149.9, 143.5, 130.6 (2C), 122.3, 116.9, 114.4 (2C), 98.1, 70.9, 66.2, 55.4, 17.3, −1.5 (3C); HRMS (ASAP+, m/z): found 390.1402, calcd for C₁₉H₂₅N₃O₂SiCl, [M+H]⁺, 390.1405.

### General Procedure for Lithiation and 1,2-Nucleophilic Addition to Cyclic Ketones

4-Chloro-7-((2-(trimethylsilyl)ethoxy)methyl)-7H-pyrrolo[2,3-d]pyrimidine (1.0 equiv.) is charged in an appropriate reaction vessel, put under an atmosphere of N₂ and added tetrahydrofuran (3.5 mL/mmol starting material). After dissolution, the reaction vessel is lowered into a cooling bath of dry-ice/acetone. While stirring, lithium diisopropylamine (1.1-1.5 equiv., 2.0 M in tetrahydrofuran/heptane/ethyl benzene) is added dropwise over 30 minutes by use of a syringe pump. The mixture is then stirred for 30 minutes. The ketone (1.1-1.5 equiv.) is dissolved in tetrahydrofuran (1 mL/mmol starting material) and added dropwise by syringe pump to the reaction mixture over 30 minutes. After stirring for another 30-180 min., sat. aq. NH₄Cl (0.05 mL/mmol starting material) is added and the reaction vessel raised from the cooling bath to let warm to room temperature. The volatiles are removed under reduced pressure and the residue is added water (10 mL/mmol) and extracted with CH₂Cl₂ (3×10 mL/mmol). The combined organic phases are washed with brine (10 mL/mmol), dried using anhydrous Na₂SO₄, filtered and concentrated under reduced pressure. The crude product is purified by silica-gel flash column chromatography.

### Examples

### 1-(4-Chloro-7-((2-(trimethylsilyl)ethoxy)methyl)-7H-pyrrolo[2,3-d]pyrimidin-6-yl)cyclohexan-1-ol

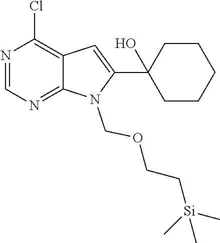 [SMILES: C[Si](C)(C)CCOCn1c(C2(O)CCCCC2)cc2c(Cl)ncnc21]

Following the general procedure using cyclohexanone and purification by silica-gel column chromatography (EtOAc:n-pentane, 1:7, R_{f}=0.21) gave 206 mg (0.539 mmol, 76%) of a pale yellow solid, mp. 72-74° C.; ¹H NMR (400 MHz, DMSO-d₆) δ:8.64 (s, 1H), 6.54 (s, 1H), 5.99 (s, 2H), 5.28 (s, 1H), 3.64 (t, 2H, J=8.0 Hz), 2.16-2.13 (m, 2H), 1.86-1.70 (m, 4H), 1.63-1.60 (m, 1H), 1.53-1.50 (m, 2H), 1.28-1.25 (m, 1H), 0.83 (t, 2H, J=8.0 Hz), −0.09 (s, 9H); 13C-NMR (100 MHz, DMSO-d₆) δ: 153.2, 150.7, 150.5, 150.0, 115.8, 96.1, 71.6, 69.5, 66.1, 36.9 (2C), 25.1, 21.3 (2C), 17.4, −1.4 (3C); HRMS (ASAP+, m/z): found 382.1713, calcd for C₁₈H₂₉N₃O₂SiCl, [M+H]⁺, 382.1718.

### 4-(4-Chloro-7-((2-(trimethylsilyl)ethoxy)methyl)-7H-pyrrolo[2,3-d]pyrimidin-6-yl)tetrahydro-2H-pyran-4-ol

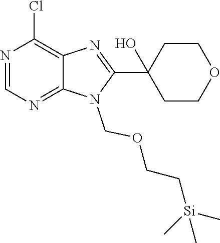 [SMILES: C[Si](C)(C)CCOCn1c(C2(O)CCOCC2)nc2c(Cl)ncnc21]

Following the general procedure using tetrahydro-4H-pyran-4-one and purification by silica-gel column chromatography (EtOAc:n-pentane, gradient from 1:4, R_{f}=0.09 to 1:2, R_{f}=0.24) gave 77 mg (0.981 mmol, 70%) of an oil; ¹H NMR (400 MHz, CDCl₃) δ: 8.64 (s, 1H), 6.55 (s, 1H), 5.98 (s, 2H), 4.00 (td, 2H, J=11.6 Hz, 2.0 Hz), 3.91-3.88 (m, 2H), 3.66 (s, 1H), 3.59 (dd, 2H, J=8.4 Hz, 8.4 Hz), 2.24 (td, 2H, J=13.2 Hz, 4.8 Hz), 2.09-2.05 (m, 2H), 0.94 (dd, 2H, J=8.8 Hz, 8.8 Hz), −0.05 (s, 9H); ¹³C NMR (100 MHz, CDCl₃) δ: 153.6, 152.2, 151.3, 147.7, 116.7, 98.4, 71.8, 68.2, 67.1, 63.3 (2C), 38.1 (2C), 18.0, −1.4 (3C); HRMS (ASAP+, m/z): found 384.1505, calcd for C₁₇H₂₇N₃O₃ClSi, [M+H]⁺, 384.1510.

### General Procedure Amination of Protected Pyrrolopyrimidines

4-Chloro-6-iodo-7-((2-(trimethylsilyl)-ethoxy)methyl)-7H-pyrrolo[2,3-d]pyrimidine (1.00 g, 1 equiv.) was dissolved in dry n-BuOH or dioxane (10 mL), added the benzyl amine (1.5-3 equiv.) and optionally N,N-diisopropylethylamine (3 equiv.). The reaction is stirred at 100-140 0C for 4-24 h. Following evaporation of solvent, the residue is added water (20 mL) and EtOAc (50 mL). After phase separation the water phase is extracted with more EtOAc (3×50 mL). The combined organic phase is then dried over MgSO₄ and concentrated at low pressure. The product were purified by silica gel column chromatography.

### Examples

### N-Benzyl-6-iodo-N-methyl-7-((2-(trimethylsilyl)ethoxy)methyl)-7H-pyrrolo[2,3-d]pyrimidin-4-amine

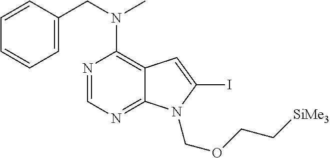 [SMILES: CN(Cc1ccccc1)c1ncnc2c1cc(I)n2COCC[Si](C)(C)C]

Following the general procedure and purification by silica-gel chromatography (n-pentane/EtOAc, 3/1, R_{f}=0.40) 3.17 g (6.42 mmol, 90%) of a solid was obtained; mp. 67-69° C.; ¹H NMR (400 MHz, DMSO-d₆) δ: 8.14 (s, 1H), 7.34-7.28 (m, 2H), 7.27-7.20 (m, 3H), 6.94 (s, 1H), 5.50 (s, 2H), 4.99 (s, 2H), 3.55-3.49 (m, 2H), 3.29 (s, 3H), 0.85-0.79 (m, 2H), −0.09 (s, 9H); ¹³C NMR (100 MHz, DMSO-d₆) δ: 155.4, 152.8, 151.3, 138.1, 128.5 (2C), 127.0, 126.9 (2C), 112.3, 104.1, 80.4, 72.7, 65.5, 52.7, 37.4, 17.1, −1.3 (3C); HRMS (ASAP+, m/z): found 495.1080, calcd for C₂₀H₂₈N₄OSiI, [M+H]⁺, 495.1077

### 6-Iodo-N-methyl-N-(3-methylbenzyl)-7-((2-(trimethylsilyl)ethoxy)methyl)-7H-pyrrolo[2,3-d]pyrimidin-4-amine

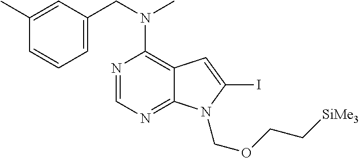 [SMILES: Cc1cccc(CN(C)c2ncnc3c2cc(I)n3COCC[Si](C)(C)C)c1]

Following the general procedure described and silica-gel column chromatography (n-pentane/EtOAc, 4:1, R_{f}=0.35) gave 1.12 g (2.20 mmol, 90%) as an oil; ¹HNMR (400 MHz, CDCl₃) δ: 8.29 (s, 1H), 7.24-7.20 (t, J=7.3 Hz, 1H), 7.10-04 (m, 3H), 6.77 (s, 1H), 5.61 (s, 2H), 4.97 (s, 2H), 3.63-3.56 (m, 2H), 3.30 (s, 3H), 2.33 (s, 3H), 0.97-0.90 (m, 2H), −0.04 (s, 9H); ¹³C NMR (100 MHz, CDCl₃) δ: 156.3, 153.5, 152.0, 138.6, 137.6, 128.8, 128.3, 127.9, 124.3, 113.0, 105.1, 76.9, 73.3, 66.5, 53.8, 37.3, 21.6 18.0, −1.3 (3C); HRMS (ASAP+, m/z): found 509.1244, calcd for C₂₁H₃₀N₄OSiI, [M+H]⁺, 509.1234.

### 6-Iodo-N-methyl-N-((tetrahydro-2H-pyran-4-yl)methyl)-7-((2-(trimethylsilyl)ethoxy)methyl)-7H-pyrrolo[2,3-d]pyrimidin-4-amine

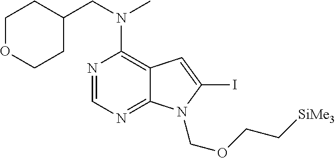 [SMILES: CN(CC1CCOCC1)c1ncnc2c1cc(I)n2COCC[Si](C)(C)C]

Following the general procedure and purification by silica-gel chromatography (gradient from n-pentane/EtOAc, 1/1 to pure EtOAc) 0.971 g, (1.93 mmol, 78%) was as an oil; ¹H NMR (600 MHz, DMSO-d₆) δ: 8.09 (s, 1H), 7.00 (s, 1H), 5.48 (s, 2H), 3.84-3.79 (m, 2H), 3.64 (d, J=7.4 Hz, 2H), 3.51 (ap. t, J=7.8 Hz, 2H), 3.32 (s, 3H) 3.27-3.18 (m, 2H), 2.06-1.96 (m, 1H), 1.52-1.46 (m, 2H), 1.30-1.21 (m, 2H), 0.81 (ap. t, J=7.8 Hz, 2H), −0.09 (s, 9H); ¹³C NMR (150 MHz, DMSO-d₆) δ: 155.3, 152.7, 151.1, 112.4, 104.2, 79.9, 72.7, 66.7 (2C), 65.5, 55.3, 38.8, 33.8, 30.2 (2C), 17.1, −1.4.

### Suzuki-Cross Coupling of Aminated Pyrrolopyrimidines

The 4-amino-6-iodo-7-((2-(trimethylsilyl)-ethoxy)methyl)-7H-pyrrolo[2,3-d]pyrimidine (1.0 equiv.), aryl boronic acid or pinacol ester (1.0-1.2 equiv.), PdCl₂dppf (2-5 mol %) and potassium carbonate (3.0 equiv.) are charged in an appropriate reaction vessel. The atmosphere is evacuated and back-filled with N₂ three times before adding degassed 1,4-dioxane (6 mL/mmol starting material) and degassed water (3 mL/mmol starting material). The reaction vessel is lowered into an oil-bath set at 60-80° C. and stirred vigorously. Upon reaction completion, the reaction vessel is raised from the oil-bath and allowed to cool for 5 min. before the reaction mixture is transferred to a round-bottomed flask and the volatiles are removed by rotary evaporation. The residue is added water (20 mL/mmol starting material) and extracted with CH₂Cl₂ (3×20 mL/mmol starting material). The combined organic layers are washed with brine (20 mL/mmol), dried with anhydrous Na₂SO₄ and filtered. The organic solvent is removed under reduced pressure and the crude product is purified by silica-gel column chromatography

### Representative Examples

### (4-(4-(Benzyl(methyl)amino)-7-((2-(trimethylsilyl)ethoxy)methyl)-7H-pyrrolo[2,3-d]pyrimidin-6-yl)phenyl)methanol

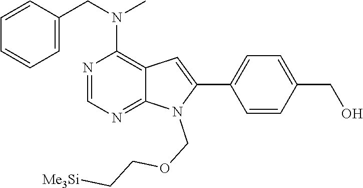 [SMILES: CN(Cc1ccccc1)c1ncnc2c1cc(-c1ccc(CO)cc1)n2COCC[Si](C)(C)C]

Following the general procedure and purification by silica-gel chromatography (n-pentane/EtOAc, 1/1, R_{f}=0.38) gave 183 mg (0.386 mmol, 73%) of an oil; ¹H NMR (600 MHz, DMSO-d₆) δ:8.24 (s, 1H), 7.67-7.66 (m, 2H), 7.41-7.39 (m, 2H), 7.34-7.31 (m, 2H), 7.27-7.23 (m, 3H), 6.78 (bs, 1H), 5.53 (s, 2H), 5.24 (t, J=5.7 Hz, 1H), 5.05 (s, 2H), 4.54 (d, J=5.7 Hz, 2H), 3.64-3.61 (m, 2H), 3.36 (s, 3H), 0.87-0.83 (m, 2H), −0.08 (s, 9H); ¹³C NMR (150 MHz, DMSO-d₆) δ:156.4, 153.0, 151.2, 142.5, 138.3, 136.5, 129.7, 128.5 (2C), 128.3 (2C), 126.9 (3C), 126.6, (2C), 102.1, 102.0, 70.3, 65.7, 62.5, 52.8, 37.4, 17.3, −1.4 (3C); HRMS (ASAP+, m/z): found 474.2448, calcd for C₂₇H₃₄N₄O₂Si, [M]⁺, 474.2451.

### (4-(4-(Methyl(3-methylbenzyl)amino)-7-((2-(trimethylsilyl)ethoxy)methyl)-7H-pyrrolo[2,3-d]pyrimidin-6-yl)phenyl)methanol

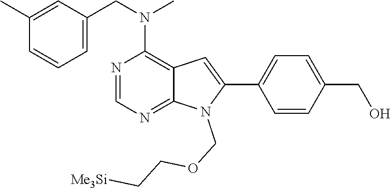 [SMILES: Cc1cccc(CN(C)c2ncnc3c2cc(-c2ccc(CO)cc2)n3COCC[Si](C)(C)C)c1]

The material was prepared using the general procedure but using Pd-PEPPSI-SIPr as catalyst. Purification by flash column chromatography (EtOAc/n-pentane: 1/2) gave 259 mg (0.529 mmol, 67%) as a clear oil; ¹H NMR (600 MHz, CDCl₃) δ: 8.39 (s, 1H), 7.69-7.64 (m, 2H), 7.45-7.40 (m, 2H), 7.24-7.18 (m, 1H), 7.11-7.06 (m, 3H), 6.55 (s, 1H), 5.56 (s, 2H), 5.03 (s, 2H), 4.74 (d, J=5.2 Hz, 2H), 3.77-3.71 (m, 2H), 3.36 (s, 3H), 2.32 (s, 3H), 1.94 (t, J=5.3 Hz, 1H), 1.00-0.93 (m, 2H), −0.02 (s, 9H); ¹³C NMR (151 MHz, CDCl₃) δ: 157.3, 153.5, 151.7, 140.9, 138.4, 137.8, 137.1, 131.2, 129.3 (2C), 128.6, 128.0, 127.8, 127.2 (2C), 124.2, 102.9, 102.0, 70.7, 66.5, 65.0, 53.8, 37.2, 21.5, 18.0, −1.4 (3C); HRMS (ASAP+, m/z): found 489.2682, calcd for C₂₃H₃₇N₄O₂Si, [M+H]⁺, 489.2686.

### Methyl 4-(4-(methyl(3-methylbenzyl)amino)-7-((2-(trimethylsilyl)ethoxy)methyl)-7H-pyrrolo[2,3-d]pyrimidin-6-yl)benzoate

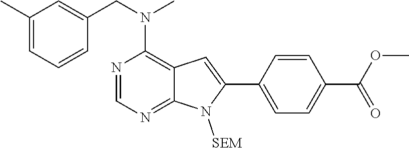

Following the general procedure and silica-gel column chromatography purification (n-pentane/EtOAc, 3:1, R_{f}=0.43) gave 946 mg (1.83 mmol, 75%). ¹H NMR (400 MHz, DMSO-d₆) δ: 8.26 (s, 1H), 8.03 (d, J=8.5 Hz, 2H), 7.90 (d, J=8.5 Hz, 2H), 7.20 (t, J=7.5 Hz, 1H), 7.02-7.09 (m, 3H), 7.01 (s, 1H), 5.59 (s, 2H), 5.03 (s, 2H), 3.88 (s, 3H), 3.67-3.57 (m, 2H), 3.37 (s, 3H), 2.26 (s, 3H), 0.89-0.81 (m, 2H), −0.09 (s, 9H); ¹³C NMR (151 MHz, DMSO-d₆) δ: 165.8, 156.6, 153.5, 151.7, 138.0, 137.6, 136.0, 134.9, 130.5, 129.4 (2C), 128.4, 128.3 (2C), 127.6 (2C), 124.0, 104.0, 102.1, 70.4, 65.7, 54.2, 52.1, 37.4, 21.0, 17.2, −1.5 (3C);

### (4-(4-(Methyl((tetrahydro-2H-pyran-4-yl)methyl)amino)-7-((2-(trimethylsilyl)ethoxy)methyl)-7H-pyrrolo[2,3-d]pyrimidin-6-yl)phenyl)methanol

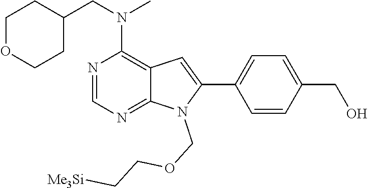 [SMILES: CN(CC1CCOCC1)c1ncnc2c1cc(-c1ccc(CO)cc1)n2COCC[Si](C)(C)C]

Following the general procedure and purification by flash column chromatography (40×150 mm silica-gel cartridge, n-pentane/i-PrOH/CH₂Cl₂: 75/20/10, 10 mL/min.) yielded 788 mg (1.63 mmol, 91%) as a clear oil. ¹H NMR (600 MHz, CDCl₃) δ: 8.33 (s, 1H), 7.72-7.68 (m, 2H), 7.47-7.43 (m, 2H), 6.60 (s, 1H), 5.54 (s, 2H), 4.75 (d, J=5.3 Hz, 2H), 3.99-3.94 (m, 2H), 3.74-3.69 (m, 4H), 3.42 (s, 3H), 3.39-3.32 (m, 2H), 2.41 (t, J=5.5 Hz, 1H), 2.18-2.08 (m, 1H), 1.65-1.60 (m, 2H), 1.47-1.37 (m, 2H), 0.98-0.92 (m, 2H), −0.04 (s, 9H); ¹³C NMR (151 MHz, CDCl₃) δ: 156.9, 153.2, 151.5, 141.0, 137.0, 131.0, 129.2 (2C), 127.2 (2C), 102.9, 102.0, 70.7, 67.7 (2C), 66.4, 64.8, 64.4, 56.6, 39.5, 34.5, 30.7 (2C), 25.3, 18.0, −1.4 (3C); HRMS (ASAP+, m/z): found 483.2787, calcd for C₂₆H₃₉N₄O₃Si, [M+H]⁺, 483.2791.

### (4-(4-(Methyl((6-methylpyridin-2-yl)methyl)amino)-7-((2-(trimethylsilyl)ethoxy)methyl)-7H-pyrrolo[2,3-d]pyrimidin-6-yl)phenyl)methanol

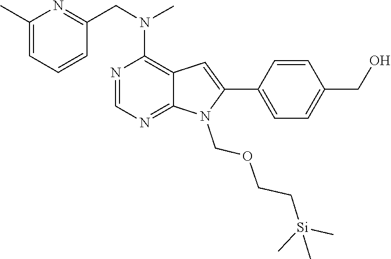 [SMILES: Cc1cccc(CN(C)c2ncnc3c2cc(-c2ccc(CO)cc2)n3COCC[Si](C)(C)C)n1]

Following the general procedure and purification by flash column chromatography (10 g silica-gel cartridge, EtOAc ln-pentane: 1/5, 10 mL/min.) yielding 90 mg (0.184 mmol, 93%) of a clear oil; ¹H NMR (400 MHz, CDCl₃) δ: 8.39 (s, 1H), 7.70-7.63 (m, 2H), 7.54-7.46 (m, 1H), 7.50-7.40 (m, 2H), 7.07-6.99 (m, 2H), 6.53 (s, 1H), 5.56 (s, 2H), 5.13 (s, 2H), 4.75 (d, J=5.1 Hz, 2H), 3.78-3.69 (m, 2H), 3.47 (s, 3H), 2.57 (s, 3H), 1.79 (t, J=5.8 Hz, 1H), 1.02-0.90 (m, 2H), −0.03 (s, 9H).

### (4-(4-(2-Phenylpyrrolidin-1-yl)-7-((2-(trimethylsilyl)ethoxy)methyl)-7H-pyrrolo[2,3-d]pyrimidin-6-yl)phenyl)methanol

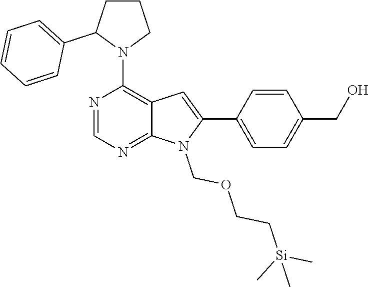 [SMILES: C[Si](C)(C)CCOCn1c(-c2ccc(CO)cc2)cc2c(N3CCCC3c3ccccc3)ncnc21]

Following the general procedure and purification by flash column chromatography (25 g silica-gel cartridge, EtOAc/n-pentane: 1/1, 10 mL/min.) yielding 83 mg (0.166 mmol, 83%) of a solid, mp. 60-100° C.; ¹H NMR (600 MHz, CDCl₃) δ: 8.34 (br s, 1H), 7.56 (br s, 2H), 7.43-7.38 (m, 2H), 7.34-7.28 (m, 2H), 7.26-7.20 (m, 3H), 6.29 (br s, 1H), 5.62 (br s, 1H), 5.55 (d, J=10.9 Hz, 1H), 5.46 (br s, 1H), 4.74 (s, 2H), 4.17 (br s, 1H), 4.04-3.95 (m, 1H), 3.71 (t, J=8.3 Hz, 2H), 2.47-2.39 (m, 1H), 2.11-1.96 (m, 3H), 1.80 (br s, 1H), 0.98-0.90 (m, 2H), −0.04 (s, 9H); ¹³C NMR (151 MHz, CDCl₃) δ: 155.1, 153.0, 152.0, 143.5, 140.6, 136.9, 131.3, 129.2 (2C), 128.6 (2C), 127.2 (2C), 126.8, 125.5 (2C), 103.3, 101.9, 70.6, 66.4, 65.0, 62.0, 49.1, 35.4, 22.1, 18.0, −1.4; HRMS (ES+, m/z): found 501.2684, calcd for C₂₉H₃₇N₄O₂Si, [M+H]⁺, 501.2686.

### N-Methyl-6-(4-(morpholinomethyl)phenyl)-N-((tetrahydro-2H-pyran-4-yl)methyl)-7-((2-(trimethylsilyl)ethoxy)methyl)-7H-pyrrolo[2,3-d]pyrimidin-4-amine

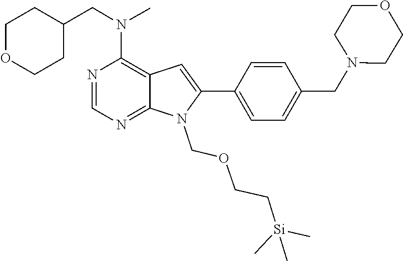 [SMILES: CN(CC1CCOCC1)c1ncnc2c1cc(-c1ccc(CN3CCOCC3)cc1)n2COCC[Si](C)(C)C]

Following the general procedure using 4-(morpholinomethyl)phenylboronic acid pinacol ester, and purification by flash column chromatography (25 g silica-gel cartridge, CH₂Cl₂/MeOH: 19/1, 10 mL/min.) yielded 97 mg (0.1760 mmol, 70%) of a clear oil; ¹H NMR (600 MHz, CDCl₃) δ: 8.34 (s, 1H), 7.69-7.65 (m, 2H), 7.44-7.40 (m, 2H), 6.60 (s, 1H), 5.57 (s, 2H), 4.01-3.95 (m, 2H), 3.76-3.69 (m, 8H), 3.56 (s, 2H), 3.43 (s, 3H), 3.40-3.33 (m, 2H), 2.51-2.47 (m, 4H), 2.19-2.10 (m, 1H), 1.67-1.61 (m, 2H), 1.48-1.38 (m, 2H), 0.98-0.92 (m, 2H), −0.04 (s, 9H); ¹³C NMR (151 MHz, CDCl₃) δ: 156.9, 153.3, 151.6, 138.0, 137.1, 130.8, 129.5 (2C), 129.0 (2C), 102.9, 101.9, 70.7, 67.7 (2C), 67.0 (2C), 66.4, 63.1, 56.6, 53.7 (2C), 39.4, 34.6, 30.8 (2C), 18.0, −1.4 (3C); HRMS (ASAP+, m/z): found 552.3369, calcd for C₃₀H₄₆N₅O₃Si, [M+H]⁺, 552.3370.

### General Procedure for SEM-Deprotection

The SEM-protected pyrrolopyrimidine (0.2 mmol, 1 equiv.) was stirred in TFA (2 mL) and CH₂Cl₂ (10 mL) at 50° C. for 3-24 hours. The reaction mixture was then concentrated in vacuo before it was taken up in MeOH (10 mL) and NH₃ (20 mL, 25% aqueous) and stirred for 2-24 h at 22° C. The reaction mixture was concentrated in vacuo, and the crude product was purified with silica-gel column chromatography. For some compounds the last step of the procedure was run with NaHCO₃ instead of ammonia and THF instead of MeOH. Yields in the range of 50-95% were seen.

### Spectroscopic Data Pyrrolopyrimidine Inhibitors

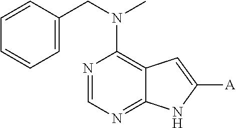

### N-Benzyl-N-methyl-6-phenyl-7H-pyrrolo[2,3-d]pyrimidin-4-amine (KUL01-097)

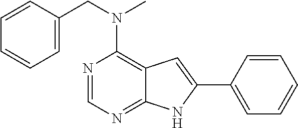 [SMILES: CN(Cc1ccccc1)c1ncnc2[nH]c(-c3ccccc3)cc12]

¹H NMR (400 MHz, DMSO-d₆) δ:12.20 (bs, 1H), 8.15 (s, 1H), 7.87-7.82 (m, 2H), 7.43-7.38 (m, 2H), 7.36-7.30 (m, 2H), 7.29-7.22 (m, 4H), 7.06 (s, 1H), 5.05 (s, 2H), 3.37 (s, 3H); ¹³C NMR (100 MHz, DMSO-d₆) δ: 156.5, 153.0, 151.2, 138.5, 133.2, 131.5, 128.8 (2C), 128.5 (2C), 127.3, 127.0 (2C), 126.9, 124.7 (2C), 103.3, 98.9, 52.7, 37.4.; HRMS (ASAP+, m/z): found 315.1605, calcd for C₂₀H₁₉N₄, [M+H]⁺, 315.1610; mp. 285-287° C. (decomp.)

### N-Benzyl-N-methyl-7H-pyrrolo[2,3-d]pyrimidin-4-amine (TIA04-067)

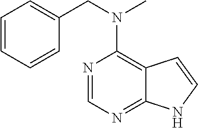 [SMILES: CN(Cc1ccccc1)c1ncnc2[nH]ccc12]

¹H NMR (600 MHz, DMSO-d₆) δ: 11.64 (s, 1H), 8.13 (s, 1H), 7.35-7.29 (m, 2H), 7.27-7.21 (m, 3H), 7.12-7.08 (m, 1H), 6.51-6.47 (m, 1H), 5.01 (s, 2H), 3.31 (s, 3H); ¹³C NMR (151 MHz, DMSO-d₆) δ: 156.8, 151.7, 150.8, 138.5, 128.5 (2C), 126.9 (2C), 126.8, 120.8, 101.8, 101.4, 52.7, 37.2; HRMS (ES+, m/z): found 239.1302, calcd for C₁₄H₁₅N₄, [M+H]⁺, 239.1297.

### (4-(4-(Benzyl(methyl)amino)-7H-pyrrolo[2,3-d]pyrimidin-6-yl)phenyl)methanol (KUL01-123)

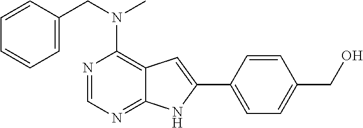 [SMILES: CN(Cc1ccccc1)c1ncnc2[nH]c(-c3ccc(CO)cc3)cc12]

¹H NMR (600 MHz, DMSO-d₆) δ: 12.15 (bs, 1H), 8.14 (s, 1H), 7.82-7.79 (m, 2H), 7.36-7.33 (m, 2H), 7.33-7.31 (m, 2H), 7.30-7.26 (m, 2H), 7.26-7.22 (m, 1H), 7.02 (s, 1H), 5.25-5.21 (m, 1H), 5.05 (s, 2H), 4.52-4.50 (m, 2H), 3.37 (s, 3H); ¹³C NMR (150 MHz, DMSO-d₆) δ: 156.4, 152.9, 151.0, 141.7, 138.5, 133.3, 129.9, 128.5 (2C), 127.0 (2C), 126.9, 126.8 (2C), 124.5 (2C), 103.3, 98.5, 62.6, 52.7, 37.4; HRMS (ASAP+, m/z): found 345.1715, calcd for C₂₁H₂₁N₄O, [M+H]⁺, 345.1715; mp. 153-157° C.

### (4-(4-(Benzylamino)-7H-pyrrolo[2,3-d]pyrimidin-6-yl)phenyl)methanol (TIA085)

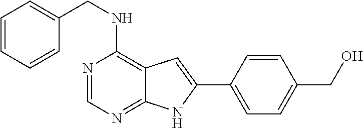 [SMILES: OCc1ccc(-c2cc3c(NCc4ccccc4)ncnc3[nH]2)cc1]

¹H NMR (600 MHz, DMSO-d₆) δ: 12.10 (s, 1H), 8.13 (s, 2H), 7.76-7.71 (m, 2H), 7.40-7.33 (m, 4H), 7.36-7.29 (m, 2H), 7.27-7.21 (m, 1H), 7.00 (s, 1H), 5.21 (s, 1H), 4.75 (d, J=6.0 Hz, 2H), 4.52 (s, 2H); ¹³C NMR (151 MHz, DMSO-d₆) δ: 155.3, 151.2 (2C), 141.8, 140.0, 133.8, 130.1, 128.3 (2C), 127.3 (2C), 127.0 (2C), 126.7, 124.4 (2C), 103.8, 95.7, 62.6, 43.3; HRMS (ASAP+, m/z): found 331.1555, calcd for C₂₀H₁₉N₄O, [M+H]⁺, 331.1559.

### 4-(4-(Benzyl(methyl)amino)-7H-pyrrolo[2,3-d]pyrimidin-6-yl)phenol (TIA04-181)

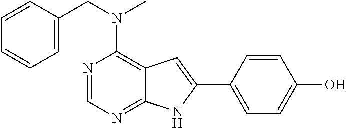 [SMILES: CN(Cc1ccccc1)c1ncnc2[nH]c(-c3ccc(O)cc3)cc12]

¹H NMR (600 MHz, DMSO-d₆) δ: 11.98 (s, 1H), 9.59 (s, 1H), 8.11 (s, 1H), 7.67-7.62 (m, 2H), 7.35-7.30 (m, 2H), 7.29-7.26 (m, 2H), 7.26-7.22 (m, 1H), 6.82 (s, 1H), 6.81-6.76 (m, 2H), 5.03 (s, 2H), 3.34 (s, 3H); ¹³C NMR (151 MHz, DMSO-d₆) δ: 157.0, 156.2, 152.7, 150.5, 138.5, 133.9, 128.5 (2C), 127.0 (2C), 126.8, 126.2 (2C), 122.6, 115.5 (2C), 103.3, 96.6, 52.7, 37.3; HRMS (ASAP+, m/z): found 331.1554, calcd for C₂₀H₁₉N₄O, [M+H]⁺ 331.1559.

### 4-(4-(Benzylamino)-7H-pyrrolo[2,3-d]pyrimidin-6-yl)phenol (JK-367)

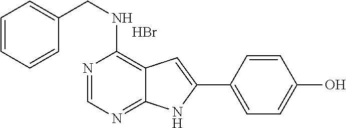 [SMILES: Oc1ccc(-c2cc3c(NCc4ccccc4)ncnc3[nH]2)cc1 ; Br]

Experimental data is given in Kaspersen et al. Eur. J. Med. Chem. (2011), 46(12), 6002-6014.

### (4-(4-(Benzyl(methyl)amino)-7H-pyrrolo[2,3-d]pyrimidin-6-yl)-2-fluorophenyl)methanol (KUL02-024)

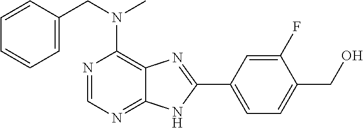 [SMILES: CN(Cc1ccccc1)c1ncnc2[nH]c(-c3ccc(CO)c(F)c3)nc12]

¹H NMR (600 MHz, DMSO-d₆) δ: 12.20 (bs, 1H), 8.16 (s, 1H), 7.70-7.66 (m, 2H), 7.49-7.45 (m, 1H), 7.35-7.30 (m, 2H), 7.29-7.22 (m, 3H), 7.17 (bs, 1H), 5.26 (t, J=5.7 Hz, 1H), 5.05 (s, 2H), 4.54 (d, J=5.7 Hz), 3.37 (s, 3H); ¹³C NMR (150 MHz, DMSO-d₆) δ: 160.0 (d, J=243.2 Hz), 156.5, 153.0, 151.4, 138.4, 132.3 (d, J=8.8 Hz), 132.0 (d, J=2.6 Hz), 129.5 (d, J=5.5 Hz), 128.5 (2C), 127.8 (d, J=15.4 Hz), 127.1 (2C), 126.9, 120.4 (d, J=2.1 Hz), 110.9 (d, J=23.8 Hz), 103.2, 99.8, 56.6, 52.6, 37.4; HRMS (ASAP+, m/z): found 363.1616, calcd for C₂₁H₂₀N₄OF, [M+H]⁺, 363.1621; mp. 265-269° C.

### (5-(4-(Benzyl(methyl)amino)-7H-pyrrolo[2,3-d]pyrimidin-6-yl)-2-fluorophenyl)methanol (KUL02-016)

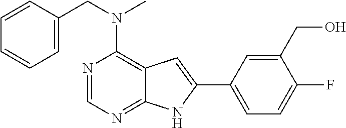 [SMILES: CN(Cc1ccccc1)c1ncnc2[nH]c(-c3ccc(F)c(CO)c3)cc12]

¹H NMR (600 MHz, DMSO-d₆) δ: 12.19 (bs, 1H), 8.15 (s, 1H), 7.96-7.92 (m, 1H), 7.78-7.74 (m, 1H), 7.35-7.30 (m, 2H), 7.29-7.26 (m, 2H), 7.26-7.23 (m, 1H), 7.23-7.18 (m, 1H), 6.99 (bs, 1H), 5.28 (t, J=5.6 Hz, 1H), 5.05 (s, 2H), 4.56 (d, J=5.6 Hz, 2H), 3.37 (s, 3H); ¹³C NMR (150 MHz, DMSO-d₆) δ: 159.1 (d, J=245.6 Hz), 156.4, 153.0, 151.1, 138.4, 132.6, 129.5 (d, J=15.5 Hz), 128.5 (2C), 127.9 (d, J=3.1 Hz), 127.0 (2C), 126.9, 125.9 (d, J=4.7 Hz), 125.1 (d, J=8.3 Hz), 115.3 (d, J=21.9 Hz), 103.2, 98.6, 56.9 (d, J=3.4 Hz), 52.6, 37.3; HRMS (ASAP+, m/z): found 363.1617, calcd for C₂₁H₂₀N₄OF, [M+H]⁺, 363.1621; mp. 212-215° C.

### N-Benzyl-6-(4-methoxyphenyl)-N-methyl-7H-pyrrolo[2,3-d]pyrimidin-4-amine (TL1-62)

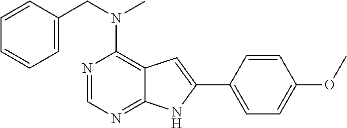 [SMILES: COc1ccc(-c2cc3c(N(C)Cc4ccccc4)ncnc3[nH]2)cc1]

¹H NMR (600 MHz, DMSO-d₆) δ: 12.06 (s, 1H), 8.13 (s, 1H), 7.77 (d, J=8.9 Hz, 2H), 7.34-7.31 (m, 2H), 7.28-7.23 (m, 3H). 6.97 (d, J=8.9 Hz, 2H), 6.90 (s, 1H), 5.04 (s, 2H), 3.78 (s, 3H), 3.36 (s, 3H); ¹³C NMR (150 MHz, DMSO-d₆) δ: 158.7, 156.2, 152.8, 150.7, 138.5, 133.3, 128.5 (2C), 127.0 (2C), 126.8, 126.1 (2C) 124.2, 114.2 (2C), 103.3, 97.3, 55.2, 52.7, 37.3; HRMS (ASAP+, m/z): found 345.1715, calcd for C₂₁H₂₁N₄O, [M+H]⁺, 345.1715; mp. 250-252° C.

### N-Benzyl-6-(4-methoxyphenyl)-7H-pyrrolo[2,3-d]pyrimidin-4-amine (JK-356)

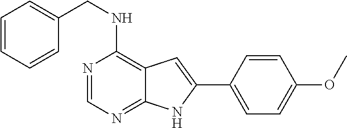 [SMILES: COc1ccc(-c2cc3c(NCc4ccccc4)ncnc3[nH]2)cc1]

Experimental data is given in Kaspersen et al. Eur. J. Med. Chem. (2011), 46(12), 6002-6014.

### 3-(4-(Benzyl(methyl)amino)-7H-pyrrolo[2,3-d]pyrimidin-6-yl)phenol (TL1-86)

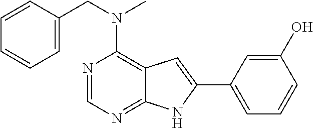 [SMILES: CN(Cc1ccccc1)c1ncnc2[nH]c(-c3cccc(O)c3)cc12]

¹H NMR (600 MHz, DMSO-d₆) δ: 12.09 (s, 1H), 9.46 (s, 1H), 8.14 (s, 1H), 7.34-7.32 (m, 2H), 7.28-7.23 (m, 4H), 7.20-7.18 (m, 2H), 6.92 (s, 1H), 6.71-6.69 (m, 1H), 5.04 (s, 2H), 3.36 (s, 3H); ¹³C NMR (150 MHz, DMSO-d₆) δ: 157.6, 156.4, 152.8, 151.1, 138.4, 133.5, 132.8, 129.7, 128.4 (2C), 127.0 (2C), 126.9, 115.7, 114.4, 111.7, 103.1, 98.6, 52.7, 37.3; HRMS (ASAP+, m/z): found 331.1558, calcd for C₂₀H₁₉N₄O, [M+H]⁺, 331.1559; mp. 252-254° C.

### N-Benzyl-6-(3-methoxyphenyl)-N-methyl-7H-pyrrolo[2,3-d]pyrimidin-4-amine (TL1-78)

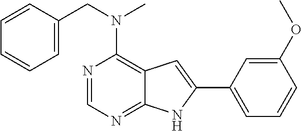 [SMILES: COc1cccc(-c2cc3c(N(C)Cc4ccccc4)ncnc3[nH]2)c1]

¹H NMR (600 MHz, DMSO-d₆) δ: 12.17 (s, 1H), 8.15 (s, 1H), 7.43-7.41 (m, 2H), 7.35-7.24 (m, 6H), 7.07 (s, 1H), 6.85-6.83 (m, 1H), 5.05 (s, 2H), 3.81 (s, 3H), 3.37 (s, 3H); ¹³C NMR (150 MHz, DMSO-d₆) δ: 159.7, 156.5, 152.9, 151.2, 138.4, 133.1, 132.8, 129.8, 128.5 (2C), 127.0 (2C), 126.9, 117.1, 113.1, 110.0, 103.2, 99.2, 55.2, 52.7, 37.4; HRMS (ASAP+, m/z): found 345.1712, calcd for C₂₁H₂₁N₄O, [M+H]⁺, 345.1715; mp. 210-212° C.

### 6-(3-((1,3-Dioxolan-2-yl)methoxy)phenyl)-N-benzyl-N-methyl-7H-pyrrolo[2,3-d]pyrimidin-4-amine (TL1-122)

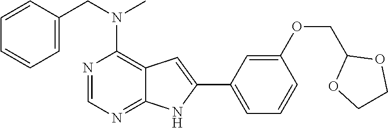 [SMILES: CN(Cc1ccccc1)c1ncnc2[nH]c(-c3cccc(OCC4OCCO4)c3)cc12]

¹H NMR (600 MHz, DMSO-d₆) δ: 12.15 (s, 1H), 8.15 (s, 1H), 7.47-7.43 (m, 2H), 7.34-7.23 (m, 6H), 7.10 (s, 1H), 6.87-6.84 (m, 1H) 5.23 (t, J=4.1, 1H), 5.05 (s, 2H), 4.06 (d, J=4.1 Hz, 2H), 3.99-3.86 (m, 4H), 3.37 (s, 3H); ¹³C NMR (150 MHz, DMSO-d₆) δ: 158.6, 156.5, 152.9, 151.3, 138.4, 133.0, 132.9, 129.9, 128.5 (2C), 127.0 (2C), 126.9, 117.4, 113.7, 110.4, 103.2, 101.3, 99.3, 68.2, 64.5 (2C), 52.6, 37.4; HRMS (ASAP+, m/z): found 417.1927, calcd for C₂₄H₂₅N₄O₃, [M+H]⁺, 417.1927; mp. 203-205° C.

### N-Benzyl-6-(4-(2-methoxyethoxy)phenyl)-N-methyl-7H-pyrrolo[2,3-d]pyrimidin-4-amine (TL1-70)

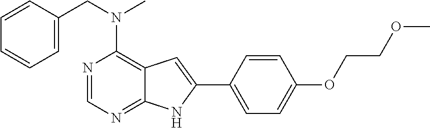 [SMILES: COCCOc1ccc(-c2cc3c(N(C)Cc4ccccc4)ncnc3[nH]2)cc1]

¹H NMR (600 MHz, DMSO-d₆) δ: 12.06 (s, 1H), 8.13 (s, 1H), 7.76 (d, J=8.9 Hz, H), 7.34-7.31 (m, 2H), 7.28-7.23 (m, 3H), 6.99 (d, J=8.9 Hz, 2H), 6.90 (s, 1H), 5.04 (s, 2H), 4.14-4.10 (m, 2H) 3.70-3.64 (m, 2H) 3.35 (s, 3H), 3.31 (s, 3H); ¹³C NMR (150 MHz, DMSO-d₆) δ: 157.9, 156.2, 152.7, 150.7, 138.5, 133.3, 128.5 (2C), 127.0 (2C), 126.8, 126.1 (2C), 124.2, 114.7 (2C), 103.3, 97.3, 70.3, 66.9, 58.1, 52.6, 37.3; HRMS (ASAP+, m/z): found 398.1974, calcd for C₂₃H₂₅N₄O₂, [M+H]⁺, 389.1978; mp. 217-219° C.

### N-Benzyl-6-(cyclohex-1-en-1-yl)-N-methyl-7H-pyrrolo[2,3-d]pyrimidin-4-amine (TIA03-124)

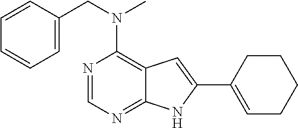 [SMILES: CN(Cc1ccccc1)c1ncnc2[nH]c(C3=CCCCC3)cc12]

¹H NMR (600 MHz, DMSO-d₆) δ: 11.69 (s, 1H), 8.10 (s, 1H), 7.35-7.29 (m, 2H), 7.27-7.21 (m, 3H), 6.43 (s, 1H), 6.38-6.34 (m, 1H), 4.99 (s, 2H), 3.30 (s, 3H), 2.32-2.26 (m, 2H), 2.19-2.13 (m, 2H), 1.70-1.61 (m, 2H), 1.63-1.55 (m, 2H); ¹³C NMR (151 MHz, DMSO-d₆) δ: 156.2, 152.6, 150.9, 138.5, 135.2, 128.5 (2C), 127.8, 126.9 (2C), 126.8, 122.8, 102.5, 97.5, 52.7, 37.3, 25.0, 24.9, 22.1, 21.8; HRMS (ES+, m/z): found 319.1925, calcd for C₂₀H₂₃N₄, [M+H]⁺, 319.1923.

### N-Benzyl-6-(4-(2-(2-(2-methoxyethoxy)ethoxy)ethoxy)phenyl)-N-methyl-7H-pyrrolo[2,3-d]pyrimidin-4-amine (NOR-4-001)

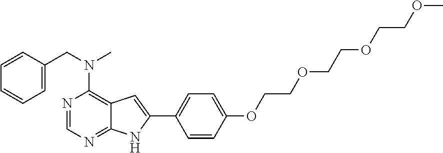 [SMILES: COCCOCCOCCOc1ccc(-c2cc3c(N(C)Cc4ccccc4)ncnc3[nH]2)cc1]

¹H NMR (400 MHz, DMSO-d₆) δ: 12.09 (s, 1H), 8.13 (s, 1H), 7.80-7.76 (m, 2H), 7.37-7.22 (m, 5H), 7.02-6.98 (m, 2H), 6.91 (s, 1H), 5.05 (s, 2H), 4.15-4.10 (m, 2H), 3.77-3.72 (m, 2H), 3.62-3.58 (m, 2H), 3.56-3.51 (m, 4H), 3.46-3.42 (m, 2H), 3.37 (s, 3H), 3.24 (s, 3H); ¹³C NMR (100 MHz, DMSO-d₆) δ: 159.1, 154.3, 148.2, 146.3, 139.0, 133.9, 129.0 (6C), 127.5, 126.6, 115.2 (2C), 111.4, 103.7, 80.2, 71.7 (2C), 70.4, 70.1, 69.4, 58.6, 55.1, 37.8; HRMS (ASCI/ASAP, m/z): found 477.2496, calcd for C₂₇H₃₂N₄O₄, [M+H]⁺, 477.2502; mp. 123-126° C.

### N¹-(5-(4-(Benzyl(methyl)amino)-7H-pyrrolo[2,3-d]pyrimidin-6-yl)-2-fluorobenzyl)-N²,N²-dimethylethane-1,2-diamine (KUL02-056)

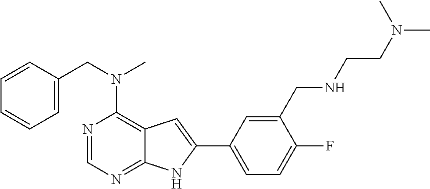 [SMILES: CN(C)CCNCc1cc(-c2cc3c(N(C)Cc4ccccc4)ncnc3[nH]2)ccc1F]

¹H NMR (600 MHz, DMSO-d₆) δ: 12.16 (bs, 1H), 8.15 (s, 1H), 7.92-7.88 (m, 1H), 7.76-7.72 (m, 1H), 7.35-7.31 (m, 2H), 7.29-7.26 (m, 2H), 7.26-7.23 (m, 1H), 7.22-7.18 (m, 1H), 7.00 (bs, 1H), 5.05 (s, 2H), 3.75 (s, 2H), 3.37 (s, 3H), 2.59 (t, J=6.4 Hz, 2H), 2.32 (t, J=6.4 Hz, 2H), 2.09 (s, 6H). ¹³C NMR (150 MHz, DMSO-d₆) δ: 159.8 (d, J=245.1 Hz), 156.4, 153.0, 151.1, 138.4, 132.6, 128.5 (2C), 128.0 (d, J=16.0 Hz), 127.8 (d, J=3.0 Hz), 127.03 (2C), 126.96 (d, J=4.7 Hz), 126.9, 124.9 (d, J=8.0 Hz), 115.4 (d, J=22.5 Hz), 103.2, 98.6, 58.7, 52.6, 46.3 (d, J=1.5 Hz), 46.2, 45.2 (2C), 37.4; HRMS (ASAP+, m/z): found: 433.2510, calcd for C₂₅H₃₀N₆F, [M+H]⁺, 433.2516; mp. 145-147° C.

### N¹-(4-(4-(Benzyl(methyl)amino)-7H-pyrrolo[2,3-d]pyrimidin-6-yl)-2-fluorobenzyl)-N²,N²-dimethylethane-1,2-diamine (KUL02-028)

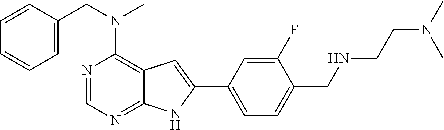 [SMILES: CN(C)CCNCc1ccc(-c2cc3c(N(C)Cc4ccccc4)ncnc3[nH]2)cc1F]

¹H NMR (600 MHz, DMSO-d₆) δ: 12.19 (bs, 1H), 8.15 (s, 1H), 7.70-7.65 (m, 2H), 7.46-7.43 (m, 1H), 7.35-7.30 (m, 2H), 7.29-7.23 (m, 3H), 7.16 (bs, 1H), 5.05 (s, 2H), 3.73 (s, 2H), 3.37 (s, 3H), 2.56 (t, J=6.4 Hz, 2H), 2.32 (t, J=6.4 Hz, 2H), 2.10 (s, 6H); ¹³C NMR (150 MHz, DMSO-d₆) δ: 160.8 (d, J=242.5 Hz), 156.5, 53.0, 151.4, 138.4, 132.1 (d, J=8.7 Hz), 132.0 (d, J=1.8 Hz), 130.7 (d, J=5.6 Hz), 128.5 (2C), 127.0 (2C), 126.9, 126.3 (d, J=15.5 Hz), 120.4 (d, J=2.4 Hz), 111.0 (d, J=24.2 Hz), 103.2, 99.7, 58.7, 52.6, 46.1, 45.8 (d, J=1.5 Hz), 45.2 (2C), 37.4; HRMS (ASAP+, m/z): found 433.2509, calcd for C₂₅H₃₀N₆F, [M+H]⁺, 433.2516; mp. 190-196° C.

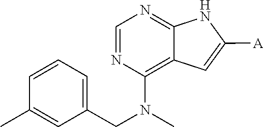

### N-Methyl-N-(3-methylbenzyl)-7H-pyrrolo[2,3-d]pyrimidin-4-amine (TIA04-069)

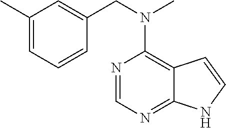 [SMILES: Cc1cccc(CN(C)c2ncnc3[nH]ccc23)c1]

¹H NMR (400 MHz, DMSO-d₆) δ: 11.64 (s, 1H), 8.12 (s, 1H), 7.24-7.16 (m, 1H), 7.13-7.08 (m, 1H), 7.08-6.99 (m, 3H), 6.48 (s, 1H), 4.96 (s, 2H), 3.29 (s, 3H), 2.26 (s, 3H); ¹³C NMR (101 MHz, DMSO-d₆) δ: 156.8, 151.7, 150.8, 138.4, 137.6, 128.4, 127.5, 127.4, 123.9, 120.8, 101.8, 101.4, 52.7, 37.2, 21.1; HRMS (ES+, m/z): found 253.1458, calcd for C₁₅H₁₇N₄, [M+H]⁺, 253.1453.

### (4-(4-(Methyl(3-methylbenzyl)amino)-7H-pyrrolo[2,3-d]pyrimidin-6-yl)phenyl)methanol (TIA02-056)

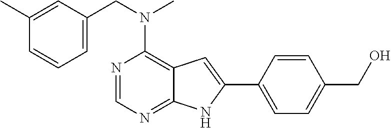 [SMILES: Cc1cccc(CN(C)c2ncnc3[nH]c(-c4ccc(CO)cc4)cc23)c1]

¹H NMR (600 MHz, DMSO-d₆) δ: 12.14 (s, 1H), 8.14 (s, 1H), 7.82-7.78 (m, 2H), 7.37-7.32 (m, 2H), 7.24-7.18 (m, 1H), 7.11-7.08 (m, 1H), 7.08-7.04 (m, 2H), 7.03 (s, 1H), 5.19 (t, J=5.7 Hz, 1H), 5.01 (s, 2H), 4.50 (d, J=5.7 Hz, 2H), 3.35 (s, 3H), 2.27 (s, 3H); ¹³C NMR (151 MHz, DMSO-d₆) δ: 156.4, 152.9, 151.1, 141.7, 138.4, 137.6, 133.3, 130.0, 128.4, 127.58, 127.55, 126.8 (2C), 124.5 (2C), 124.1, 103.2, 98.5, 62.6, 52.6, 37.3, 21.1; HRMS (ASAP+, m/z): found 359.1868, calcd for C₂₂H₂₃N₄O, [M+H]⁺, 359.1872.

### 4-(4-(Methyl(3-methylbenzyl)amino)-7H-pyrrolo[2,3-d]pyrimidin-6-yl)phenol (FAB01-35)

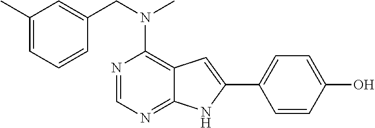 [SMILES: Cc1cccc(CN(C)c2ncnc3[nH]c(-c4ccc(O)cc4)cc23)c1]

¹H NMR (600 MHz, DMSO-d₆) δ: 11.99 (s, 1H), 9.59 (s, 1H), 8.12 (s, 1H), 7.66 (d, J=8.6 Hz, 2H), 7.22 (t, J=7.6 Hz, 1H), 7.09 (s, 1H), 7.04-7.07 (m, 2H), 6.83 (s, 1H), 6.82-6.77 (m, 2H), 5.00 (s, 2H), 3.34 (s, 3H), 2.28 (s, 3H); ¹³C NMR (151 MHz, DMSO-d₆) δ: 157.0, 156.2, 152.7, 150.6, 138.5, 137.6, 133.8, 128.4, 127.6, 127.5, 126.2 (2C), 124.1, 122.6, 115.5 (2C), 103.3, 96.6, 52.6, 37.2, 21.1; HRMS (ES+, m/z): found 345.1826, calcd. C₂₁H₂₀N₄O, [M+H]⁺, 345.163711 [M+H].

### 4-(4-(Methyl(3-methylbenzyl)amino)-7H-pyrrolo[2,3-d]pyrimidin-6-yl)benzoic acid (FAB01-39)

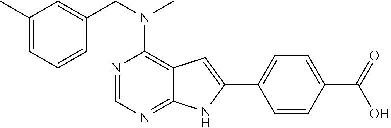 [SMILES: Cc1cccc(CN(C)c2ncnc3[nH]c(-c4ccc(C(=O)O)cc4)cc23)c1]

¹H NMR (600 MHz, DMSO-d₆) δ: 12.32 (s, 1H), 8.17 (s, 1H), 7.91-7.98 (m, J=8.4 Hz, 4H), 7.25 (s, 1H), 7.21 (t, J=7.6 Hz, 1H), 7.10 (s, 1H), 7.08-7.05 (m, 2H), 5.03 (s, 2H), 3.37 (s, 3H), 2.27 (s, 3H); ¹³C NMR (MHz, DMSO-d₆) δ: 167.0, 156.6, 153.3, 151.7, 138.2, 137.6, 135.3, 132.1, 129.7 (3C), 128.4, 127.6 (2), 124.4 (2C), 124.1, 103.3, 101.0, 52.5, 37.4, 21.0; HRMS (ES+, m/z): found 373.1668 (calcd. C₂₂H₂₀N₄O₂, 373.1586 [M+H]⁺); mp. 241-243° C.

### 3-(4-(4-(Benzyl(methyl)amino)-7H-pyrrolo[2,3-d]pyrimidin-6-yl)phenyl)propanoic acid (TIA03-104)

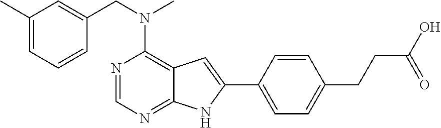 [SMILES: Cc1cccc(CN(C)c2ncnc3[nH]c(-c4ccc(CCC(=O)O)cc4)cc23)c1]

¹H NMR (400 MHz, DMSO-d₆) δ: 12.13 (s, 1H), 8.14 (s, 1H), 7.78-7.72 (m, 2H), 7.30-7.24 (m, 2H), 7.24-7.17 (m, 1H), 7.11-7.02 (m, 3H), 7.00 (s, 1H), 5.00 (s, 2H), 3.35 (s, 3H), 2.83 (t, J=7.6 Hz, 2H), 2.55 (t, J=7.6 Hz, 2H), 2.27 (s, 3H); ¹³C NMR (101 MHz, DMSO-d₆) δ: 173.8, 156.4, 152.9, 151.1, 140.1, 138.4, 137.6, 133.3, 129.4, 128.7 (2C), 128.4, 127.6 (2C), 124.7 (2C), 124.1, 103.3, 98.4, 52.6, 37.4, 35.1, 30.1, 21.1; HRMS (ES+, m/z): found 401.1979, calcd for C₂₄H₂₅N₄O₂ [M+H]⁺ 401.1978.

### Methyl 4-(4-(methyl(3-methylbenzyl)amino)-7H-pyrrolo[2,3-d]pyrimidin-6-yl)benzoate (FAB 01-45)

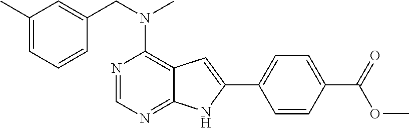

¹H NMR (600 MHz, DMSO-d₆) δ: 12.35 (s, 1H), 8.18 (s, 1H), 7.9-8.3 (m, J=8.5 Hz, J=2.3 Hz 4H), 7.28 (s, 1H), 7.22 (t, J=7.6 Hz, 1H), 7.10 (s, 1H), 7.04-7.08 (m, 2H), 5.03 (s, 2H), 3.87 (s, 3H), 3.38 (s, 3H), 2.28 (s, 3H); ¹³C NMR (151 MHz, DMSO-d₆) δ: 165.9, 156.7, 153.4, 151.9, 138.2, 137.6, 136.0, 131.9, 129.7 (2C), 128.4, 127.7, 127.6 (2C), 124.6 (2C), 124.1, 103.4, 101.4, 52.6, 52.1, 37.5, 21.1; HRMS (ES+, m/z): found 387.1826, calcd. C₂₂H₁₉F₃N₄, [M+H]⁺, 387.1742; mp. 232-233.5° C.

### N-Methyl-N-(3-methylbenzyl)-6-(4-(trifluoromethyl)phenyl)-7H-pyrrolo[2,3-d]pyrimidin-4-amine (FAB 01-62)

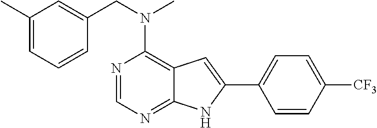 [SMILES: Cc1cccc(CN(C)c2ncnc3[nH]c(-c4ccc(C(F)(F)F)cc4)cc23)c1]

¹H NMR (600 MHz, DMSO-d₆) δ: 12.38 (s, 1H), 8.19 (s, 1H), 8.08 (d, J=8.2 Hz, 2H), 7.77 (d, J=8.3 Hz, 2H), 7.29 (s, 1H), 7.22 (t, J=7.6 Hz, 1H), 7.10 (s, 1H), 7.08-7.05 (m, 2H), 5.03 (s, 2H), 3.38 (s, 3H), 2.28 (s, 3H); ¹³C NMR (151 MHz, DMSO-d₆) δ: 156.7, 153.3, 151.8, 138.2, 137.6, 135.5, 131.5 128.4, 127.6 (2C), 127.0 (q, J_{CF}=30 Hz) 126.1 (q, J_{CF}=273 Hz), 125.6 (q, J_{CF}=3.2 Hz, 2C), 125.0 (2C), 124.1, 103.3, 101.3, 52.6, 37.4, 2.1; HRMS (ES+, m/z): found 397.1643, calcd. C₂₂H₁₉F₃N₄, [M+H]⁺, 397.1561; mp. 220-223° C.

### N-Methyl-N-(3-methylbenzyl)-6-(pyridin-3-yl)-7H-pyrrolo[2,3-d]pyrimidin-4-amine (FAB01-69)

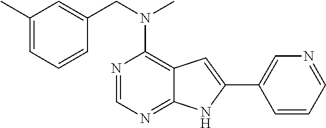 [SMILES: Cc1cccc(CN(C)c2ncnc3[nH]c(-c4cccnc4)cc23)c1]

¹H NMR (600 MHz, DMSO-d₆) δ: 12.34 (s, 1H), 9.10 (d, J=2.3 Hz, 1H), 8.47 (dd, J=4.7, 1.5 Hz, 1H), 8.20-8.22 (m, 1), 8.18 (s, 1H) 7.45 (dd, J=8.0, 4.7 Hz, 1H), 7.25 (s, 1H), 7.22 (t, J=7.6 Hz, 1H), 7.10 (s, 1H) 7.04-7.08 (m, 2H), 5.03 (s, 2H), 3.38 (s, 3H), 2.28 (s, 3H). ¹³C NMR (151 MHz, DMSO-d₆) δ: 156.6, 153.2, 151.6, 148.0, 146.1, 138.3, 137.6, 131.6, 130.1, 128.5, 127.6 (2C), 127.5, 124.2, 123.8, 103.2, 100.3, 52.6, 37.5, 21.1. HRMS (ES+, m/z): found 330.1725, calcd for C₂₀H₁₉N₅, [M+H]⁺, 330.16404; mp. 220-223° C.

### 2-Fluoro-4-(4-(methyl(3-methylbenzyl)amino)-7H-pyrrolo[2,3-d]pyrimidin-6-yl)benzoic acid (FAB 01-63)

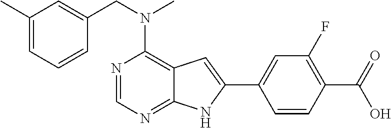 [SMILES: Cc1cccc(CN(C)c2ncnc3[nH]c(-c4ccc(C(=O)O)c(F)c4)cc23)c1]

¹H NMR (600 MHz, DMSO-d₆) δ: 12.34 (s, 1H), 8.18 (s, 1H), 7.78-7.89 (m, 3H), 7.35 (s, 1H), 7.22 (t, J=7.6 Hz, 1H), 7.10 (s, 1H), 7.06 (m, 2H), 5.03 (s, 2H), 3.37 (s, 3H), 2.28 (s, 3H); ¹³C NMR (151 MHz, DMSO-d₆) δ: 164.9, 161.5 (d, J=256 Hz) 156.7, 153.4, 152.0, 142.4, 138.2, 137.6, 136.7 132.3, 131.0, 129.2 128.4, 127.6 (2C), 124.1, 120.1 (d=3.4 Hz), 112.2 (d, J=25 Hz), 103.3, 102.0 53.0, 37.5, 21.1; HRMS (ES+, m/z): found 391.1738, calcd. C₂₂H₂₀N₄O₂, [M+H]⁺, 391.1492.

### 6-(3-Fluoro-4-methoxyphenyl)-N-methyl-N-(3-methylbenzyl)-7H-pyrrolo[2,3-d]pyrimidin-4-amine (FAB 01-61)

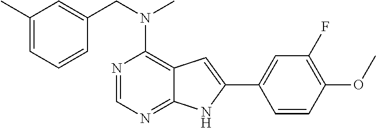

¹¹H NMR (600 MHz, DMSO-d₆) δ: 12.13 (s, 1H), 8.14 (s, 1H), 7.76 (dd, J=12.9, 2.1 Hz, 1H), 7.65 (dd, J=8.64, 1.3 Hz 1H), 7.22 (m, 2H), 7.09 (s, 1H), 7.03-7.07 (m, 3H), 5.01 (s, 2H), 3.87 (s, 3H), 3.35 (s, 3H), 2.27 (s, 3H); ¹³C NMR (151 MHz, DMSO-d₆) δ: 156.4, 152.9, 151.6 (d, J=245 Hz), 151.0, 146.3 (d, J=10.9 Hz), 138.4, 137.6, 132.1, 128.4, 127.6 (2C), 124.8 (d, J=7.6 Hz), 124.1, 121.0 (d, J=2.7 Hz), 114.2 (d, J=1.6 Hz), 112.4 (d, J=20 Hz), 103.2, 98.6, 56.1, 52.5, 37.3, 21.1; HRMS (ES+, m/z): found 377.1783, calcd for C₂₂H₂₁FN₄O, [M+H]⁺, 376.1770; mp. 236-238° C.

### 6-(4-fluorophenyl)-N-methyl-N-(3-methylbenzyl)-7H-pyrrolo[2,3-d]pyrimidin-4-amine (FAB 01-93)

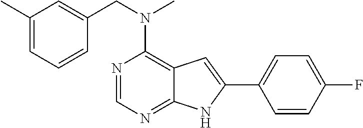 [SMILES: Cc1cccc(CN(C)c2ncnc3[nH]c(-c4ccc(F)cc4)cc23)c1]

¹H NMR (600 MHz, DMSO-d₆) δ: 12.20 (s, 1H), 8.16 (s, 1H), 7.86-7.93 (m, 2H), 7.24-7.30 (m, 2H), 7.22 (t, J=7.5 Hz, 1H), 7.09 (s, 1H), 7.04-7.09 (m, 3H), 5.02 (s, 2H), 3.36 (s, 3H), 2.27 (s, 3H); ¹³C NMR (151 MHz, DMSO d₆) δ: 161.4 (d, J=243 Hz), 156.5, 153.0, 151.2, 138.4, 137.6, 132.3, 128.4, 128.2 (d, J=3 Hz), 127.6 (2C), 126.7 (d, J=8 Hz, 2C), 124.1, 115.7 (d, J=22 Hz, 2C), 103.2, 98.9, 52.6, 37.4, 21.1.

### N-methyl-N-(3-methylbenzyl)-6-phenyl-7H-pyrrolo[2,3-d]pyrimidin-4-amine (FAB 01-92)

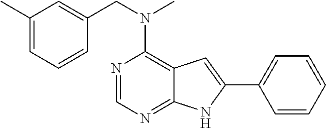 [SMILES: Cc1cccc(CN(C)c2ncnc3[nH]c(-c4ccccc4)cc23)c1]

¹H NMR (600 MHz, DMSO-d₆) δ: 12.18 (s, 1H), 8.15 (s, 1H), 7.85 (d, J=7.8 Hz, 2H), 7.42 (t, J=7.7 Hz, 2H), 7.28 (t, J=7.4 Hz, 1H), 7.22 (t, J=7.5 Hz, 1H), 7.11-7.00 (m, 4H), 5.02 (s, 2H), 2.27 (s, 3H); ¹³C NMR (151 MHz, DMSO-d₆) δ: 156.5, 153.0, 151.2, 138.4, 137.6, 133.2, 131.5, 128.8, 128.5, 127.6 (2C), 127.3, 124.7, 124.1, 103.3, 98.9, 52.6, 37.4, 21.1.

### 5-(4-(4-(Methyl(3-methylbenzyl)amino)-7H-pyrrolo[2,3-d]-pyrimidin6-yl)phenyl)pentanoic acid (LR-1-107)

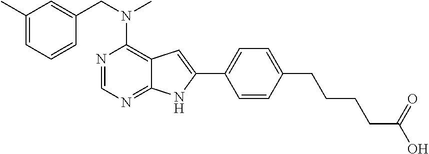 [SMILES: Cc1cccc(CN(C)c2ncnc3[nH]c(-c4ccc(CCCCC(=O)O)cc4)cc23)c1]

¹H NMR (600 MHz, DMSO-d₆) δ: 12.13 (s, 1H), 11.97 (s, 1H), 8.15 (s, 1H), 7.75-7.74 (d, J=8.1 Hz, 2H), 7.23-7.20 (m, 3H), 7.09 (s, 1H), 7.06-7.05 (m, 2H), 6.99 (s, 1H), 5.01 (s, 2H), 3.35 (s, 3H), 2.60-2.58 (t, J=7.5, 2H), 2.27 (s, 3H), 2.25-2.22 (t, J=7.3, 2H), 1.62-1.57 (m, 2H), 1.54-1.49 (m, 2H); ¹³C NMR (150 MHz, DMSO-d₆) δ: 174.4, 156.3, 152.8, 150.9, 141.3, 138.4, 137.6, 133.4, 129.0, 128.7 (2C), 128.4, 127.6 (2C), 124.7 (2C), 124.1, 103.2, 98.3, 52.6, 37.3, 34.5, 33.5, 30.2, 24.1, 21.1; HRMS (ASAP+, m/z): found 429.2285, calcd for C₂₆H₂₉N₄O₂, [M+H]⁺, 429.2291; mp. 216-219° C.

### Methyl 5-(4-(4-(methyl(3-methylbenzyl)amino)-7H-pyrrolo[2,3-d]pyrimidin-6-yl)phenyl)pentanoate (LR-1-119)

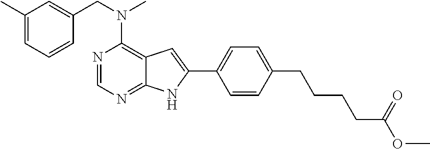

¹H NMR (400 MHz, DMSO-d₆) δ: 12.12 (s, 1H), 8.14 (s, 1H), 7.76-7.74 (d, J=8.1 Hz, 2H), 7.23-7.19 (m, 3H), 7.09-7.05 (m, 3H), 6.98 (s, 1H), 5.00 (s, 2H), 3.57 (s, 3H), 3.35 (s, 3H), 2.60-2.57 (t, J=6.9, 2H), 2.35-2.31 (t, J=7.1, 2H), 2.27 (s, 3H), 1.61-1.52 (m, 4H); ¹³C NMR (100 MHz, DMSO-d₆) δ: 173.3, 156.4, 152.9, 151.0, 141.2, 138.4, 137.6, 133.4, 129.1, 128.7 (2C), 128.4, 127.6 (2C), 124.7 (2C), 124.1, 103.2, 98.3, 52.6, 51.2, 37.3, 34.4, 33.1, 30.1, 24.0, 21.1; HRMS (ASAP+, m/z): found 443.2448, calcd for C₂₇H₃₁N₄O₂, [M+H]⁺, 443.2447; mp. 162-164° C.

### 7-(4-(4-(Methyl(3-methylbenzyl)amino)-7H-pyrrolo[2,3-d]pyrimidin-6-yl)phenyl)-7-oxoheptanoate (LR-1-138)

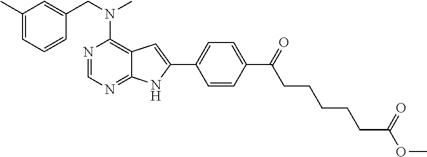

¹H NMR (600 MHz, DMSO-d₆) δ: 12.34 (s, 1H), 8.18 (s, 1H), 7.99-7.98 (br s, 4H), 7.26 (s, 1H). 7.22-7.20 (t, J=7.5 Hz, 1H), 7.09 (s, 1H), 7.07-7.05 (m, 2H), 5.03 (s, 2H), 3.58 (s, 3H), 3.38 (s, 3H), 3.03-3.00 (t, J=7.1, 2H), 2.32-2.29 (t, J=7.4, 2H), 2.27 (s, 3H), 1.64-1.54 (m, 4H), 1.36-1.31 (m, 2H); ¹³C NMR (150 MHz, DMSO-d₆) δ: 199.1, 173.3, 156.7, 153.4, 151.8, 138.2, 137.6, 135.7, 134.9, 132.0, 128.5 (2C), 128.4, 127.6 (2C), 124.5 (2C), 124.1, 103.4, 101.3, 52.6, 51.2, 37.6, 37.5, 33.2. 28.1, 24.3, 23.5, 21.1; HRMS (ASAP+, m/z): found 485.2551, calcd for C₂₉H₃₃N₄O₃, [M+H]⁺, 485.2553; mp. 182-184° C.

### 6-(4-(4-(Methyl(3-methylbenzyl)amino)-7H-pyrrolo[2,3-d]pyrimidin6-yl)phenyl)tetrahydro-2H-pyran-2-one (LR-1-124)

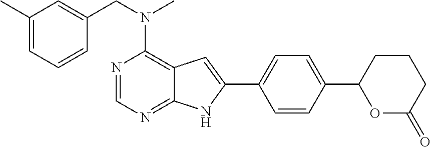 [SMILES: Cc1cccc(CN(C)c2ncnc3[nH]c(-c4ccc(C5CCCC(=O)O5)cc4)cc23)c1]

¹H-NMR (600 MHz, DMSO-d₆) δ: 12.19 (s, 1H), 8.15 (s, 1H), 7.88-7.87 (d, J=8.3 Hz, 2H), 7.42-7.41, (d, J=8.3 Hz, 2H), 7.22-7.20 (t, J=7.8 Hz, 1H), 7.10-7.05 (m, 4H), 5.45-5.43 (dd, J=10.7, 3.4, 1H), 5.01 (s, 2H), 3.36 (s, 3H), 2.65-2.61 (m, 1H), 2.53-2.52 (m, 1H), 2.27 (s, 3H), 2.10-2.06 (m, 1H), 1.99-1.90 (m, 1H), 1.87-1.79 (m, 2H); 13C NMR (150 MHz, DMSO-d₆) δ: 171.1, 156.5, 153.0, 151.3, 139.2, 138.4, 137.6, 132.8, 131.3, 128.4, 127.6 (2C), 126.5 (2C), 124.8 (2C), 124.1 103.3, 99.2, 80.4, 52.6, 37.4, 29.5, 29.1, 21.1, 18.1; HRMS (ASAP+, m/z): found 427.2129, calcd for C₂₆H₂₇N₄O₂, [M+H]⁺, 427.2134; mp. 220-223° C.

### 6-(4-(2-(2-(2-Methoxyethoxy)ethoxy)ethoxy)phenyl)-N-methyl-N-(3-methylbenzyl)-7H-pyrrolo[2,3-d]pyrimidin-4-amine (NOR-4-004)

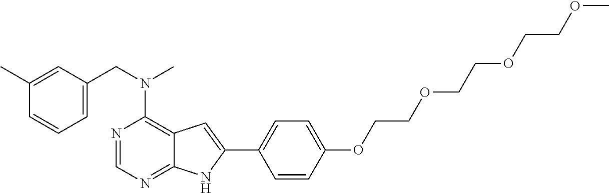

¹H NMR (400 MHz, DMSO-d₆), δ: 12.07 (s, 1H), 8.13 (s, 1H), 7.80-7.76 (m, 2H), 7.24-7.19 (m, 1H), 7.11-7.04 (m, 3H), 7.01-6.98 (m, 2H), 6.92 (s, 1H), 5.01 (s, 2H), 4.15-4.11 (m, 2H), 3.78-3.74 (m, 2H), 3.62-3.50 (m, 6H), 3.45-3.42 (m, 2H), 3.35 (s, 3H), 3.24 (s, 3H), 2.27 (s, 3H); ¹³C NMR (100 MHz, DMSO-d₆), δ: 159.6, 156.7, 153.4, 153.3, 138.9, 138.0, 135.2, 129.2 (2C), 128.9, 128.0, 126.6, 124.6, 115.2 (2C), 109.4, 103.7, 71.8 (C33), 70.4, 70.2, 70.1, 69.4, 67.7, 58.6, 53.2, 37.8, 21.6; HRMS (ASCI/ASAP, m/z): found 491.2657, calcd for C₂₈H₃₄N₄O₄, [M+H]⁺, 491.2658.

### 4-(4-(Methyl(3-methylbenzyl)amino)-7H-pyrrolo[2,3-d]pyrimidin-6-yl)benzenesulfonamide (FAB02-15)

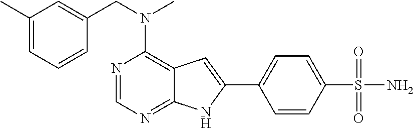 [SMILES: Cc1cccc(CN(C)c2ncnc3[nH]c(-c4ccc(S(N)(=O)=O)cc4)cc23)c1]

¹H NMR (600 MHz, DMSO-d₆) δ 12.36 (s, 1H), 8.20 (s, 1H), 8.08-8.04 (m, 2H), 7.87-7.83 (m, 2H), 7.38 (s, 2H), 7.29 (s, 1H), 7.24 (t, J=7.6 Hz, 1H), 7.14-7.06 (m, 3H), 5.05 (s, 2H), 3.40 (s, 3H), 2.30 (s, 3H); ¹³C NMR (151 MHz, DMSO-d₆) δ 156.7, 153.3, 151.8, 142.2, 138.2, 137.6, 134.7, 131.6, 128.4, 127.6 (2C), 126.1 (2C), 124.7 (2C), 124.2, 103.3, 101.1, 52.6, 39.5, 37.4, 21.1. HRMS (ES+, m/z): found 408.1495 (calcd. C₂₁H₂₁N₅O₂S, [M+H]⁺ 408.1415

### 6-(4-(Difluoromethyl)phenyl)-N-methyl-N-(3-methylbenzyl)-7H-pyrrolo[2,3-d]pyrimidin-4-amine (FAB02-13)

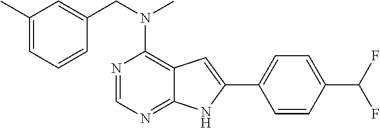 [SMILES: Cc1cccc(CN(C)c2ncnc3[nH]c(-c4ccc(C(F)F)cc4)cc23)c1]

¹H NMR (600 MHz, DMSO-d₆) δ 12.31 (s, 1H), 8.19 (s, 1H), 8.01 (d, J=8.1 Hz, 2H), 7.62 (d, J=8.1 Hz, 2H), 7.26-7.19 (m, 2H), 7.16-6.90 (m, 4H), 5.04 (s, 2H), 3.39 (s, 3H), 2.29 (s, 3H); ¹³C NMR (151 MHz, DMSO-d₆) δ 156.6, 153.2, 151.6, 138.3, 137.60, 133.9, 132.4 (t, J=22 Hz) 132.1, 128.4, 127.6 (2C), 126.2 (t, J=7 Hz, 2C), 124.9 (2C), 124.1, 114.9 (t, J=240 Hz), 103.3, 100.4, 52.6, 39.5, 37.4, 21.0; HRMS (ES+, m/z): found 379.1740 (calcd. C₂₂H₂₀F₂N₄, [M+H]⁺ 379.1656

### Methyl 3-(4-(methyl(3-methylbenzyl)amino)-7H-pyrrolo[2,3-d]pyrimidin-6-yl)benzoate (FAB01-23)

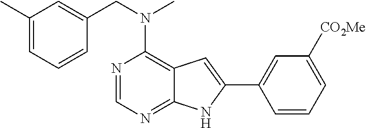 [SMILES: COC(=O)c1cccc(-c2cc3c(N(C)Cc4cccc(C)c4)ncnc3[nH]2)c1]

¹H NMR (600 MHz, DMSO-d₆) δ 12.35 (s, 1H), 8.43 (s, 1H), 8.16 (s, 1H), 8.12 (dt, J=7.8, 1.4 Hz, 1H), 7.85 (dt, J=7.8, 1.3 Hz, 1H), 7.57 (t, J=7.8 Hz, 1H), 7.21 (t, J=7.6 Hz, 1H), 7.16 (s, 1H), 7.12-7.04 (m, 3H), 5.02 (s, 2H), 3.89 (s, 3H), 3.37 (s, 3H), 2.27 (s, 3H). ¹³C NMR (151 MHz, DMSO-d₆) δ 166.1, 156.6, 153.2, 151.5, 138.4, 137.6, 132.2, 132.1, 130.4, 129.3 (2C), 128.4, 127.7, 127.6, 125.2, 124.1, 103.2, 99.9, 52.6, 52.2, 39.5, 37.4, 21.1. HRMS (ES+, m/z): found 387.1826 (calcd. C₂₃H₂₂N₄O₂, [M+H]⁺ 387.1742.

### 3-(4-(Methyl(3-methylbenzyl)amino)-7H-pyrrolo[2,3-d]pyrimidin-6-yl)benzoic acid (FAB01-25)

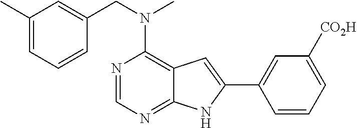 [SMILES: Cc1cccc(CN(C)c2ncnc3[nH]c(-c4cccc(C(=O)O)c4)cc23)c1]

¹H NMR (600 MHz, DMSO-d₆) δ 13.10 (s, 1H), 12.39 (s, 1H), 8.45-8.41 (m, 1H), 8.19 (s, 1H), 8.13-8.08 (m, 1H), 7.88-7.83 (m, 1H), 7.56 (t, J=7.8 Hz, 1H), 7.26-7.20 (m, 1H), 7.17 (s, 1H), 7.12 (s, 1H), 7.11-7.06 (m, 2H), 5.04 (s, 2H), 3.40 (s, 3H), 2.29 (s, 3H). ¹³C NMR (151 MHz, DMSO) δ 167.2, 156.4, 152.8, 151.1, 138.2, 137.6, 132.4, 131.9, 131.5, 129.1, 128.9, 128.4, 127.9, 127.6 (2C), 125.5, 124.1, 103.2, 99.9, 52.7, 39.5, 37.5, 21.1; HRMS (ES+, m/z): found 373.1667 (calcd. C₂₂H₂₀N₄O₂, [M+H]⁺, 373.1742

### 3-(4-(4-(Methyl(3-methylbenzyl)amino)-7H-pyrrolo[2,3-d]pyrimidin-6-yl)phenyl)oxetan-3-ol (LR-2-060-1)

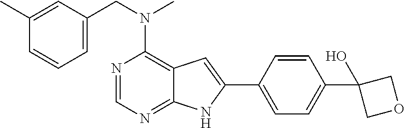 [SMILES: Cc1cccc(CN(C)c2ncnc3[nH]c(-c4ccc(C5(O)COC5)cc4)cc23)c1]

¹H NMR (600 MHz, DMSO-d₆) δ 12.19 (s, 1H), 8.15 (s, 1H), 7.88 (d, J=8.5 Hz, 2H), 7.62 (d, J=8.5 Hz, 2H), 7.21 (t, J=7.6 Hz, 1H), 7.09 (br s, 1H), 7.08-7.04 (m, 3H), 6.37 (br s, 1H), 5.01 (s, 2H), 4.77 (d, J=6.8 Hz, 2H), 4.70 (d, J=6.8 Hz, 2H), 3.36 (s, 3H), 2.27 (s, 3H); ¹³C NMR (151 MHz, DMSO-d₆) δ 156.5, 153.0, 151.2, 143.1, 138.4, 137.6, 133.0, 130.4, 128.4, 127.6 (2C), 125.0 (2C), 124.6 (2C), 124.1, 103.3, 98.9, 85.2 (2C), 73.9, 52.6, 37.4, 21.1; IR (neat, cm⁻¹): 3369, 3105, 2872, 1569, 1509, 1417, 1318, 1072, 973, 930, 834, 777, 694, 544; HRMS (ES+, m/z): 401.1984 (calcd. C₂₄H₂₅N₄O₂, 401.1978 [M+H]⁺)

### 2-(4-(4-(Methyl(3-methylbenzyl)amino)-7H-pyrrolo[2,3-d]pyrimidin-6-yl)phenyl)propane-1,2,3-triol (LR-2-060-2)

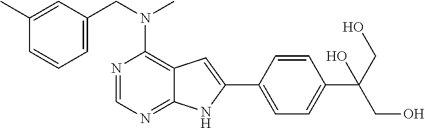 [SMILES: Cc1cccc(CN(C)c2ncnc3[nH]c(-c4ccc(C(O)(CO)CO)cc4)cc23)c1]

¹H NMR (600 MHz, DMSO-d₆) δ 12.13 (s, 1H), 8.14 (s, 1H), 7.75 (d, J=8.5 Hz, 2H), 7.50 (d, J=8.5 Hz, 2H), 7.21 (t, J=7.6 Hz, 1H), 7.09 (br s, 1H), 7.07-7.04 (m, 2H), 6.99 (br s, 1H), 5.01 (s, 2H), 4.76 (s, 1H), 4.60-4.57 (m, 2H), 3.60-3.57 (m, 4H), 3.35 (s, 3H), 2.27 (s, 3H); ¹³C NMR (151 MHz, DMSO-d₆) δ 156.4, 152.9, 151.0, 143.8, 138.4, 137.6, 133.5, 129.5, 128.4, 127.6 (2C), 126.6 (2C), 124.1, 123.9 (2C), 103.3, 98.3, 76.1, 66.1 (2C), 52.6, 37.3, 21.1; IR (neat, cm⁻¹): 3404, 3139, 2920, 2851, 1738, 1575, 1442, 1375, 1180, 1054, 938, 769; HRMS (ASAP+, m/z): 419.2077 (calcd. C₂₄H₂₇N₄O₃, 419.2083 [M+H]⁺)

### Dimethyl(4-(4-(methyl(3-methylbenzyl)amino)-7H-pyrrolo[2,3-d]pyrimidin-6-yl)phenyl)phosphine oxide (LR-2-116)

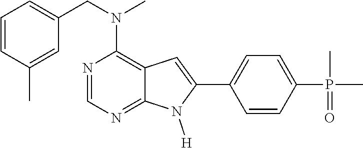 [SMILES: Cc1cccc(CN(C)c2ncnc3[nH]c(-c4ccc(P(C)(C)=O)cc4)cc23)c1]

¹H NMR (600 MHz, DMSO-d₆) δ 12.30 (s, 1H), 8.17 (s, 1H), 7.99 (dd, J=8.5, 2.1 Hz, 2H), 7.80-7.76 (m, 2H), 7.22-7.19 (m, 2H), 7.10 (s, 1H), 7.08-7.04 (m, 2H), 5.02 (s, 2H), 3.37 (s, 3H), 2.27 (s, 3H), 1.66 (d, J=13.3 Hz, 6H); ¹³C NMR (151 MHz, DMSO-d₆) δ 156.6, 153.2, 151.6, 138.3, 137.6, 134.3 (d, J=96.9 Hz), 134.1 (d, J=2.6 Hz), 132.2, 130.3 (d, J=9.9 Hz, 2C), 128.5, 127.6 (2C), 124.4 (d, J=11.4 Hz, 2C), 124.2, 103.3, 100.5, 52.6, 37.5, 21.1, 17.8 (d, J=70.5 Hz, 2C); ³¹P NMR (243 MHz, DMSO-d₆) δ 33.2; IR (neat, cm⁻¹): 3085, 2971, 1566, 1413, 1300, 1166, 1064, 827, 761; HRMS (ASAP+, m z): found 405.1840, calcd. for C₂₃H26N40P, [M+H]⁺, 405.1844.

### 6-(1-Isopropyl-1H-pyrazol-4-yl)-N-methyl-N-(3-methylbenzyl)-7H-pyrrolo[2,3-d]pyrimidin-4-amine (LR-2-108)

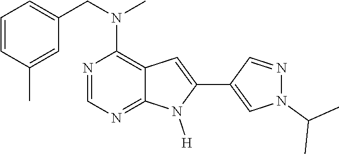 [SMILES: Cc1cccc(CN(C)c2ncnc3[nH]c(-c4cnn(C(C)C)c4)cc23)c1]

¹H NMR (600 MHz, DMSO-d₆) δ 11.91 (s, 1H), 8.17 (s, 1H), 8.09 (s, 1H), 7.88 (s, 1H), 7.20 (t, J=7.5 Hz, 1H), 7.10-7.02 (m, 3H), 6.72 (s, 1H), 4.97 (s, 2H), 4.49 (hept, J=6.6 Hz, 1H), 3.30 (s, 3H), 2.27 (s, 3H), 1.43 (d, J=6.6 Hz, 6H); ¹³C NMR (151 MHz, DMSO-d₆) δ 156.0, 152.1, 150.3, 138.5, 137.6, 135.6, 128.4, 127.6, 127.6, 127.3, 124.4, 124.2, 113.9, 103.0, 96.2, 53.1, 52.5, 37.1, 22.6 (2C), 21.1; IR (neat, cm⁻¹): 3107, 2973, 1566, 1506, 1409, 1337, 1072, 936, 764, 694; HRMS (ES+, m z): found 361.2141, calcd for C₂₁H₂₅N6, [M+H]⁺, 361.2141.

### 6-(4-(1H-Tetrazol-5-yl)phenyl)-N-methyl-N-(3-methylbenzyl)-7H-pyrrolo[2,3-d]pyrimidin-4-amine (FAB 02-14)

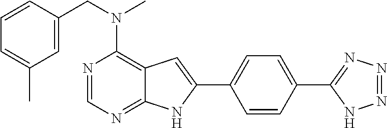 [SMILES: Cc1cccc(CN(C)c2ncnc3[nH]c(-c4ccc(-c5nnn[nH]5)cc4)cc23)c1]

¹H NMR (600 MHz, DMSO-d₆) δ 12.76 (s, 1H), 8.28 (s, 1H), 8.11 (m, 4H), 7.39 (s, 1H), 7.26 (t, J=7.6 Hz, 1H), 7.15 (s, 1H), 7.11 (m, 3H), 5.09 (s, 2H), 3.44 (s, 3H), 2.30 (s, 3H); HRMS (ES+, m/z): found 397.1888, calcd for C₂₂H₂₁N₈, [M+H]⁺, 397.1889.

### Spectroscopic Data Pyran Substituted Pyrrolopyrimidines

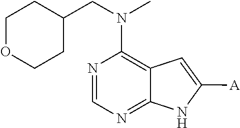

### N-Methyl-6-phenyl-N-((tetrahydro-2H-pyran-4-yl)methyl)-7H-pyrrolo[2,3-d]pyrimidin-4-amine (SH01-18)

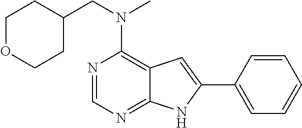 [SMILES: CN(CC1CCOCC1)c1ncnc2[nH]c(-c3ccccc3)cc12]

¹HNMR (600 MHz, DMSO-d₆) δ: 12.13 (s, 1H), 8.11 (s, 1H), 7.88 (d, J=8.2 Hz, 2H), 7.42 (t, J=7.6 Hz, 2H), 7.28 (t, J=7.4 Hz, 1H), 7.07 (s, 1H), 3.86-3.81 (m, 2H), 3.69 (d, J=7.4 Hz, 2H), 3.40 (s, 3H), 3.28-3.21 (m, 2H), 2.11-2.02 (m, 1H), 1.58-1.51 (m, 2H), 1.35-1.25 (m, 2H); ¹³CNMR (150 MHz, DMSO-d₆) δ: 156.4, 152.8, 151.1, 132.9, 131.6, 128.8 (2C), 127.2, 124.7 (2C), 103.3, 98.9, 66.8 (2C), 55.4, 38.9, 33.9, 30.3 (2C); HRMS (ASAP+, m/z): found 323.1875, calcd for C₁₉H₂₃N₄O, [M+H]⁺, 323.1872; mp. >235° C. (decomp).

### (4-(4-(Methyl((tetrahydro-2H-pyran-4-yl)methyl)amino)-7H-pyrrolo[2,3-d]pyrimidin-6-yl)phenyl)methanol (TIA05-028)

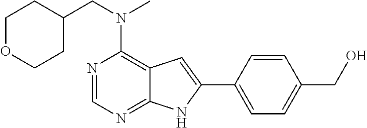 [SMILES: CN(CC1CCOCC1)c1ncnc2[nH]c(-c3ccc(CO)cc3)cc12]

¹H NMR (400 MHz, DMSO-d₆) δ: 12.08 (s, 1H), 8.10 (s, 1H), 7.86-7.80 (m, 2H), 7.39-7.32 (m, 2H), 7.04 (s, 1H), 5.20 (t, J=5.7 Hz, 1H), 4.51 (d, J=5.7 Hz, 2H), 3.87-3.79 (m, 2H), 3.69 (d, J=7.4 Hz, 2H), 3.40 (s, 3H), 3.30-3.19 (m, 2H), 2.11-2.01 (m, 1H), 1.57-1.50 (m, 2H), 1.37-1.22 (m, 2H); ¹³C NMR (101 MHz, DMSO-d₆) δ: 156.4, 152.8, 151.0, 141.6, 133.0, 130.1, 126.9 (2C), 124.5 (2C), 103.3, 98.6, 66.8 (2C), 62.6, 55.4, 39.0, 34.0, 30.3 (2C). HRMS (ASAP+, m/z): found 353.1975, calcd for C₂₀H₂₅N₄O₂, [M+H]⁺, 353.1978.

### 2-(4-(4-(Methyl((tetrahydro-2H-pyran-4-yl)methyl)amino)-7H-pyrrolo[2,3-d]pyrimidin-6-yl)phenyl)ethan-1-ol (TIA05-032)

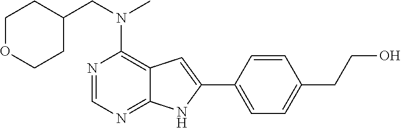 [SMILES: CN(CC1CCOCC1)c1ncnc2[nH]c(-c3ccc(CCO)cc3)cc12]

¹H NMR (600 MHz, DMSO-d₆) δ 12.06 (s, 1H), 8.09 (s, 1H), 7.80-7.75 (m, 2H), 7.28-7.24 (m, 2H), 7.00 (s, 1H), 4.66 (t, J=5.2 Hz, 1H), 3.86-3.80 (m, 2H), 3.68 (d, J=7.4 Hz, 2H), 3.62 (td, J=7.0, 5.1 Hz, 2H), 3.39 (s, 3H), 3.28-3.21 (m, 2H), 2.73 (t, J=7.0 Hz, 2H), 2.11-2.01 (m, 1H), 1.57-1.51 (m, 2H), 1.34-1.22 (m, 2H); ¹³C NMR (151 MHz, DMSO-d₆) δ 156.3, 152.7, 150.9, 138.8, 133.1, 129.3 (3C), 124.6 (2C), 103.3, 98.3, 66.8 (2C), 62.1, 55.4, 38.9, 38.7, 34.0, 30.3 (2C); HRMS (ESI+, m/z): found 367.2136, calcd for C₂₁H₂₇N₄O₂, [M+H]⁺, 367.2134.

### 6-(4-Methoxyphenyl)-N-methyl-N-((tetrahydro-2H-pyran-4-yl)methyl)-7H-pyrrolo[2,3-d]pyrimidin-4-amine (SH-01-17)

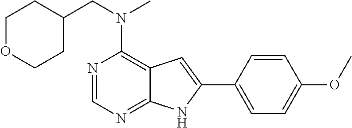 [SMILES: COc1ccc(-c2cc3c(N(C)CC4CCOCC4)ncnc3[nH]2)cc1]

¹H NMR (600 MHz, DMSO-d₆) δ: 12.02 (s, 1H), 8.09 (s, 1H), 7.81 (d, J=8.8 Hz, 2H), 6.99 (d, J=8.9 Hz, 2H), 6.93 (s, 1H), 3.86-3.81 (m, 2H), 3.79 (s, 3H), 3.68 (d, J=7.4 Hz, 2H), 3.39 (s, 3H), 3.24 (ap. td, J=11.9, 1.9 Hz, 2H), 2.10-2.01 (m, 1H), 1.57-1.51 (m, 2H), 1.34-1.24 (m, 2H); ¹³C NMR (150 MHz, DMSO-d₆) δ: 158.6, 156.2, 152.6, 150.6, 133.1, 126.9 (2C), 124.2, 114.2 (2C), 103.1, 97.4, 66.7 (2C), 55.3, 55.2, 38.9, 33.9, 30.3 (2C); HRMS (ASAP+, m/z): found 353.1978, calcd for C₂₀H₂₅N₄O₂, [M+H]⁺, 353.1978; mp. 220-224° C.

### Methyl 4-(4-(methyl((tetrahydro-2H-pyran-4-yl)methyl)amino)-7H-pyrrolo[2,3-d]pyrimidin-6-yl)benzoate (SH-01-26)

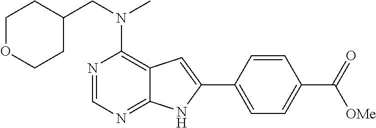 [SMILES: COC(=O)c1ccc(-c2cc3c(N(C)CC4CCOCC4)ncnc3[nH]2)cc1]

¹H NMR (600 MHz, DMSO-d₆) δ: 12.29 (s, 1H), 8.13 (s, 1H), 8.03 (d, J=8.6 Hz, 2H), 7.98 (d, J=8.6 Hz, 2H), 7.29 (s, 1H), 3.86 (s, 3H), 3.86-3.81 (m, 2H), 3.70 (d, J=7.4 Hz, 2H), 3.42 (s, 3H), 3.28-3-22 (m, 2H), 2.12-2.01 (m, 1H), 1.57-1.51 (m, 2H), 1.35-1.25 (m, 2H); ¹³C NMR (150 MHz, DMSO-d₆) δ: 165.9, 156.6, 153.2, 151.7, 136.0, 131.5, 129.6 (2C), 127.6, 124.5 (2C), 103.4, 101.5, 66.8 (2C), 55.3, 52.1, 39.0, 33.9, 30.3 (2C); HRMS (ASAP+, m/z): found 381.1927, calcd for C₂₁H₂₅N₄O₃, [M+H]⁺; mp. >238° C. (decomp.).

### N-Methyl-6-(4-(morpholinomethyl)phenyl)-N-((tetrahydro-2H-pyran-4-yl)methyl)-7H-pyrrolo[2,3-d]pyrimidin-4-amine (TIA05-030)

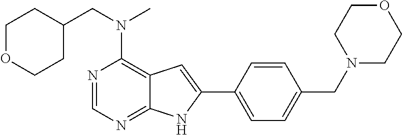 [SMILES: CN(CC1CCOCC1)c1ncnc2[nH]c(-c3ccc(CN4CCOCC4)cc3)cc12]

¹H NMR (600 MHz, DMSO-d₆) δ: 12.08 (s, 1H), 8.10 (s, 1H), 7.85-7.80 (m, 2H), 7.36-7.32 (m, 2H), 7.03 (s, 1H), 3.86-3.80 (m, 2H), 3.69 (d, J=7.4 Hz, 2H), 3.60-3.55 (m, 4H), 3.47 (s, 2H), 3.39 (s, 3H), 3.28-3.21 (m, 2H), 2.38-2.34 (m, 4H), 2.11-2.02 (m, 1H), 1.57-1.51 (m, 2H), 1.34-1.25 (m, 2H); ¹³C NMR (151 MHz, DMSO-d₆) δ: 156.4, 152.8, 151.0, 136.9, 132.9, 130.4, 129.4 (2C), 124.6 (2C), 103.3, 98.7, 66.8 (2C), 66.2 (2C), 62.1, 55.4, 53.2 (2C), 38.9, 34.0, 30.3 (2C); HRMS (ASAP+, m/z): found 422.2549, calcd for C₂₄H₃₂N₅O₂, [M+H]⁺, 422.2556.

### 6-(4-Fluorophenyl)-N-methyl-N-((tetrahydro-2H-pyran-4-yl)methyl)-7H-pyrrolo[2,3-d]pyrimidin-4-amine (SH-01-27)

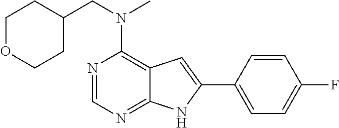 [SMILES: CN(CC1CCOCC1)c1ncnc2[nH]c(-c3ccc(F)cc3)cc12]

¹H NMR (600 MHz, DMSO-d₆) δ: 12.12 (s, 1H), 8.11 (s, 1H), 7.94-7-89 (m, 2H), 7.29-7.24 (m, 2H), 7.05 (s, 1H), 3.86-3.90 (m, 2H), 3.68 (d, J=7.4 Hz, 2H), 3.39 (s, 3H), 3.28-3-21 (m, 2H), 2.11-2.01 (m, 1H), 1.57-1.50 (m, 2H), 1.34-1.24 (m, 2H); ¹³C NMR (150 MHz, DMSO-de) 6:161.5 (d, J=244.7 Hz), 156.4, 152.9, 151.1, 132.0, 128.3 (d, J=3.2 Hz), 126.72 (d, J=8.0 Hz, 2C), 115.7 (d, J=21.3 Hz, 2C), 103.3, 98.9, 66.8 (2C), 55.1 (d, J=64.3 Hz), 38.9, 33.9, 30.3 (2C); ¹⁹F NMR (565 MHz, DMSO-d₆, C₆F₆) δ: −114.9; HRMS (ASAP+, m/z): found 341.1776, calcd for C₁₉H₂₂N₄OF, [M+H]⁺, 341.1778; mp. >237° C. (decomp.).

### N-Methyl-N-((tetrahydro-2H-pyran-4-yl)methyl)-6-(4-(trifluoromethyl)phenyl)-7H-pyrrolo[2,3-d]pyrimidin-4-amine (SH-01-45-P1)

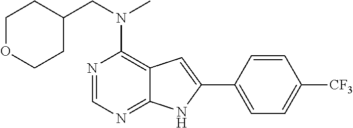 [SMILES: CN(CC1CCOCC1)c1ncnc2[nH]c(-c3ccc(C(F)(F)F)cc3)cc12]

¹H NMR (600 MHz, DMSO-d₆) δ: 12.31 (s, 1H), 8.14 (s, 1H), 8.10 (d, J=8.1 Hz, 2H), 7.77 (d, J=8.2 Hz, 2H), 7.29 (s, 1H), 3.86-3.80 (m, 2H), 3.71 (d, J=7.4 Hz, 2H), 3.42 (s, 3H), 3.25 (td, J=11.7, 2.1 Hz, 2H), 2.12-2.02 (m, 1H), 1.57-1.51 (m, 2H), 1.36-1.24 (m, 2H); ¹³C NMR (150 MHz, DMSO-d₆) δ: 156.6, 153.2, 151.8, 135.5, 131.2, 126.9 (q, J=31.6 Hz, 1C), 125.7 (q, J=3.3 Hz, 2C) 125.2, 125.0, 123.4 (q, J=271.4 Hz, 1C), 103.3, 101.3, 66.7 (2C), 55.3, 39.0, 33.9, 30.3 (2C); ¹⁹F-NMR (565 MHz, DMSO-d₆, C₆F₆) δ: −75.8; HRMS (ASAP+, m/z): found 391.1750, calcd for C₂₀H₂₂N₄OF₃, [M+H]⁺, 391.1746.

### 6-(4-(Difluoromethyl)phenyl)-N-methyl-N-((tetrahydro-2H-pyran-4-yl)methyl)-7H-pyrrolo[2,3-d]pyrimidin-4-amine (SH-01-58)

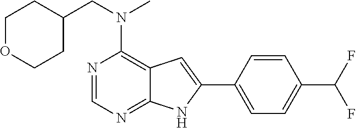 [SMILES: CN(CC1CCOCC1)c1ncnc2[nH]c(-c3ccc(C(F)F)cc3)cc12]

¹H NMR (600 MHz, DMSO-d₆) δ 12.23 (s, 1H), 8.13 (s, 1H), 8.02 (d, J=8.1 Hz, 2H), 7.61 (d, J=7.9 Hz, 2H), 7.21 (s, 1H), 7.04 (t, JF=56.4 Hz, 1H), 3.86-3.80 (m, 2H), 3.70 (d, J=7.4 Hz, 2H), 3.41 (s, 3H), 3.25 (td, J=11.7, 2.1 Hz, 2H), 2.10-2.03 (m, 1H), 1.57-1.51 (m, 2H), 1.30 (qd, J=12.3, 4.5 Hz, 2H); ¹³C NMR (150 MHz, DMSO-d₆) δ: 156.6, 153.1, 151.5, 134.1, 132.5 (t, J=21.9 Hz), 131.8, 126.2 (t, J=6.0 Hz, 2C), 124.9, 114.9 (t, J=235.5 Hz, 2C), 103.3, 100.4, 66. (2C), 55.3, 39.0, 33.9, 30.3 (2C); ¹⁹FNMR (565 MHz, DMSO-d₆, C₆F₆) 8: −111.5; HRMS (ASAP+, m/z): found 373.1841, calcd for C₂₀H₂₃N₄OF₂, [M+H]⁺, 373.1840.

### 4-(4-(Methyl((tetrahydro-2H-pyran-4-yl)methyl)amino)-7H-pyrrolo[2,3-d]pyrimidin-6-yl)benzoic acid (SH-01-30-P1)

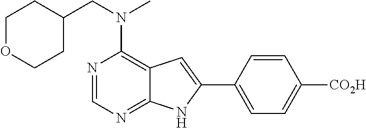 [SMILES: CN(CC1CCOCC1)c1ncnc2[nH]c(-c3ccc(C(=O)O)cc3)cc12]

¹H NMR (600 MHz, DMSO-d₆) δ: 12.89 (s, 1H), 12.27 (s, 1H), 8.13 (s, 1H), 8.01 (d, J=8.5 Hz, 2H), 7.95 (d, J=8.5 Hz, 2H), 7.26 (s, 1H), 3.86-3.80 (dd, J=8.3, 2.5 Hz, 2H), 3.70 (d, J=7.4 Hz, 2H), 3.42 (s, 3H), 3.25 (td, J=11.7, 2.1 Hz, 2H), 2.11-2.02 (m, 1H), 1.57-1.51 (m, 2H), 1.30 (qd, J=12.1, 4.4 Hz, 2H); ¹³C NMR (150 MHz, DMSO-d₆) δ: 167.0, 156.6, 153.2, 151.6, 135.7, 131.8, 129.8 (2C), 128.9, 124.5 (2C), 103.4, 101.2, 66.8 (2C), 55.4, 38.9, 33.9, 30.3 (2C); HRMS (ASAP+, m/z): found 367.1771, calcd for C₂₀H₂₃N₄O₃, [M+H]⁺, 367.1770.

### 4-(4-(Methyl((tetrahydro-2H-pyran-4-yl)methyl)amino)-7H-pyrrolo[2,3-d]pyrimidin-6-yl)benzenesulfonamide

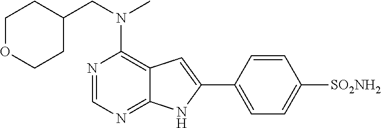 [SMILES: CN(CC1CCOCC1)c1ncnc2[nH]c(-c3ccc(S(N)(=O)=O)cc3)cc12]

¹H NMR (600 MHz, DMSO-d₆) δ: 12.27 (s, 1H), 8.14 (s, 1H), 8.06 (d, J=8.6 Hz, 2H), 7.83 (d, J=8.6 Hz, 2H), 7.35 (s, 2H), 7.27 (s, 1H), 3.86-3.80 (m, 2H), 3.70 (d, J=7.4 Hz, 2H), 3.42 (s, 3H), 3.25 (td, J=11.7, 2.1 Hz, 2H), 2.12-2.02 (m, 1H), 1.57-1.51 (m, 2H), 1.30 (qd, J=12.4, 4.4 Hz, 2H); ¹³C NMR (150 MHz, DMSO-d₆) δ: 156.6, 153.2, 151.7, 142.1, 134.7, 131.4, 126.2 (2C), 124.7 (2C), 103.4, 101.2, 66.8 (2C), 55.3, 39.0, 33.9, 30.2 (2C); HRMS (ASAP+, m/z): found 402.1602, calcd for C₁₉H₂₄N₅O₃S, [M+H]⁺, 402.1600.

### N-Methyl-6-(pyridin-3-yl)-N-((tetrahydro-2H-pyran-4-yl)methyl)-7H-pyrrolo[2,3-d]pyrimidin-4-amine (SH-01-39-P1)

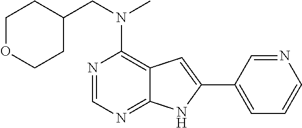 [SMILES: CN(CC1CCOCC1)c1ncnc2[nH]c(-c3cccnc3)cc12]

¹H NMR (600 MHz, DMSO-d₆) δ:12.27 (s, 1H), 9.14-9.10 (m, 1H), 8.47 (dd, J=4.7, 1.6 Hz, 1H), 8.27-8.18 (m, 1H), 8.13 (s, 1H), 7.44 (ddd, J=8.0, 4.8, 0.9 Hz, 1H), 7.24 (s, 1H), 3.86-3.80 (m, 2H), 3.70 (d, J=7.4 Hz, 2H), 3.41 (s, 3H), 3.25 (td, J=11.7, 2.1 Hz, 2H), 2.10-2.03 (m, 1H), 1.57-1.51 (m, 2H), 1.30 (qd, J=12.3, 4.5 Hz, 2H); ¹³C NMR (150 MHz, DMSO-d₆) δ: 156.5, 153.1, 151.5, 147.9, 146.0, 131.6, 129.8, 127.6, 123.7, 103.2, 100.3, 66.8 (2C), 55.3, 39.0, 33.9, 30.3 (2C); HRMS (ASAP+, m/z): found 324.1825, calcd for C₁₈H₂₂N₅O, [M+H]⁺, 324.1824.

### (3-(4-(Methyl((tetrahydro-2H-pyran-4-yl)methyl)amino)-7H-pyrrolo[2,3-d]pyrimidin-6-yl)phenyl)methanol (SH-01-44-P1)

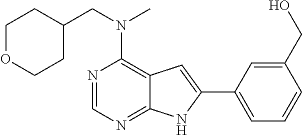 [SMILES: CN(CC1CCOCC1)c1ncnc2[nH]c(-c3cccc(CO)c3)cc12]

¹H NMR (600 MHz, DMSO-d₆) δ: 12.10 (s, 1H), 8.10 (s, 1H), 7.84-7.80 (m, 1H), 7.76-7.71 (ap. d, J=7.2 Hz, 1H), 7.38 (t, J=7.7 Hz, 1H), 7.25 (d, J=6.8 Hz, 1H), 7.05 (ap s, 1H), 5.23 (t, J=5.7 Hz, 1H), 4.54 (d, J=5.7 Hz, 2H), 3.87-3.81 (m, 2H), 3.69 (d, J=7.4 Hz, 2H), 3.41 (s, 3H), 3.25 (td, J=11.7, 2.1 Hz, 2H), 2.12-2.01 (m, 1H), 1.57-1.51 (m, 2H), 1.27 (dd, J=12.5, 4.0 Hz, 2H); ¹³C NMR (150 MHz, DMSO-d₆) δ: 156.4, 152.8, 151.1, 143.1, 133.1, 131.3, 128.6, 125.5, 123.1, 122.9, 103.3, 98.8, 66.7 (2C), 62.9, 55.3, 38.9, 33.9, 30.3 (2C): HRMS (ASAP+, m/z): found 353.1980, calcd for C₂₀H₂₅N₄O₂ [M+H]⁺ 353.1978.

### Methyl 3-(4-(methyl((tetrahydro-2H-pyran-4-yl)methyl)amino)-7H-pyrrolo[2,3-d]pyrimidin-6-yl)benzoate (SH-01-43-P1)

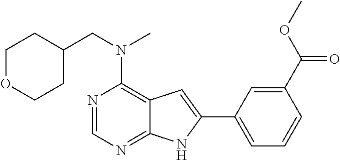 [SMILES: COC(=O)c1cccc(-c2cc3c(N(C)CC4CCOCC4)ncnc3[nH]2)c1]

¹H NMR (600 MHz, DMSO-d₆) δ: 12.29 (s, 1H), 8.48-8.44 (m, 1H), 8.16-8.13 (m, 1H), 8.12 (s, 1H), 7.87-8.83 (m, 1H), 7.57 (t, J=7.8 Hz, 1H), 7.18 (s, 1H), 3.90 (s, 3H), 3.87-3.81 (m, 2H), 3.70 (d, J=7.4 Hz, 2H), 3.42 (s, 3H), 3.25 (td, J=11.7, 2.1 Hz, 2H), 2.10-2.01 (m, 1H), 1.56-1.51 (m, 2H), 1.30 (qd, J=12.1, 4.5 Hz, 2H); ¹³C NMR (150 MHz, DMSO-d₆) δ: 166.2, 156.6, 153.1, 151.4, 132.2, 131.8, 130.4, 129.3, 127.7, 125.2, 103.3, 100.0, 66.8 (2C), 55.3, 52.3, 38.9, 33.9, 30.3 (2C); HRMS (ASAP+, m/z): found 381.1924, calcd for C₂₁H₂₅N₄O₃, [M+H]⁺, 381.1927

### N-Methyl-6-(3-nitrophenyl)-N-((tetrahydro-2H-pyran-4-yl)methyl)-7H-pyrrolo[2,3-d]pyrimidin-4-amine (SH-01-50)

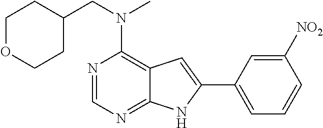 [SMILES: CN(CC1CCOCC1)c1ncnc2[nH]c(-c3cccc([N+](=O)[O-])c3)cc12]

¹H NMR (600 MHz, DMSO-d₆) δ:12.40 (s, 1H), 8.77 (t, J=2.0 Hz, 1H), 8.36-8.32 (m, 1H), 8.14 (s, 1H), 8.10 (ddd, J=8.2, 2.2, 0.9 Hz, 1H), 7.71 (t, J=8.0 Hz, 1H), 7.38 (s, 1H), 3.87-3.81 (m, 2H), 3.71 (d, J=7.4 Hz, 2H), 3.43 (s, 3H), 3.25 (td, J=11.7, 2.1 Hz, 2H), 2.12-2.02 (m, 1H), 1.56-1.51 (m, 2H), 1.30 (qd, J=12.3, 4.5 Hz, 2H); ¹³C NMR (150 MHz, DMSO-d₆) δ: 156.7, 153.2, 151.8, 148.6, 133.4, 130.8, 130.6, 130.3, 121.4, 118.9, 103.3, 101.4, 66.8 (2C), 55.3, 38.9, 33.8, 30.3 (2C); HRMS (ASAP+, m/z): found 368.1725, calcd for C₁₉H₂₂N₅O₃ [M+H]⁺ 368.1723.

### 6-(3-Aminophenyl)-N-methyl-N-((tetrahydro-2H-pyran-4-yl)methyl)-7H-pyrrolo[2,3-d]pyrimidin-4-amine (SH-01-53)

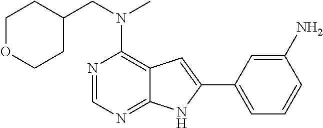 [SMILES: CN(CC1CCOCC1)c1ncnc2[nH]c(-c3cccc(N)c3)cc12]

¹H NMR (600 MHz, DMSO-d₆) δ: 11.97 (s, 1H), 8.09 (s, 1H), 7.06 (t, J=8.0 Hz, 1H), 7.02-6.97 (m, 2H), 6.83 (s, 1H), 6.56-6.45 (m, 1H), 5.08 (s, 2H), 3.87-3.81 (m, 2H), 3.67 (d, J=7.3 Hz, 2H), 3.38 (s, 3H), 3.25 (td, J=11.7, 2.1 Hz, 2H), 2.13-1.98 (m, 1H), 1.57-1.52 (m, 2H), 1.29 (qd, J=11.7, 4.5 Hz, 2H); ¹³C NMR (150 MHz, DMSO-d₆) δ: 156.3, 152.6, 150.8, 148.8, 134.0, 132.0, 129.3, 113.4, 112.9, 110.2, 103.2, 97.9, 66.7 (2C), 55.4, 38.9, 33.9, 30.3 (2C); HRMS (ASAP+, m/z): found 338.1998, calcd for C₁₉H₂₄N₅O, [M+H]⁺, 338.1981.

### 4-(4-(Methyl((tetrahydro-2H-pyran-4-yl)methyl)amino)-7H-pyrrolo[2,3-d]pyrimidin-6-yl)phenol (FAB 02-55)

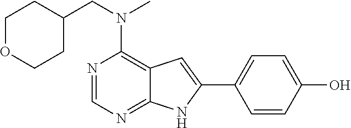 [SMILES: CN(CC1CCOCC1)c1ncnc2[nH]c(-c3ccc(O)cc3)cc12]

¹H NMR (600 MHz, DMSO-d₆) δ: 11.93 (s, 1H), 9.58 (s, 1H), 8.07 (s, 1H), 7.71-7.66 (m, 2H), 6.84 (s, 1H), 6.83-6.78 (m, 2H), 3.84-3.81 (m, 2H), 3.66 (d, J=16.7, 2H), 3.38 (s, 3H), 3.25 (td, J=11.7, 2.1 Hz, 2H), 2.11-2.01 (m, 1H), 1.54-1.51 (m, 2H), 1.30-1.24 (m, 2H). ¹³C NMR (151 MHz, DMSO-d₆) δ: 156.9, 156.1, 152.6, 150.5, 133.6, 126.2 (2C), 122.7, 115.6 (2C), 103.3, 96.7, 66.8 (2C), 55.3, 39.5, 38.8, 34.0, 30.3 (2C). HRMS (ASAP+, m/z): found 339.1823 calcd for C₁₉H₂₃N₄O₂, [M+H]⁺, 339.4124.

### 6-(Cyclohex-1-en-1-yl)-N-methyl-N-((tetrahydro-2H-pyran-4-yl)methyl)-7H-pyrrolo[2,3-d]pyrimidin-4-amine (FAB 02-61)

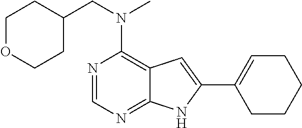 [SMILES: CN(CC1CCOCC1)c1ncnc2[nH]c(C3=CCCCC3)cc12]

¹H NMR (600 MHz, DMSO-d₆) δ: 11.64 (s, 1H), 8.06 (s, 1H), 6.46 (s, 1H), 6.40-6.35 (m, 1H), 3.83 (ddd, J=11.4, 4.5, 1.8 Hz, 2H), 3.64 (d, J=7.4 Hz, 2H), 3.33 (s, 3H), 3.24 (td, J=11.7, 2.1 Hz, 2H), 2.40-2.33 (m, 2H), 2.21-2.14 (m, 2H), 2.09-1.99 (m, 1H), 1.74-1.67 (m, 2H), 1.64-1.57 (m, 2H), 1.55-1.49 (m, 2H), 1.32-1.22 (m, 2H). ¹³C NMR (151 MHz, DMSO-d₆) δ: 156.1, 152.5, 150.8, 134.9, 127.8, 122.6, 102.5, 97.5, 66.7 (2C), 55.3, 38.8, 34.0, 30.3 (2C), 25.0, 24.9, 22.1, 21.8. HRMS (ASAP+, m/z): found 327.2189, calcd for C₁₉H₂₇N₄O [M+H]⁺, 327.2184.

### N-Methyl-6-(4-nitrophenyl)-N-((tetrahydro-2H-pyran-4-yl)methyl)-7H-pyrrolo[2,3-d]pyrimidin-4-amine (SH-01-76)

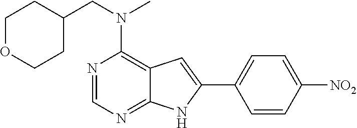 [SMILES: CN(CC1CCOCC1)c1ncnc2[nH]c(-c3ccc([N+](=O)[O-])cc3)cc12]

¹H NMR (600 MHz, DMSO-d₆) δ: 12.42 (s, 1H), 8.26 (d, J=9.0 Hz, 1H), 8.17-8.14 (m, 3H), 7.43 (s, 1H), 3.87-3.80 (m, 2H), 3.71 (d, J=7.4 Hz, 2H), 3.43 (s, 3H), 3.25 (td, J=11.7, 2.1 Hz, 2H), 2.10-2.01 (m, 1H), 1.57-1.51 (m, 2H), 1.30 (qd, J=12.6, 4.6 Hz, 2H). ¹³C NMR (150 MHz, DMSO-d₆) δ: 156.7, 153.6, 152.3, 145.6, 138.0, 130.6, 125.1 (2C), 124.2 (2C), 103.6, 103.3, 66.7 (2C), 55.4, 38.9, 33.8, 30.3 (2C). HRMS (ASAP+, m/z): found 368.1727 calcd for C₁₉H₂₂N₅O₃ [M+H]⁺368.1723

### 6-(4-Aminophenyl)-N-methyl-N-((tetrahydro-2H-pyran-4-yl)methyl)-7H-pyrrolo[2,3-d]pyrimidin-4-amine (SH-01-82)

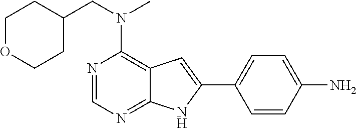 [SMILES: CN(CC1CCOCC1)c1ncnc2[nH]c(-c3ccc(N)cc3)cc12]

¹H NMR (600 MHz, DMSO-d₆) δ: 11.78 (s, 1H), 8.04 (s, 1H), 7.53 (d, J=8.6 Hz, 2H), 6.73 (s, 1H), 6.59 (d, J=8.6 Hz, 2H), 5.26 (s, 2H), 3.86-3.80 (m, 2H), 3.66 (d, J=7.4 Hz, 2H), 3.36 (s, 3Hf), 3.24 (td, J=11.7, 2.1 Hz, 2H), 2.10-2.01 (m, 1H), 1.56-1.51 (m, 2H), 1.29 (qd, J=12.5, 4.4 Hz, 2H). ¹³C NMR (150 MHz, DMSO-d₆) δ: 155.9, 152.4, 150.1, 148.3, 134.5, 125.8 (2C), 119.3, 113.9 (2C), 103.4, 95.3, 66.8 (2C), 55.3, 38.8, 34.2, 30.3 (2C). HRMS (ASAP+, m/z): found 338.1984 calcd. for C₁₉H₂₄N₅O [M+H]⁺ 338.1981.

### N-Methyl-6-(pyridin-4-yl)-N-((tetrahydro-2H-pyran-4-yl)methyl)-7H-pyrrolo[2,3-d]pyrimidin-4-amine (SH-01-92)

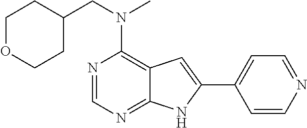 [SMILES: CN(CC1CCOCC1)c1ncnc2[nH]c(-c3ccncc3)cc12]

¹H NMR (600 MHz, DMSO-d₆) δ: 12.37 (s, 1H), 8.57 (s, 2H), 8.15 (s, 1H), 7.86 (d, J=5.4 Hz, 2H), 7.41 (s, 1H), 3.86-3.80 (m, 2H), 3.71 (d, J=7.4 Hz, 2H), 3.42 (s, 3H), 3.25 (td, J=11.7, 2.1 Hz, 2H), 2.11-2.04 (m, 1H), 1.56-1.50 (m, 2H), 1.30 (qd, J=12.1, 4.4 Hz, 2H). ¹³C-NMR (150 MHz, DMSO-d₆) δ: 156.7, 153.3, 152.1, 150.1 (2C), 138.5, 130.0, 118.6 (2C), 103.3, 102.4, 66.7 (2C), 55.3, 38.9, 33.8, 30.3 (2C). HRMS (ASAP+, m/z): found 324.1826 calcd for C₁₈H₂₂N₅O [M+H]⁺324.1824

### 3-(4-(4-(Methyl((tetrahydro-2H-pyran-4-yl)methyl)amino)-7H-pyrrolo[2,3-d]pyrimidin-6-yl)phenyl)oxetan-3-ol (TIA06-003)

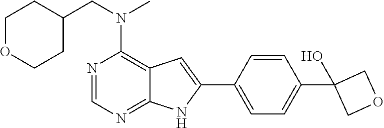 [SMILES: CN(CC1CCOCC1)c1ncnc2[nH]c(-c3ccc(C4(O)COC4)cc3)cc12]

¹H NMR (600 MHz, DMSO-d₆) δ 12.14 (s, 1H), 8.11 (s, 1H), 7.91 (d, J=8.6 Hz, 2H), 7.63 (d, J=8.6 Hz, 2H), 7.08 (s, 1H), 6.37 (s, 1H), 4.78 (d, J=6.7 Hz, 2H), 4.71 (d, J=6.8 Hz, 2H), 3.87-3.79 (m, 2H), 3.69 (d, J=7.5 Hz, 2H), 3.40 (s, 3H), 3.30-3.19 (m, 2H), 2.12-1.97 (m, 1H), 1.58-1.49 (m, 2H), 1.37-1.20 (m, 2H); ¹³C NMR (150 MHz, DMSO-d₆) δ 156.4, 152.8, 151.1, 143.0, 132.7, 130.4, 125.0 (2C), 124.5 (2C), 103.3, 98.9, 85.1 (2C), 73.9, 66.7 (2C), 55.3, 38.9, 33.9, 30.3 (2C), signal at 38.9 in DMSO peak—found from HSQC and HMBC; IR (neat, cm-1): 3343 (br, m), 2847 (m), 1568 (s), 1410 (s), 971 (s), 847 (s); HRMS (ES+, m/z): found 395.2085, calcd. C₂₂H₂₇N₄O₃, [M+H]⁺, 395.2083.

### 6-(3,6-Dihydro-2H-pyran-4-yl)-N-methyl-N-((tetrahydro-2H-pyran-4-yl)methyl)-7H-pyrrolo[2,3-d]pyrimidin-4-amine (FAB 04-09)

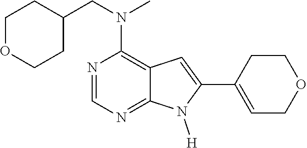 [SMILES: CN(CC1CCOCC1)c1ncnc2[nH]c(C3=CCOCC3)cc12]

¹H NMR (400 MHz, DMSO-d₆) δ 11.78 (s, 1H), 8.08 (s, 1H), 6.53 (d, J=2.2 Hz, 1H), 6.37 (t, J=3.0 Hz, 1H), 4.23 (q, J=2.7 Hz, 2H), 3.87-3.77 (m, 4H), 3.64 (d, J=7.3 Hz, 2H), 3.34 (s, 3H), 3.24 (td, J=11.7, 2.0 Hz, 2H), 2.46-2.42 (m, 2H), 2.10-1.97 (m, 1H), 1.55-1.48 (m, 2H), 1.34-1.19 (m, 2H); 13C NMR (101 MHz, DMSO-d₆) δ 156.3, 152.7, 151.2, 133.4, 125.6, 120.8, 102.5, 98.4, 66.7 (2C), 64.7, 63.3, 55.3, 39.1, 34.0, 30.3 (2C), 25.1; IR (neat, cm-1): 3189 (w), 2936 (w), 2838 (w), 1667 (s), 1582 (s), 1506 (s), 1348 (s), 941 (s), 849 (s), 764 (s); HRMS (ASAP+, m/z): found 329.1982, calcd for C₁₃H₂₅N₄O₂, [M+H]⁺, 329.1978.

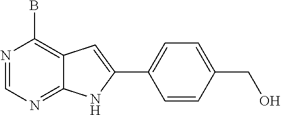

### (4-(4-(Methyl(2-methylbenzyl)amino)-7H-pyrrolo[2,3-d]pyrimidin-6-yl)phenyl)methanol (TIA02-072)

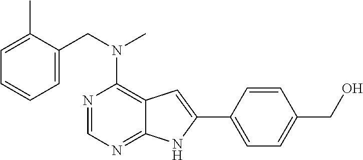 [SMILES: Cc1ccccc1CN(C)c1ncnc2[nH]c(-c3ccc(CO)cc3)cc12]

¹H NMR (600 MHz, DMSO-d₆) δ: 12.14 (s, 1H), 8.12 (s, 1H), 7.80-7.76 (m, 2H), 7.37-7.32 (m, 2H), 7.24-7.20 (m, 1H), 7.18-7.13 (m, 1H), 7.13-7.09 (m, 1H), 7.01-6.97 (m, 1H), 6.95 (s, 1H), 5.19 (t, J=5.7 Hz, 1H), 5.00 (s, 2H), 4.50 (d, J=5.7 Hz, 2H), 3.38 (s, 3H), 2.31 (s, 3H); ¹³C NMR (151 MHz, DMSO-d₆) δ: 156.5, 152.9, 151.1, 141.7, 135.9, 135.6, 133.3, 130.2, 130.0, 126.8 (2C), 126.6, 125.9, 125.8, 124.5 (2C), 103.3, 98.4, 62.6, 51.1, 37.5, 18.7; HRMS (ASAP+, m/z): found 359.1867, calcd for C₂₂H₂₃N₄O, [M+H]⁺, 359.1872.

### (4-(4-(Methyl(4-methylbenzyl)amino)-7H-pyrrolo[2,3-d]pyrimidin-6-yl)phenyl)methanol (TIA02-074)

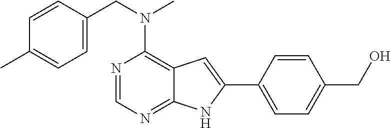 [SMILES: Cc1ccc(CN(C)c2ncnc3[nH]c(-c4ccc(CO)cc4)cc23)cc1]

¹H NMR (600 MHz, DMSO-d₆) δ: 12.14 (s, 1H), 8.14 (s, 1H), 7.82-7.78 (m, 2H), 7.36-7.32 (m, 2H), 7.19-7.15 (m, 2H), 7.14-7.10 (m, 2H), 7.02 (s, 1H), 5.19 (t, J=5.7 Hz, 1H), 5.00 (s, 2H), 4.50 (d, J=5.7 Hz, 2H), 3.33 (s, 3H), 2.26 (s, 3H); ¹³C NMR (151 MHz, DMSO-d₆) δ: 156.4, 152.9, 151.1, 141.7, 135.9, 135.3, 133.3, 130.0, 129.1 (2C), 127.0 (2C), 126.8 (2C), 124.5 (2C), 103.2, 98.5, 62.6, 52.4, 37.2, 20.6; HRMS (ASAP+, m/z): found 359.1870, calcd for C₂₂H₂₃N₄O, [M+H]⁺, 359.1872.

### (4-(4-(Methyl((6-methylpyridin-2-yl)methyl)amino)-7H-pyrrolo[2,3-d]pyrimidin-6-yl)phenyl)methanol (TIA03-096)

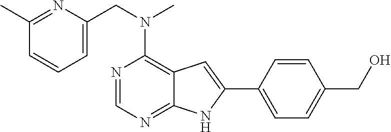 [SMILES: Cc1cccc(CN(C)c2ncnc3[nH]c(-c4ccc(CO)cc4)cc23)n1]

¹H NMR (400 MHz, DMSO-d₆) δ: 12.14 (s, 1H), 8.11 (s, 1H), 7.81-7.77 (m, 2H), 7.62-7.57 (m, 1H), 7.37-7.33 (m, 2H), 7.13-7.10 (m, 1H), 7.04 (s, 1H), 6.98-6.94 (m, 1H), 5.19 (t, J=5.7 Hz, 1H), 5.05 (s, 2H), 4.51 (d, J=5.7 Hz, 2H), 3.47 (s, 3H), 2.47 (s, 3H); ¹³C NMR (101 MHz, DMSO-d₆) δ: 157.7, 157.5, 156.3, 152.9, 151.0, 141.7, 137.1, 133.3, 129.9, 126.9 (2C), 124.5 (2C), 121.4, 117.5, 103.4, 98.5, 62.6, 55.2, 38.2, 24.1; HRMS (ASAP+, m/z): found 360.1825, calcd for C₂₁H₂₂N₅O, [M+H]⁺, 360.1824.

### (4-(4-(Methyl(pyridin-2-ylmethyl)amino)-7H-pyrrolo[2,3-d]pyrimidin-6-yl)phenyl)methanol (TIA02-076)

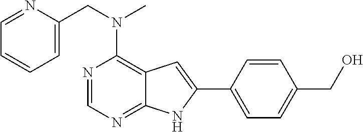 [SMILES: CN(Cc1ccccn1)c1ncnc2[nH]c(-c3ccc(CO)cc3)cc12]

¹H NMR (600 MHz, DMSO-d₆) δ: 12.14 (s, 1H), 8.56-8.51 (m, 1H), 8.11 (s, 1H), 7.82-7.77 (m, 2H), 7.75-7.69 (m, 1H), 7.37-7.33 (m, 2H), 7.28-7.24 (m, 1H), 7.24-7.20 (m, 1H), 7.03 (s, 1H), 5.20 (t, J=5.7 Hz, 1H), 5.11 (s, 2H), 4.51 (d, J=5.7 Hz, 2H), 3.48 (s, 3H); ¹³C NMR (151 MHz, DMSO-d₆) δ: 158.3, 156.4, 152.9, 151.0, 149.2, 141.7, 136.8, 133.3, 129.9, 126.8 (2C), 124.5 (2C), 122.1, 120.9, 103.4, 98.5, 62.6, 55.0, 38.2; HRMS (ASAP+, m/z): found 346.1663, calcd for C₂₀H₂₀N₅O, [M+H]⁺, 346.1668.

### (4-(4-(Methyl(pyridin-3-ylmethyl)amino)-7H-pyrrolo[2,3-d]pyrimidin-6-yl)phenyl)methanol (TIA086)

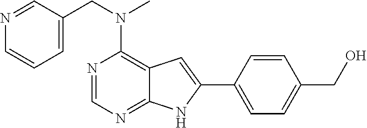 [SMILES: CN(Cc1cccnc1)c1ncnc2[nH]c(-c3ccc(CO)cc3)cc12]

¹H NMR (400 MHz, DMSO-d₆) δ: 12.17 (s, 1H), 8.57-8.52 (m, 1H), 8.49-8.43 (m, 1H), 8.15 (s, 1H), 7.85-7.79 (m, 2H), 7.71-7.64 (m, 1H), 7.38-7.30 (m, 3H), 7.10-7.05 (m, 1H), 5.19 (t, J=5.7 Hz, 1H), 5.06 (s, 2H), 4.51 (d, J=5.7 Hz, 2H), 3.41 (s, 3H); ¹³C NMR (101 MHz, DMSO-d₆) δ: 156.3, 152.9, 151.0, 148.8, 148.2, 141.7, 134.9, 134.1, 133.5, 129.9, 126.8 (2C), 124.5 (2C), 123.6, 103.4, 98.4, 62.6, 50.4, 37.5; HRMS (ASAP+, m/z): found 346.1673, calcd for C₂₀H₂₀N₅O, [M+H]⁺, 346.1668.

### (4-(4-(Methyl(pyridin-4-ylmethyl)amino)-7H-pyrrolo[2,3-d]pyrimidin-6-yl)phenyl)methanol (TIA02-052)

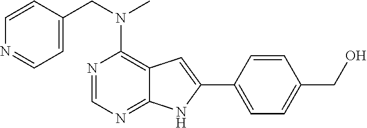 [SMILES: CN(Cc1ccncc1)c1ncnc2[nH]c(-c3ccc(CO)cc3)cc12]

¹H NMR (400 MHz, DMSO-d₆) δ: 12.18 (s, 1H), 8.52-8.46 (m, 2H), 8.12 (s, 1H), 7.85-7.78 (m, 2H), 7.38-7.32 (m, 2H), 7.28-7.22 (m, 2H), 7.05 (s, 1H), 5.20 (t, J=5.7 Hz, 1H), 5.06 (s, 2H), 4.51 (d, J=5.7 Hz, 2H), 3.44 (s, 3H); ¹³C NMR (101 MHz, DMSO-d₆) δ: 156.3, 152.9, 151.0, 149.7 (2C), 147.9, 141.8, 133.6, 129.9, 126.8 (2C), 124.5 (2C), 122.0 (2C), 103.3, 98.4, 62.6, 52.1, 37.9; HRMS (ASAP+, m/z): found 346.1662, calcd for C₂₀H₂₀N₅O, [M+H]⁺, 346.1668.

### 2-(((6-(4-(Hydroxymethyl)phenyl)-7H-pyrrolo[2,3-d]pyrimidin-4-yl)(methyl)amino)methyl)phenol (TIA03-126)

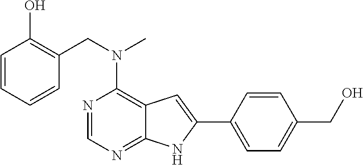 [SMILES: CN(Cc1ccccc1O)c1ncnc2[nH]c(-c3ccc(CO)cc3)cc12]

¹H NMR (400 MHz, DMSO-d₆) δ: 12.18 (s, 1H), 10.36 (s, 1H), 8.15 (s, 1H), 7.82-7.75 (m, 2H), 7.38-7.31 (m, 2H), 7.14-7.06 (m, 2H), 7.01 (s, 1H), 6.88-6.81 (m, 1H), 6.78-6.69 (m, 1H), 5.19 (t, J=5.7 Hz, 1H), 4.87 (s, 2H), 4.51 (d, J=5.6 Hz, 2H), 3.43 (s, 3H); ¹³C NMR (151 MHz, DMSO-d₆) δ: 156.1, 155.5, 152.6, 150.7, 141.8, 133.4, 129.9, 128.5, 128.3, 126.9 (2C), 124.5 (2C), 123.9, 119.0, 115.6, 103.4, 98.6, 62.6, 49.1, 37.6; HRMS (ES+, m/z): found 361.1663, calcd for C₂₁H₂₁N₄O₂, [M+H]⁺, 361.1665.

### 2-(((6-(4-(Hydroxymethyl)phenyl)-7H-pyrrolo[2,3-d]pyrimidin-4-yl)(methyl)amino)methyl)-4-methylphenol (TIA04-139)

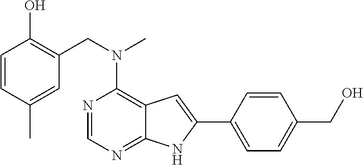 [SMILES: Cc1ccc(O)c(CN(C)c2ncnc3[nH]c(-c4ccc(CO)cc4)cc23)c1]

¹H NMR (400 MHz, DMSO-d₆) δ: 12.19 (s, 1H), 10.13 (s, 1H), 8.15 (s, 1H), 7.82-7.76 (m, 2H), 7.38-7.32 (m, 2H), 7.04-6.99 (m, 1H), 6.93-6.86 (m, 2H), 6.77-6.70 (m, 1H), 5.20 (t, J=5.7 Hz, 1H), 4.83 (s, 2H), 4.51 (d, J=5.7 Hz, 2H), 3.43 (s, 3H), 2.14 (s, 3H); ¹³C NMR (101 MHz, DMSO-d₆) δ: 156.1, 153.1, 152.6, 150.7, 141.7, 133.4, 129.9, 128.7 (2C), 127.3, 126.8 (2C), 124.5 (2C), 123.6, 115.5, 103.3, 98.6, 62.6, 49.0, 37.6, 20.3; HRMS (ASAP+, m/z): found 375.1814, calcd for C₂₂H₂₃N₄O₂, [M+H]⁺, 375.1821.

### (4-(4-((2-Fluorobenzyl)(methyl)amino)-7H-pyrrolo[2,3-d]pyrimidin-6-yl)phenyl)methanol (TIA05-086)

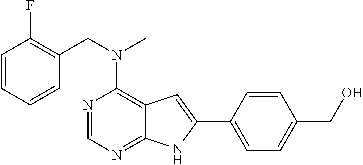 [SMILES: CN(Cc1ccccc1F)c1ncnc2[nH]c(-c3ccc(CO)cc3)cc12]

¹H NMR (600 MHz, DMSO-d₆) δ: 12.16 (s, 1H), 8.13 (s, 1H), 7.84-7.79 (m, 2H), 7.37-7.34 (m, 2H), 7.34-7.28 (m, 1H), 7.26-7.17 (m, 2H), 7.16-7.10 (m, 1H), 7.06 (s, 1H), 5.19 (t, J=5.7 Hz, 1H), 5.08 (s, 2H), 4.51 (d, J=5.8 Hz, 2H), 3.42 (s, 3H); ¹³C NMR (151 MHz, DMSO-d₆) δ: 160.4 (d, ¹J_{CF}=244 Hz, 1C), 156.3, 152.9, 151.0, 141.7, 133.5, 129.9, 128.85 (d, ³J_{CF}=8.0 Hz, 1C), 128.76 (d, ³J_{CF}=4.5 Hz, 1C), 126.8 (2C), 125.1 (d, ²J_{CF}=14.7 Hz, 1C), 124.51 (2C), 124.49, 115.3 (d, ²J_{CF}=21.2 Hz, 1C), 103.4, 98.3, 62.6, 46.9, 37.7; HRMS (ES+, m/z): found 363.1622, calcd for C₂₁H₂₀N₄OF, [M+H]⁺, 363.1621.

### (4-(4-(Benzyl(ethyl)amino)-7H-pyrrolo[2,3-d]pyrimidin-6-yl)phenyl)methanol (TIA084)

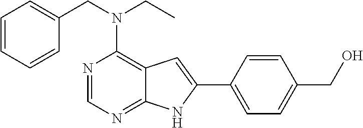 [SMILES: CCN(Cc1ccccc1)c1ncnc2[nH]c(-c3ccc(CO)cc3)cc12]

¹H NMR (600 MHz, DMSO-d₆) δ: 12.12 (s, 1H), 8.14 (s, 1H), 7.79-7.75 (m, 2H), 7.36-7.28 (m, 6H), 7.27-7.21 (m, 1H), 6.84 (s, 1H), 5.19 (t, J=5.7 Hz, 1H), 5.03 (s, 2H), 4.50 (d, J=5.6 Hz, 2H), 3.78 (q, J=7.0 Hz, 2H), 1.24 (t, J=7.0 Hz, 3H); ¹³C NMR (151 MHz, DMSO-d₆) δ: 155.7, 152.9, 151.1, 141.8, 138.9, 133.5, 129.9, 128.4 (2C), 127.0 (2C), 126.84 (2C), 126.82, 124.5 (2C), 102.5, 98.1, 62.6, 50.5, 43.1, 13.3; HRMS (ASAP+, m/z): found 359.1866, calcd for C₂₂H₂₃N₄O, [M+H]⁺, 359.1872.

### (4-(4-(Benzyl(methyl-d₃)amino)-7H-pyrrolo[2,3-d]pyrimidin-6-yl)phenyl)methanol (TIA05-046)

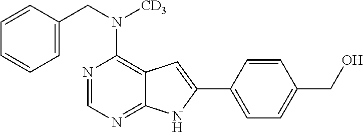 [SMILES: [2H]C([2H])([2H])N(Cc1ccccc1)c1ncnc2[nH]c(-c3ccc(CO)cc3)cc12]

¹H NMR (600 MHz, DMSO-d₆) δ: 12.15 (s, 1H), 8.14 (s, 1H), 7.83-7.78 (m, 2H), 7.37-7.31 (m, 4H), 7.29-7.26 (m, 2H), 7.26-7.22 (m, 1H), 7.03 (s, 1H), 5.19 (t, J=5.7 Hz, 1H), 5.04 (s, 2H), 4.51 (d, J=5.7 Hz, 2H); ¹³C NMR (151 MHz, DMSO-d₆) δ: 156.4, 152.9, 151.1, 141.7, 138.5, 133.3, 130.0, 128.5 (2C), 127.0 (2C), 126.9, 126.8 (2C), 124.5 (2C), 103.2, 98.5, 62.6, 52.6, 36.6; HRMS (ES+, m/z): found 348.1902, calcd for C₂₁H₁₈D₃N₄O, [M+H]⁺, 348.1904.

### (R)-(4-(4-(Methyl(1-phenylethyl)amino)-7H-pyrrolo[2,3-d]pyrimidin-6-yl)phenyl)methanol (TIA05-088)

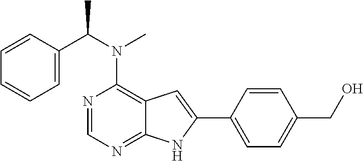 [SMILES: C[C@H](c1ccccc1)N(C)c1ncnc2[nH]c(-c3ccc(CO)cc3)cc12]

¹H NMR (600 MHz, DMSO-d₆) δ: 12.15 (s, 1H), 8.17 (s, 1H), 7.86-7.81 (m, 2H), 7.39-7.31 (m, 6H), 7.29-7.24 (m, 1H), 7.08 (s, 1H), 6.45 (s, 1H), 5.19 (t, J=5.7 Hz, 1H), 4.51 (d, J=5.7 Hz, 2H), 3.07 (s, 3H), 1.60 (d, J=7.0 Hz, 3H); ¹³C NMR (151 MHz, DMSO-d₆) δ: 156.5, 153.0, 151.0, 141.7, 141.6, 133.2, 130.0, 128.4 (2C), 126.90, 126.86 (2C), 126.8 (2C), 124.5 (2C), 103.5, 98.7, 62.6, 52.1, 31.6, 16.2; HRMS (ES+, m/z): found 359.1874, calcd for C₂₂H₂₃N₄O, [M+H]⁺, 359.1872.

### (R)-(4-(4-((1-Phenylethyl)amino)-7H-pyrrolo[2,3-d]pyrimidin-6-yl)phenyl)methanol (JH06-118)

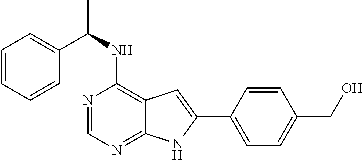 [SMILES: C[C@@H](Nc1ncnc2[nH]c(-c3ccc(CO)cc3)cc12)c1ccccc1]

Experimental details are reported in Kaspersen et al. Eur. J. Pharm. Sci. (2014), 59, 69-82.

### (S)-2-((6-(4-(Hydroxymethyl)phenyl)-7H-pyrrolo[2,3-d]pyrimidin-4-yl)(methyl)amino)-2-phenylethan-1-ol (TIA05-008)

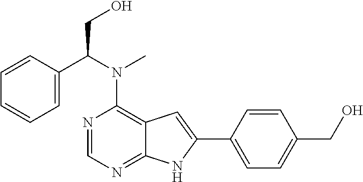 [SMILES: CN(c1ncnc2[nH]c(-c3ccc(CO)cc3)cc12)[C@H](CO)c1ccccc1]

¹H NMR (600 MHz, DMSO-d₆) δ: 12.12 (s, 1H), 8.14 (s, 1H), 7.84-7.80 (m, 2H), 7.39-7.31 (m, 6H), 7.29-7.23 (m, 1H), 7.07 (s, 1H), 6.29 (s, 1H), 5.19 (t, J=5.7 Hz, 1H), 4.99 (t, J=5.3 Hz, 1H), 4.51 (d, J=5.6 Hz, 2H), 4.12-4.05 (m, 1H), 4.02-3.95 (m, 1H), 3.21 (s, 3H); ¹³C NMR (151 MHz, DMSO-d₆) δ: 157.3, 153.0, 150.9, 141.6, 139.1, 133.1, 130.0, 128.4 (2C), 127.4 (2C), 127.0, 126.8 (2C), 124.5 (2C), 103.6, 98.8, 62.6, 60.6, 58.9, 32.4; HRMS (ASAP+, m/z): found 375.1824, calcd for C₂₂H₂₃N₄O₂, [M+H]⁺, 375.1821.

### (4-(4-((Cyclohexylmethyl)(methyl)amino)-7H-pyrrolo[2,3-d]pyrimidin-6-yl)phenyl)methanol (TIA05-090)

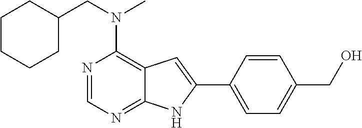 [SMILES: CN(CC1CCCCC1)c1ncnc2[nH]c(-c3ccc(CO)cc3)cc12]

¹H NMR (600 MHz, DMSO-d₆) δ: 12.06 (s, 1H), 8.09 (s, 1H), 7.84-7.80 (m, 2H), 7.38-7.33 (m, 2H), 7.02 (s, 1H), 5.18 (t, J=5.7 Hz, 1H), 4.51 (d, J=6.3 Hz, 2H), 3.64 (d, J=7.4 Hz, 2H), 3.38 (s, 3H), 1.85-1.77 (m, 1H), 1.71-1.63 (m, 4H), 1.63-1.47 (m, 1H), 1.23-1.10 (m, 3H), 1.05-0.96 (m, 2H); ¹³C NMR (151 MHz, DMSO-d₆) δ: 156.4, 152.8, 151.0, 141.6, 132.9, 130.1, 126.8 (2C), 124.4 (2C), 103.2, 98.5, 62.6, 55.8, 40.1, 38.9, 36.5, 30.2 (2C), 26.1, 25.4 (2C); HRMS (ES+, m/z): found 351.2184, calcd for C₂₁H₂₇N₄O, [M+H]⁺, 351.2185.

### Further Suitable Compounds:

### N-(Cyclohexylmethyl)-6-(3,6-dihydro-2H-pyran-4-yl)-N-methyl-7H-pyrrolo[2,3-d]pyrimidin-4-amine (HHMT-170)

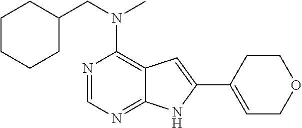 [SMILES: CN(CC1CCCCC1)c1ncnc2[nH]c(C3=CCOCC3)cc12]

¹H NMR (600 MHz, CDCl₃) δ: 10.85 (br s, 1H), 8.24 (s, 1H), 6.43 (s, 1H), 6.17 (s, 1H), 4.40 (br s, 2H), 3.96 (t, 2H, J=5.4 Hz), 3.63 (d, 2H, J=6.0 Hz), 3.38 (s, 3H), 2.55 (br s, 2H), 1.87-1.84 (m, 1H), 1.74 (br d, 4H, J=11.4 Hz), 1.67 (br d, 1H, J=10.2 Hz), 1.25-1.17 (m, 3H), 1.06-1.00 (m, 2H); ¹³C NMR (150 MHz, CDCl₃) δ: 157.0, 152.5, 151.5, 133.4, 126.3, 120.4, 103.3, 98.7, 65.6, 64.1, 57.1, 39.2, 37.3, 30.9 (2C), 26.5, 25.9 (2C), 25.8; HRMS (ASAP+, m/z): found 327.2180 (calcd for C₁₉H₂₇N₄O, [M+H]⁺, 327.2185; mp. 202-207° C.

### 6-(Cyclohex-1-en-1-yl)-N-(cyclohexylmethyl)-N-methyl-7Hpyrrolo[2,3-d]pyrimidin-4-amine (HHMT-070)

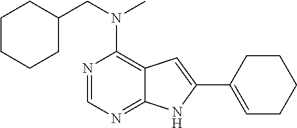 [SMILES: CN(CC1CCCCC1)c1ncnc2[nH]c(C3=CCCCC3)cc12]

¹H NMR (600 MHz, CD₃OD:CDCl₃, 1:1) δ: 8.08 (s, 1H), 6.39 (s, 1H), 6.27 (br s, 1H), 3.61 (d, 2H, J=7.8 Hz), 3.35 (s, 3H), 2.42 (br s, 2H), 2.27 (br s, 2H), 1.88-1.70 (m, 10H), 1.27-1.19 (m, 3H), 1.07-1.01 (m, 2H); ¹³C NMR (100 MHz, CD₃OD:CDCl₃, 1:1) δ: 155.9, 150.1, 149.5, 134.9, 127.1, 122.6, 102.6, 96.9, 56.5, 38.5, 36.4, 29.9 (2C), 25.5, 25.0 (2C), 24.7, 24.6, 21.6, 21.2; HRMS (ASAP+, m/z): found 325.2388, calcd for C₂₀H₂₉N₄, [M+H]⁺, 325.2392; mp. 234-237° C.

### 3-(4-(4-((3-Methoxybenzyl)(methyl)amino)-7H-pyrrolo[2,3-d]pyrimidin-6-yl)phenyl)propanoic acid (FAB 01-23)

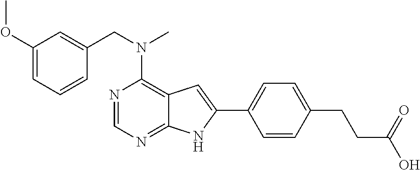 [SMILES: COc1cccc(CN(C)c2ncnc3[nH]c(-c4ccc(CCC(=O)O)cc4)cc23)c1]

¹H NMR (400 MHz, DMSO-d₆) δ: 12.14 (s, 1H), 8.15 (s, 1H), 7.76 (d, J=8.0 Hz, 2H), 7.30-7.20 (m, 3H), 7.00 (s, 1H), 6.87-6.79 (m, 3H), 5.02 (s, 2H), 3.71 (s, 3H), 3.37 (s, 3H), 2.83 (t, J=7.5 Hz, 2H), 2.56 (d, J=7.6 Hz, 2H). 3-(4-(4-((3,4-Dimethoxybenzyl)(methyl)amino)-7H-pyrrolo[2,3-d]pyrimidin-6-yl)phenyl)propanoic acid (FAB 01-24)

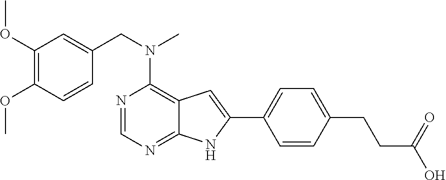 [SMILES: COc1ccc(CN(C)c2ncnc3[nH]c(-c4ccc(CCC(=O)O)cc4)cc23)cc1OC]

¹H NMR (400 MHz, DMSO-d₆) δ: 12.13 (s, 1H), 8.16 (s, 1H), 7.76 (d, J=8.1 Hz, 2H), 7.27 (d, J=8.1 Hz, 2H), 7.00 (s, 1H), 6.95 (d, J=2.14 Hz, 1H), 6.89 (d, J=8.32 Hz, 1H), 6.77 (dd, J=8.2, 2.0 Hz, 1H), 4.96 (s, 2H), 3.71 (s, 3H), 3.7 (s, 3H), 3.34 (s, 3H), 2.83 (t, J=7.6 Hz, 2H), 2.54 (d, J=7.5 Hz, 2H).

### (4-(4-(2-Phenylpyrrolidin-1-yl)-7H-pyrrolo[2,3-d]pyrimidin-6-yl)phenyl)methanol (TIA05-178)

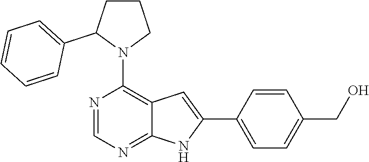 [SMILES: OCc1ccc(-c2cc3c(N4CCCC4c4ccccc4)ncnc3[nH]2)cc1]

¹H NMR (600 MHz, DMSO-d₆) δ: 12.04 (s, 1H), 8.03 (br s, 1H), 7.90-7.53 (m, 3H), 7.36-7.32 (m, 2H), 7.32-7.26 (m, 2H), 7.26-7.23 (m, 2H), 7.21-7.15 (m, 1H), 5.65-5.60 (m, 1H), 5.19 (t, J=5.7 Hz, 1H), 4.50 (d, J=5.7 Hz, 2H), 4.23 (br s, 1H), 3.95 (br s, 1H), 2.41-2.38 (m, 1H), 2.02 (br s, 1H), 1.95-1.85 (m, 2H); ¹³C NMR (151 MHz, DMSO-d₆) δ: 154.3, 152.4, 151.3, 144.3, 141.6, 133.1, 130.0, 128.2 (2C), 126.9 (2C), 126.3, 125.5 (2C), 124.3 (2C), 103.8, 98.1, 62.6, 61.1, 48.8, 34.4, 22.5; HRMS (ES+, m/z): found 371.1877, calcd for C₂₃H₂₃N₄O, [M+H]⁺, 371.1872.

### 6-(4-Methoxyphenyl)-4-(2-methylpyrrolidin-1-yl)-7H-pyrrolo[2,3-d]pyrimidine (MS1-60-134)

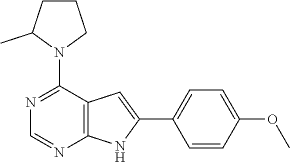

¹H NMR (400 MHz, DMSO-d₆) δ: 12.08 (s, 1H), 8.08 (s, 1H), 7.83-7.81 (m, 2H), 7.34-7.32 (m, 2H), 7.00 (br s, 1H), 5.35 (br s, 1H), 4.54-4.53 (m, 1H), 4.49 (s, 2H), 3.91 (m, 1H), 3.70 (m, 1H), 2.12-1.96 (m, 3H), 1.71-1.69 (m, 1H), 1.24-1.23 (m, 3H); ¹³C NMR (100 MHz, DMSO-d₆) δ: 154.1, 152.4, 151.4, 141.9, 133.6, 130.3, 127.2 (2C), 124.75 (2C), 103.9, 98.4, 62.9, 53.6, 48.3, 32.1, 23.0, 19.9; HRMS (ASAP+, m/z): found 309.1716, calcd for C₁₈H₂₁N₄O, [M+H]⁺, 309.1715; mp. 224-226° C.

### 6-(4-Methoxyphenyl)-4-(pyrrolidin-1-yl)-7H-pyrrolo[2,3-d]pyrimidine (MS-46-93)

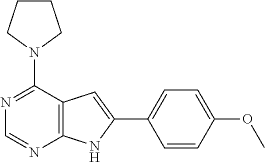 [SMILES: COc1ccc(-c2cc3c(N4CCCC4)ncnc3[nH]2)cc1]

¹H NMR (400 MHz, DMSO-d₆) δ: 12.03 (s, 1H), 8.09 (s, 1H), 7.84-7.82 (m, 2H), 7.36-7.34 (m, 2H), 7.05 (br s, 1H), 5.23-5.20 (m, 1H), 4.51-4.50 (m, 2H), 3.77 (br s, 4H), 1.98 (br s, 4H); ¹³C NMR (100 MHz, DMSO-d₆) δ: 154.5, 152.2, 151.5, 141.5, 133.0, 130.2, 126.9 (2C), 124.4 (2C), 103.9, 98.0, 62.6, 47.6 (2C), 25.0 (2C); HRMS (ASAP+, m/z): found 295.1553, calcd for C₁₇H₁₉N₄O, [M+H]⁺, 295.1559.

### 6-Phenyl-4-(2-(tetrahydro-2H-pyran-4-yl)pyrrolidin-1-yl)-7H-pyrrolo[2,3-d]pyrimidine (SH-01-112)

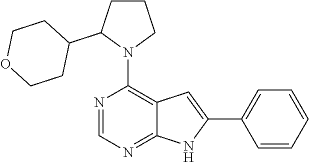 [SMILES: c1ccc(-c2cc3c(N4CCCC4C4CCOCC4)ncnc3[nH]2)cc1]

¹H NMR (600 MHz, DMSO-d₆) δ: 12.09 (s, 1H), 8.12 (s, 1H), 7.88 (d, J=7.1 Hz, 2H), 7.41 (t, J=7.8 Hz, 2H), 7.28 (t, J=7.4 Hz, 1H), 7.05 (s, 1H), 4.53-4.47 (m, 1H), 4.01-3.71 (m, 4H), 3.27-3.09 (m, 2H), 2.22-2.17 (m, 1H), 2.07-1.97 (m, 2H), 1.94-1.78 (m, 2H), 1.50-1.32 (m, 4H). ¹³C NMR (150 MHz, DMSO-d₆) δ:155.1, 152.5, 151.3, 133.0, 131.6, 128.8 (2C), 127.2, 124.6 (2C), 104.1, 98.5, 67.2 (2C), 61.1, 48.9, 37.4, 27.7 (2C), 25.6, 23.7. HRMS (ASAP+, m/z): found 349.2034 calcd for C₂₁H₂₆N₅O₂ [M+H]⁺ 349.2028.

### 4-(4-(4-(2-(Tetrahydro-2H-pyran-4-yl)pyrrolidin-1-yl)-7H-pyrrolo[2,3-d]pyrimidin-6-yl)benzyl)morpholine (SH-01-118)

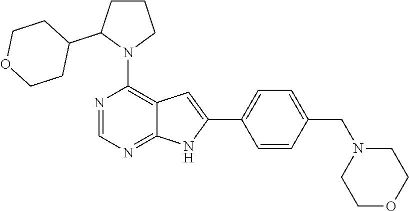 [SMILES: c1nc(N2CCCC2C2CCOCC2)c2cc(-c3ccc(CN4CCOCC4)cc3)[nH]c2n1]

¹H NMR (600 MHz, DMSO-d₆) δ: 12.07 (s, 1H), 8.11 (s, 1H), 7.83 (d, J=8.3 Hz, 2H), 7.34 (d, J=8.2 Hz, 2H), 7.01 (s, 1H), 4.53-4.47 (m, 1H), 3.97-3.74 (m, 4H), 3.60-3.56 (m, 4H), 3.47 (s, 2H), 3.25-3.11 (m, 2H), 2.39-2.34 (m, 4H), 2.21 (s, 1H), 2.07-1.97 (m, 2H), 1.95-1.79 (m, 2H), 1.50-1.32 (m, 4H). ¹³C NMR (150 MHz, DMSO-d₆) δ: 155.1, 152.5, 151.2, 136.8, 133.0, 130.4, 129.4 (2C), 124.5 (2C), 104.0, 98.3, 67.3 (2C), 66.2 (2C), 62.1, 61.1, 53.2 (2C), 48.9, 37.5, 27.7 (2C), 25.6, 23.8. HRMS (ASAP+, m/z): found 448.2719 calcd for C₂₁H₂₆N₅O₂ [M+H]⁺ 448.2713.

### N-(2,6-Difluoro-3-methylbenzyl)-N-methyl-6-phenyl-7H-pyrrolo[2,3-d]pyrimidin-4-amine (SH-01-126)

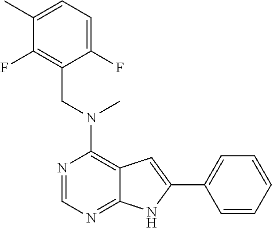 [SMILES: Cc1ccc(F)c(CN(C)c2ncnc3[nH]c(-c4ccccc4)cc23)c1F]

¹H NMR (600 MHz, DMSO-d₆) δ: 12.18 (s, 1H), 8.15 (s, 1H), 7.91-7.86 (m, 2H), 7.45-7.38 (m, 2H), 7.30-7.27 (m, 1H), 7.27-7.24 (m, 1H), 7.16 (d, J=2.2 Hz, 1H), 7.03-6.97 (m, 1H), 5.13 (s, 2H), 3.29 (s, 3H), 2.19 (s, 3H). ¹³C NMR (150 MHz, DMSO-d₆) δ: 160.3 (d, J=9.0 Hz), 158.6 (d, J=27.7 Hz), 156.2, 153.0, 150.8, 133.3, 131.5, 130.8 (dd, J=7.9, 2.5 Hz), 128.8 (2C), 127.3, 124.7 (2C), 120.4 (dd, J=21.8, 3.2 Hz), 112.7 (t, J=19.5 Hz), 110.9 (dd, J=22.0, 3.3 Hz), 103.7, 98.9, 41.3, 36.6, 13.8 (d, J=3.4 Hz). ¹⁹F NMR (565 MHz, DMSO) δ−119.3 (d, J=5.4 Hz), −120.5 (d, J=5.8 Hz). HRMS (ASAP+, m/z): found 365.1584 calcd for C₂₁H₂₆N₅O₂ [M+H]⁺ 365.1578

### N-(2,6-Difluorobenzyl)-N-methyl-6-phenyl-7H-pyrrolo[2,3-d]pyrimidin-4-amine (SH-01-127)

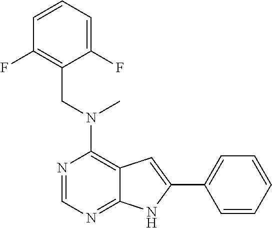 [SMILES: CN(Cc1c(F)cccc1F)c1ncnc2[nH]c(-c3ccccc3)cc12]

¹H NMR (600 MHz, DMSO-d₆) δ: 12.19 (s, 1H), 8.15 (s, 1H), 7.88 (d, J=7.1 Hz, 2H), 7.45-7.37 (m, 3H), 7.28 (t, J=7.4 Hz, 1H), 7.16 (d, J=2.1 Hz, 1H), 7.10 (t, J=8.0 Hz, 2H), 5.13 (s, 2H), 3.31 (s, 3H). ¹³C NMR (150 MHz, DMSO-d₆) δ: 161.3 (dd, J=247.7, 8.4 Hz, 2C), 156.2, 153.0, 150.8, 133.3, 131.5, 130.0 (t, J=10.4 Hz), 128.7 (2C), 127.3, 124.8 (2C), 113.4 (t, J=18.8 Hz), 111.7 (dd, J=20.7, 4.9 Hz, 2C), 103.7, 98.8, 41.4, 36.8. ¹⁹F NMR (565 MHz, DMSO) δ−116.4 (2F). HRMS (ASAP+, m/z): found 351.1428 calcd for C₂₁H₂₆N₅O₂ [M+H]⁺ 351.1421.

### N-(2,6-Difluoro-3-methylbenzyl)-N-methyl-6-(4-(morpholinomethyl)phenyl)-7H-pyrrolo[2,3-d]pyrimidin-4-amine (SH-01-128)

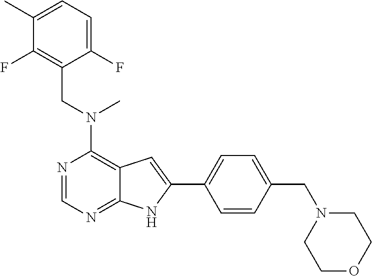 [SMILES: Cc1ccc(F)c(CN(C)c2ncnc3[nH]c(-c4ccc(CN5CCOCC5)cc4)cc23)c1F]

¹H NMR (600 MHz, DMSO-d₆) δ: 12.15 (s, 1H), 8.15 (s, 1H), 7.83 (d, J=7.9 Hz, 2H), 7.34 (d, J=7.9 Hz, 2H), 7.31-7.24 (m, 1H), 7.12 (s, 1H), 7.03-6.97 (m, 1H), 5.12 (s, 2H), 3.60-3.55 (m, 4H), 3.47 (s, 2H), 3.28 (s, 3H), 2.37-2.34 (m, 3H), 2.19 (s, 3H). ¹³C NMR (150 MHz, DMSO-d₆) δ: 160.1 (d, J=8.0 Hz), 158.6 (d, J=8.8 Hz), 156.2, 153.0, 150.8, 137.0, 133.3, 130.8 (dd, J=6.1, 2.4 Hz), 130.3, 129.3 (2C), 124.6 (2C), 120.4 (dd, J=16.6, 4.1 Hz), 112.8 (t, J=20.1 Hz), 110.9 (dd, J=23.3, 3.0 Hz), 103.7, 98.6, 66.2 (3C), 62.1, 53.2 (2C), 41.3, 36.6, 13.8 (d, J=2.5 Hz). ¹⁹F NMR (565 MHz, DMSO) δ−119.3 (d, J=5.6 Hz), −120.5 (d, J=5.6 Hz). HRMS (ASAP+, m/z): found 464.2266 calcd for C₂₁H₂₆N₅O₂ [M+H]⁺ 464.2262.

### N-(2,6-Difluorobenzyl)-N-methyl-6-(4-(morpholinomethyl)phenyl)-7H-pyrrolo[2,3-d]pyrimidin-4-amine (SH-01-129)

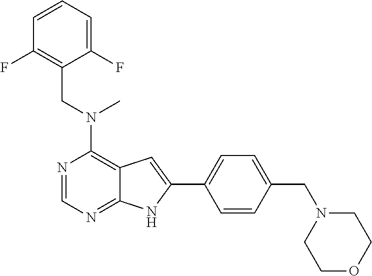 [SMILES: CN(Cc1c(F)cccc1F)c1ncnc2[nH]c(-c3ccc(CN4CCOCC4)cc3)cc12]

¹H NMR (600 MHz, DMSO-d₆) δ: 12.16 (s, 1H), 8.14 (s, 1H), 7.83 (d, J=8.3 Hz, 2H), 7.45-7.37 (m, 1H), 7.34 (d, J=8.2 Hz, 2H), 7.13 (d, J=2.2 Hz, 1H), 7.10 (t, J=8.0 Hz, 2H), 5.13 (s, 2H), 3.57 (t, J=4.6 Hz, 4H), 3.47 (s, 2H), 3.30 (s, 3H), 2.36 (t, J=4.8 Hz, 4H). ¹³C NMR (150 MHz, DMSO-d₆) δ: 161.3 (dd, J=247.7, 8.4 Hz, 2C), 156.2, 152.9, 150.7, 137.0, 133.3, 130.3, 130.0 (t, J=10.4 Hz), 129.3 (2C), 124.6 (2C), 113.4 (t, J=18.8 Hz), 111.7 (dd, J=20.7, 4.9 Hz, 2C), 103.7, 98.6, 66.2, 62.1, 53.2, 41.4, 36.8. ¹⁹F NMR (565 MHz, DMSO-d₆) δ: −116.4 (2F). HRMS (ASAP+, m/z): found 450.2108 calcd for C₂₁H₂₆N₅O₂ [M+H]⁺ 450.2105.

### N-(3-Chlorobenzyl)-N-methyl-6-(4-(morpholinomethyl)phenyl)-7H-pyrrolo[2,3-d]pyrimidin-4-amine (LR-2-88)

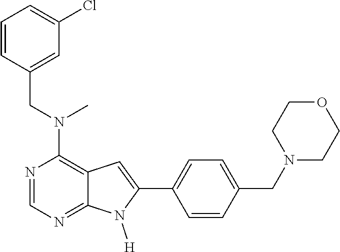 [SMILES: CN(Cc1cccc(Cl)c1)c1ncnc2[nH]c(-c3ccc(CN4CCOCC4)cc3)cc12]

¹H NMR (600 MHz, DMSO-d₆) δ: 12.19 (s, 1H), 8.15 (s, 1H), 7.81 (d, J=8.3 Hz, 2H), 7.37-7.31 (m, 5H), 7.25-7.23 (m, 1H), 7.05 (s, 1H), 5.04 (s, 2H), 3.57 (t, J=4.7 Hz, 4H), 3.47 (s, 2H), 3.39 (s, 3H), 2.38-2.34 (m, 4H); ¹³C NMR (151 MHz, DMSO d₆) S 156.3, 152.9, 151.1, 141.3, 137.0, 133.4, 133.1, 130.4, 130.3, 129.3 (2C), 126.9, 126.8, 125.7, 124.6 (2C), 103.3, 98.6, 66.2 (2C), 62.1, 53.2 (2C), 52.2, 37.5; IR (neat, cm⁻¹): 3209, 3118, 2957, 2846, 1568, 1403, 1322, 1119, 1008, 937, 864, 763, 531; HRMS (ASAP+, m z): found 448.1903, calcd for C₂₅H₂₇N₅OCl, [M+H]⁺, 448.1904.

### N-(2-Chlorobenzyl)-N-methyl-6-(4-(morpholinomethyl)phenyl)-7H-pyrrolo[2,3-d]pyrimidin-4-amine (LR-2-091)

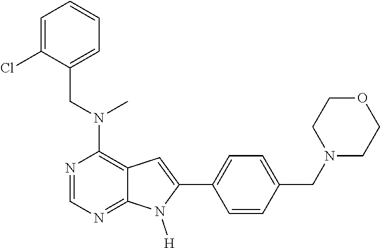 [SMILES: CN(Cc1ccccc1Cl)c1ncnc2[nH]c(-c3ccc(CN4CCOCC4)cc3)cc12]

¹H NMR (600 MHz, DMSO-d₆) δ 12.19 (s, 1H), 8.11 (s, 1H), 7.79 (d, J=8.0 Hz, 2H), 7.51 (dd, J=7.6, 1.6 Hz, 1H), 7.34 (d, J=8.0 Hz, 2H), 7.31-7.26 (m, 2H), 7.12 (dd, J=7.6, 1.8 Hz, 1H), 6.98 (s, 1H), 5.07 (s, 2H), 3.58 (t, J=4.6 Hz, 4H), 3.47-3.46 (m, 5H), 2.38-2.33 (m, 4H); ¹³C NMR (151 MHz, DMSO-d₆) δ 156.3, 152.9, 151.1, 137.1, 135.5, 133.4, 132.0, 130.2, 129.4, 129.4 (2C), 128.5, 127.6, 127.4, 124.6 (2C), 103.4, 98.4, 66.2 (2C), 62.1, 53.2 (2C), 51.3, 38.0; IR (neat, cm⁻¹): 3205, 3116, 2943, 2857, 1561, 1436, 1326, 1111, 1009, 929, 869, 756, 514; HRMS (ASAP+, m z): found 448.1896, calcd. C₂₅H₂₇N₅OCl, [M+H]⁺, 448.1904.

### N-(2,6-Dichlorobenzyl)-N-methyl-6-(4-(morpholinomethyl)phenyl)-7H-pyrrolo[2,3-d]pyrimidin-4-amine (LR-2-094)

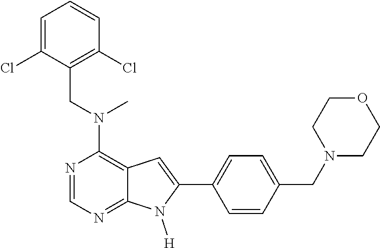 [SMILES: CN(Cc1c(Cl)cccc1Cl)c1ncnc2[nH]c(-c3ccc(CN4CCOCC4)cc3)cc12]

¹H NMR (600 MHz, DMSO-d₆) δ 12.18 (s, 1H), 8.19 (s, 1H), 7.84 (d, J=8.3 Hz, 2H), 7.57-7.53 (m, 2H), 7.45-7.39 (m, 1H), 7.34 (d, J=8.3 Hz, 2H), 7.16 (s, 1H), 5.30 (s, 2H), 3.57 (t, J=4.6 Hz, 4H), 3.47 (s, 2H), 3.10 (s, 3H), 2.39-2.33 (m, 4H); ¹³C NMR (151 MHz, DMSO-d₆) δ 156.6, 153.0, 150.7, 137.0, 136.0 (2C), 133.3, 132.5, 130.5, 130.3, 129.3 (2C), 128.9 (2C), 124.6 (2C), 103.8, 98.7, 66.2 (2C), 62.1, 53.2 (2C), 47.5, 34.7; IR (neat, cm⁻¹): 3222, 3143, 2929, 2865, 1560, 1432, 1316, 1111, 1008, 926, 866, 768, 511; HRMS (ASAP+, m z): found 482.1507, calcd. C₂₅H₂₆N₅OCl₂, [M+H]⁺, 482.1514.

### N-(2,5-Dichlorobenzyl)-N-methyl-6-(4-(morpholinomethyl)phenyl)-7H-pyrrolo[2,3-d]pyrimidin-4-amine (LR-2-123)

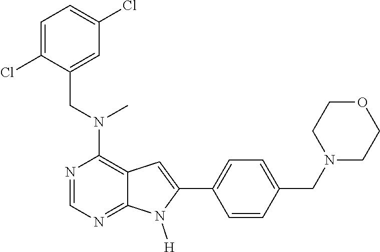 [SMILES: CN(Cc1cc(Cl)ccc1Cl)c1ncnc2[nH]c(-c3ccc(CN4CCOCC4)cc3)cc12]

¹H NMR (600 MHz, DMSO-d₆) δ 12.24 (s, 1H), 8.12 (s, 1H), 7.81 (d, J=8.1 Hz, 2H), 7.56 (d, J=8.5 Hz, 1H), 7.39 (dd, J=8.5, 2.6 Hz, 1H), 7.35 (d, J=8.1 Hz, 2H), 7.09 (d, J=2.6 Hz, 1H), 7.03 (s, 1H), 5.04 (s, 2H), 3.58 (t, J=4.6 Hz, 4H), 3.49 (s, 3H), 3.48 (s, 2H), 2.40-2.34 (m, 4H); ¹³C NMR (151 MHz, DMSO-d₆) δ 156.2, 152.9, 151.0, 138.1, 137.1, 133.6, 132.1, 131.2, 130.7, 130.2, 129.4 (2C), 128.4, 127.1, 124.6 (2C), 103.4, 98.4, 66.2 (2C), 62.1, 53.2 (2C), 51.3, 38.1; IR (neat, cm⁻¹): 3120, 2954, 2851, 1574, 1458, 1321, 1118, 929, 761, 515; HRMS (ASAP+, m/z): found 482.1510, calcd for C₂₅H₂₆N₅OCl₂, [M+H]⁺, 482.1514.

### N-(Cyclohexylmethyl)-N-methyl-6-phenyl-7H-pyrrolo[2,3-d]pyrimidin-4-amine (FAB 04-15)

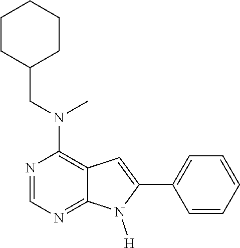 [SMILES: CN(CC1CCCCC1)c1ncnc2[nH]c(-c3ccccc3)cc12]

¹H NMR (400 MHz, DMSO-d₆) δ 12.11 (s, 1H), 8.10 (s, 1H), 7.90-7.83 (m, 2H), 7.42 (t, J=7.8 Hz, 2H), 7.32-7.23 (m, 1H), 7.05 (s, 1H), 3.64 (d, J=7.4 Hz, 2H), 3.38 (s, 3H), 1.82 (s, 1H), 1.66 (d, J=10.8 Hz, 5H), 1.25-1.11 (m, 3H), 1.00 (q, J=11.1 Hz, 2H); ¹³C NMR (101 MHz, DMSO-d₆) δ 156.5, 152.8, 151.1, 132.8, 131.6, 128.8 (2C), 127.2, 124.7 (2C), 103.2, 98.9, 55.8, 40.2, 36.5, 30.3 (2C), 26.1, 25.4 (2C); IR (neat, cm⁻¹): 3099 (m), 2935 (m), 2839 (m), 1565 (s), 1505 (m), 1413 (w), 1300 (s), 1124 (m), 1095 (m), 849 (m), 768 (s); HRMS (ASAP+, m/z): found 321.2084, calcd for C₂₀H₂₅N₄, [M+H]⁺, 321.2079.

### Purine Intermediates

### 6-Chloro-9-(tetrahydro-2H-pyran-2-yl)-9H-purine

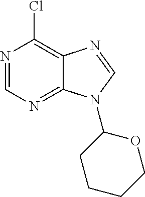 [SMILES: Clc1ncnc2c1ncn2C1CCCCO1]

6-Chloro-9H-purine (1.76 g, 11.4 mmol) and p-toluenesulfonic acid monohydrate (43 mg, 0.227 mmol) in EtOAc (18 mL) was added 3,4-dihydro-2H-pyran (3.1 mL, 34 mmol). The reaction mixture was heated on an oil-bath refluxing for 1.5 h. The heating bath was removed and the reaction mixture let cool to room temperature before the volatiles were removed in vacuo. The crude residue was purified by flash column chromatography (50 g silica-gel cartridge, n-pentane/EtOAc: 1/1, 10 mL/min.) giving 2.39 g (10.0 mmol, 88%) of a white solid; ¹H NMR (400 MHz, CDCl₃) δ: 8.76 (s, 1H), 8.35 (s, 1H), 5.80 (dd, J=10.5, 2.5 Hz, 1H), 4.25-4.16 (m, 1H), 3.85-3.74 (m, 1H), 2.23-2.14 (m, 1H), 2.14-1.99 (m, 2H), 1.89-1.64 (m, 3H).

### 6-Chloro-8-iodo-9-(tetrahydro-2H-pyran-2-yl)-9H-purine

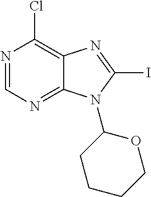 [SMILES: Clc1ncnc2c1nc(I)n2C1CCCCO1]

6-Chloro-9-(tetrahydro-2H-pyran-2-yl)-9H-purine (10.0 g, 41.9 mmol) was dissolved in tetrahydrofuran (140 mL), put under an atmosphere of N₂ and cooled on a bath of dry-ice/acetone. While stirring, lithium diisopropylamine (31.4 mL, 2.0 M in tetrahydrofuran/heptane/ethyl benzene)
was added dropwise over 30 min. The mixture was then stirred for 30 min. Iodine (11.7 g, 46.1 mmol) was dissolved in tetrahydrofuran (50 mL) and added dropwise to the reaction mixture over 30 min. After stirring for another 30 min., sat. aq. NH₄Cl (3 mL) was added and the mixture taken off the cooling bath to let warm to room temperature. The volatiles were removed in vacuo and the oily residue was added water (150 mL) and extracted with CH₂Cl₂ (3×100 mL). The combined organic phases were washed with brine (150 mL), dried using anhydrous Na₂SO₄, filtered and concentrated in vacuo. Purification by silica-gel flash column chromatography (CH₂Cl₂/EtOAc: 9/1) gave 11.4 g (31.2 mmol, 74%) of a pale yellow solid; mp. >120° C. (decomp.); ¹H NMR (400 MHz, CDCl₃) δ: 8.69 (s, 1H), 5.69-5.66 (m, 1H), 4.25-4.20 (m, 1H), 3.79-3.72 (m, 1H), 3.20-3.08 (m, 1H), 2.20-2.12 (m, 1H), 1.94-1.62 (m, 4H); ¹³C NMR (101 MHz, CDCl₃) δ: 152.5, 151.6, 149.5, 134.3, 106.5, 87.4, 69.4, 28.8, 24.6, 23.3; HRMS (ASAP+, m/z): found 280.9093, calcd for C₅H₃N₄CII, [M+H-THP]⁺, 280.9091.

### General Procedure for Amination of THP-Protected Purines

6-Chloro-8-iodo-9-(tetrahydro-2H-pyran-2-yl)-9H-purine (1.0 equiv.), N,N-diisopropylethylamine (1.5 equiv.) and the appropriate amine (1.5 equiv.) were dissolved in 1,4-dioxane (7.5 mL/mmol). The reaction mixture was lowered into an oil-bath set at 70° C. and stirred. Upon reaction completion, the reaction vessel was raised from the oil-bath and allowed to cool for 5 min. before the volatiles were removed in vacuo. The residue was added water (10 mL) and extracted with CH₂Cl₂ (3×10 mL). The combined organic layers were washed with brine (10 mL), dried with anhydrous Na₂SO₄ and filtered. The organic solvent was removed under reduced pressure and the crude product was purified by silica-gel column chromatography.

### Examples

### 8-Iodo-N-(4-methoxybenzyl)-N-methyl-9-(tetrahydro-2H-pyran-2-yl)-9H-purin-6-amine

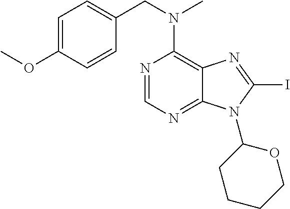 [SMILES: COc1ccc(CN(C)c2ncnc3c2nc(I)n3C2CCCCO2)cc1]

The material was synthesized according to general procedure using N-methyl-2-methoxybenzylamine. Purification by silica-gel flash column chromatography (n-pentane/EtOAc: 2/1, R_{f}=0.26) gave 227 mg (0.47 mmol, 86%) of a colourless solid; mp. 130.5-132.5° C.; ¹H NMR (400 MHz, CDCl₃) δ: 8.30 (s, 1H), 7.21-7.19 (m, 2H), 6.85-6.82 (m, 2H), 5.68-5.65 (m, 1H), 5.21 (br. s, 2H), 4.25-4.21 (m, 1H), 3.79 (s, 3H), 3.77-3.70 (m, 1H), 3.38 (br. s., 3H), 3.15-3.05 (m, 1H), 2.13-2.09 (m, 1H), 1.93-1.68 (m, 3H), 1.63-1.59 (m, 1H); ¹³C NMR (101 MHz, CDCl₃) δ: 158.9, 153.4, 152.2, 148.5, 129.8, 129.1 (2C), 123.3, 113.9 (2C), 94.5, 86.3, 69.3, 55.3, 52.8, 35.9, 29.1, 24.8, 23.5; HRMS (ASAP+, m/z): found 480.0900, calcd for C₁₉H₂₃IN₅O₂, [M+H]⁺, 480.0891.

### General Procedure Suzuki-Cross Coupling of THP Protected Purines

The THP protected purine (1.0 equiv.), aryl boronic acid or pinacol ester (1.0-1.2 equiv.), PdCl₂dppf (2-5 mol %) and potassium carbonate (3.0 equiv.) are charged in an appropriate reaction vessel. The atmosphere is evacuated and back-filled with N₂ three times before adding degassed 1,4-dioxane (6 mL/mmol starting material) and degassed water (3 mL/mmol starting material). The reaction vessel is lowered into an oil-bath set at 60-100° C. and stirred vigorously. Upon reaction completion, the reaction vessel is raised from the oil-bath and allowed to cool for 5 min. before the reaction mixture is transferred to a round-bottomed flask and the volatiles are removed by rotary evaporation. The residue is added water (20 mL/mmol starting material) and extracted with CH₂Cl₂ (3×20 mL/mmol starting material). The combined organic layers are washed with brine (20 mL/mmol), dried with anhydrous Na₂SO₄ and filtered. The organic solvent is removed under reduced pressure and the crude product is purified by silica-gel column chromatography

### Examples

### N-(3-Methylbenzyl)-N-methyl-8-(4-(hydroxymethyl)phenyl)-9-(tetrahydro-2H-pyran-2-yl)-9H-purin-6-amine

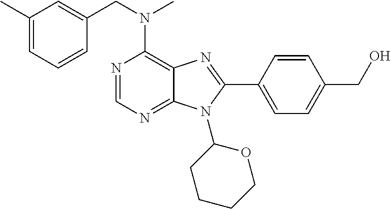 [SMILES: Cc1cccc(CN(C)c2ncnc3c2nc(-c2ccc(CO)cc2)n3C2CCCCO2)c1]

The reaction was performed as described in the general procedure, but using Pd-PEPPSI-SIPr as catalyst. This gave after flash column chromatography (10 g silica-gel cartridge, n-pentane/EtOAc: 1/1) 82 mg (0.18 mmol, 86%) of a colorless solid; mp. 95-101° C. (decomp.); ¹H NMR (400 MHz, CDCl₃) δ: 8.42 (s, 1H), 7.87-7.80 (m, 2H), 7.53-7.46 (m, 2H), 7.23-7.15 (m, 1H), 7.12-7.03 (m, 3H), 5.61 (dd, J=11.3, 2.3 Hz, 1H), 5.35 (br s, 2H), 4.79 (d, J=5.8 Hz, 2H), 3.76-3.65 (m, 1H), 3.45 (br s, 3H), 3.04-2.89 (m, 1H), 2.31 (s, 3H), 2.07-1.95 (m, 1H), 1.85-1.73 (m, 2H), 1.69-1.61 (m, 1H), 1.60-1.51 (m, 2H); ¹³C NMR (151 MHz, CDCl₃) δ: 154.8, 152.3, 152.2, 148.9, 142.6, 138.2, 138.0 130.2, 130.1 (2C), 128.39, 128.38, 127.9, 126.9 (2C), 124.8, 120.0, 83.8, 68.9, 64.9, 53.7, 36.3, 28.6, 24.8, 23.6, 21.5; HRMS (ASAP+, m/z): found 444.2398, calcd for C₂₆H₃₀N₅O₂, [M+H]⁺, 444.2400.

### (4-(6-(Methyl((tetrahydro-2H-pyran-4-yl)methyl)amino)-9-(tetrahydro-2H-pyran-2-yl)-9H-purin-8-yl)phenyl)methanol

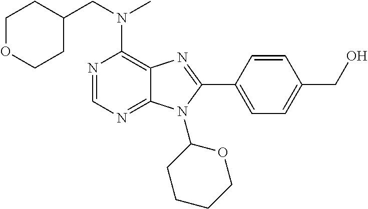 [SMILES: CN(CC1CCOCC1)c1ncnc2c1nc(-c1ccc(CO)cc1)n2C1CCCCO1]

Following the general procedure and purification by flash column chromatography (40×150 mm silica-gel cartridge, EtOAc/1,4-dioxane: 10/1, 10 mL/min.) yielded 1.82 g (4.15 mmol, 92%) of a white solid; mp. 175-178° C.; ¹H NMR (400 MHz, CDCl₃) δ: 8.38 (s, 1H), 7.88-7.81 (m, 2H), 7.55-7.48 (m, 2H), 5.60 (dd, J=11.3, 2.3 Hz, 1H), 4.81 (d, J=5.3 Hz, 2H), 4.30-4.21 (m, 1H), 4.01-3.92 (m, 2H), 3.75-3.64 (m, 1H), 3.41-3.30 (m, 2H), 3.02-2.88 (m, 1H), 2.19-2.06 (m, 1H), 2.02-1.94 (m, 1H), 1.90 (t, J=5.8 Hz, 1H), 1.84-1.72 (m, 1H), 1.67-1.52 (m, 5H), 1.52-1.39 (m, 2H). Missing signals: NCH₂, NCH₃; ¹³C NMR (101 MHz, CDCl₃) δ154.7, 152.1, 152.1, 148.7, 142.6, 130.2, 130.0 (2C), 126.9 (2C), 119.9, 83.8, 68.9, 67.8 (2C), 64.9, 34.6, 30.5 (2C), 28.6, 24.8, 23.5. Missing signals: NCH₂, NCH₃; HRMS (ES+, m/z): found 438.2509, calcd for C₂₄H₃₂N₅O₃, [M+H]⁺, 438.2505.

### N-(4-Methoxybenzyl)-N-methyl-8-(4-(hydroxymethyl)phenyl)-9-(tetrahydro-2H-pyran-2-yl)-9H-purin-6-amine

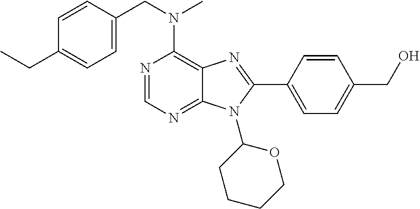 [SMILES: CCc1ccc(CN(C)c2ncnc3c2nc(-c2ccc(CO)cc2)n3C2CCCCO2)cc1]

The reaction was performed as described in the general procedure, but using Pd-PEPPSI-SIPr as catalyst. This gave after flash column chromatography (CH₂Cl₂/EtOAc, 1/1, R_{f}=0.21) 91 mg (0.20 mmol, 95%) of a colourless solid; mp. 95-105° C.; ¹H NMR (400 MHz, CDCl₃) δ: 8.42 (s, 1H), 7.88-7.80 (m, 2H), 7.55-7.46 (m, 2H), 7.27-7.20 (m, 2H), 6.88-6.80 (m, 2H), 5.61 (dd, J=11.3, 2.3 Hz, 1H), 5.32 (br. s, 2H), 4.79 (d, J=5.8 Hz, 2H), 4.29-4.22 (m, 1H), 3.78 (s, 3H), 3.76-3.64 (m, 1H), 3.41 (br. s, 3H), 3.03-2.88 (m, 1H), 2.03-1.93 (m, 1H), 1.84-1.73 (m, 2H), 1.68-1.61 (m, 1H), 1.60-1.50 (m, 2H); ¹³C NMR (101 MHz, CDCl₃) δ: 158.9, 154.6, 152.3, 152.2, 148.9, 142.6, 130.16, 130.14, 130.06 (2C), 129.1 (2C), 126.89 (2C), 120.0, 113.9 (2C), 83.8, 68.9, 64.9, 55.3, 28.6, 24.8, 23.5; HRMS (ASAP+, m/z): found 460.2342, calcd for C₂₆H₃₀N₅O₃, [M+H]⁺, 460.2343.

### General Procedure for Deprotection of THP-Purines

THP-protected starting material (1.0 equiv.) and p-toluenesulfonic acid monohydrate (5 mol %) was dissolved in MeOH (10 mL/mmol) and the reaction mixture was lowered into an oil-bath set at 70° C. and stirred. Upon reaction completion, the reaction vessel was raised from the oil-bath and allowed to cool for 5 min. before the reaction mixture was transferred to a round-bottomed flask and added a mixture of CH₂Cl₂ and MeOH (˜1:1) until all precipitated material had dissolved. To the solution, celite (10:1—celite to starting material, by weight) was added and the volatiles were removed in vacuo. The dry residue was applied to a silica gel column for chromatographic purification.

### Spectroscopic Data Purine Inhibitors

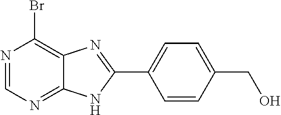 [SMILES: OCc1ccc(-c2nc3c(Br)ncnc3[nH]2)cc1]

### N-Methyl-N-(2-fluorobenzyl)-8-(4-(hydroxymethyl)phenyl)-9H-purin-6-amine (TIA02-130)

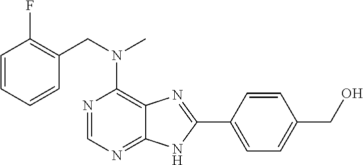 [SMILES: CN(Cc1ccccc1F)c1ncnc2[nH]c(-c3ccc(CO)cc3)nc12]

¹H NMR (600 MHz, DMSO-d₆) δ: 13.36 (s, 1H), 8.24 (s, 1H), 8.09-8.04 (m, 2H), 7.46-7.42 (m, 2H), 7.34-7.29 (m, 1H), 7.29-7.24 (m, 1H), 7.25-7.19 (m, 1H), 7.15-7.10 (m, 1H), 5.56 (br. s, 2H), 5.29 (t, J=5.7 Hz, 1H), 4.55 (d, J=5.5 Hz, 2H), 3.40 (br. s, 3H); ¹³C NMR (151 MHz, DMSO-d₆) δ: 160.5 (d, ¹J_{CF}=244 Hz, 1C), 153.6, 152.9, 151.8, 147.3, 144.5, 129.04, 129.01, 128.98, 128.0, 126.7 (2C), 125.9 (2C), 124.6 (d, ⁴J_{CF}=3.3 Hz, 1C), 120.0, 115.3 (d, ²J_{CF}=21.4 Hz, 1C), 62.5. Missing signals: NCH₃, NCH₂; HRMS (ASAP+, m/z): found 364.1576, calcd for C₂₀H₁₉N₅OF, [M+H]⁺, 364.1574.

### N-Methyl-N-(2-methylbenzyl)-8-(4-(hydroxymethyl)phenyl)-9H-purin-6-amine (TIA03-136)

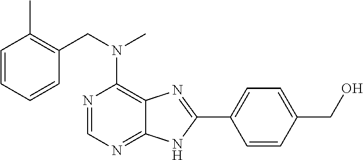 [SMILES: Cc1ccccc1CN(C)c1ncnc2[nH]c(-c3ccc(CO)cc3)nc12]

¹H NMR (600 MHz, DMSO-d₆) δ: 13.45 (s, 1H), 8.22 (s, 1H), 8.07-8.03 (m, 2H), 7.46-7.42 (m, 2H), 7.23-7.19 (m, 1H), 7.17-7.13 (m, 1H), 7.13-7.09 (m, 1H), 7.06-7.01 (m, 1H), 5.37 (br. s, 2H), 5.28 (t, J=5.7 Hz, 1H), 4.55 (d, J=5.5 Hz, 2H), 3.42 (br. s, 3H), 2.33 (s, 3H); ¹³C NMR (151 MHz, DMSO-d₆) δ: 153.8, 152.8, 151.9, 147.1, 144.5, 135.9, 135.8, 130.2, 127.9, 126.7, 126.7 (2C), 126.2, 125.92, 125.89 (2C), 119.9, 62.5, 18.8; HRMS (ASAP+, m/z): found 360.1826, calcd for C₂₁H₂₂N₅O, [M+H]⁺, 360.1824.

### N-(2-Methoxybenzyl)-N-methyl-8-(4-(hydroxymethyl)phenyl)-9H-purin-6-amine (TIA03-106)

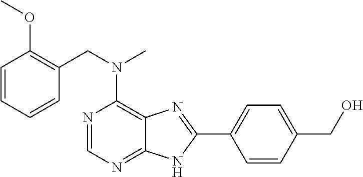 [SMILES: COc1ccccc1CN(C)c1ncnc2[nH]c(-c3ccc(CO)cc3)nc12]

¹H NMR (600 MHz, DMSO-d₆) δ: 13.43 (s, 1H), 8.20 (s, 1H), 8.05 (s, 2H), 7.48-7.39 (m, 2H), 7.25-7.20 (m, 1H), 7.07-7.01 (m, 2H), 6.87-6.82 (m, 1H), 5.28 (s, 1H), 4.55 (s, 2H), 3.84 (s, 3H); ¹³C NMR (151 MHz, DMSO-d₆) δ: 157.1, 153.8, 152.7, 151.8, 147.0, 144.5, 128.0, 128.0, 126.9, 126.7 (2C), 125.9 (2C), 120.3, 120.0, 110.8, 62.5, 55.4; HRMS (ASAP+, m/z): found 376.1773, calcd for C₂₁H₂₂N₅O₂, [M+H]⁺, 376.1773.

### N-Methyl-N-(pyridin-2-ylmethyl)-8-(4-(hydroxymethyl)phenyl)-9H-purin-6-amine (TIA02-164)

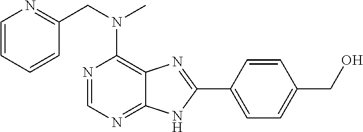 [SMILES: CN(Cc1ccccn1)c1ncnc2[nH]c(-c3ccc(CO)cc3)nc12]

¹H NMR (400 MHz, DMSO-d₆) δ: 13.24 (s, 1H), 8.57-8.47 (m, 1H), 8.22 (s, 1H), 8.13-7.99 (m, 2H), 7.77-7.69 (m, 1H), 7.49-7.40 (m, 2H), 7.32-7.22 (m, 2H), 5.29 (t, J=5.7 Hz, 1H), 4.55 (d, J=5.6 Hz, 2H). Missing signals: NCH₃, NCH₂; ¹³C NMR (151 MHz, DMSO-d₆) δ: 158.0, 153.8, 152.8, 151.8, 149.2, 147.2, 144.5, 136.8, 127.9, 126.8 (2C), 125.9 (2C), 122.2, 121.3, 120.0, 62.5. Missing signals: NCH₃, NCH₂; HRMS (ASAP+, m/z): found 347.1623, calcd for C₁₉H₁₉N₆O, [M+H]⁺, 347.1620.

### (4-(6-(Benzyl(methyl)amino)-9H-purin-8-yl)phenyl)methanol (TIA070)

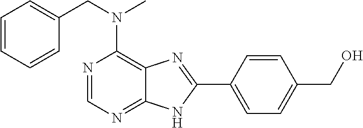 [SMILES: CN(Cc1ccccc1)c1ncnc2[nH]c(-c3ccc(CO)cc3)nc12]

¹H NMR (600 MHz, DMSO-d₆) δ: 13.49 (s, 1H), 8.24 (s, 1H), 8.09-8.05 (m, 2H), 7.47-7.42 (m, 2H), 7.36-7.29 (m, 4H), 7.29-7.22 (m, 1H), 5.29 (t, J=5.7 Hz, 1H), 4.55 (d, J=5.6 Hz, 2H). Missing signals: NCH₂, NCH₃; ¹³C NMR (151 MHz, DMSO-d₆) δ: 153.6, 152.8, 151.9, 147.2, 144.5, 138.3, 128.5 (2C), 127.9, 127.5 (2C), 127.0, 126.8 (2C), 125.9 (2C), 119.9, 62.5, 52.7, 35.6; HRMS (ES+, m/z): found 351.2184, calcd for C₂₁H₂₇N₄O, [M+H]⁺, 351.2185.

### (4-(6-(Benzylamino)-9H-purin-8-yl)phenyl)methanol (TIA098)

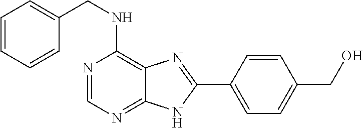 [SMILES: OCc1ccc(-c2nc3c(NCc4ccccc4)ncnc3[nH]2)cc1]

¹H NMR (600 MHz, DMSO-d₆) δ: 13.38 (s, 1H), 8.26 (s, 1H), 8.18 (s, 1H), 8.12-8.08 (m, 2H), 7.49-7.45 (m, 2H), 7.40-7.36 (m, 2H), 7.33-7.27 (m, 2H), 7.24-7.18 (m, 1H), 5.30 (t, J=5.7 Hz, 1H), 4.73 (s, 2H), 4.57 (d, J=5.3 Hz, 2H), 3.56 (s, 1H); ¹³C NMR (151 MHz, DMSO-d₆) δ: 153.8, 152.4, 151.1, 148.2, 144.5, 140.3, 128.2 (2C), 128.0, 127.3 (2C), 126.8 (2C), 126.6, 126.0 (2C), 119.9, 62.5, 42.9; HRMS (ES+, m/z): found 332.1515, calcd for C₁₉H₁₈N₅O, [M+H]⁺, 332.1511.

### N-Methyl-N-(3-fluorobenzyl)-8-(4-(hydroxymethyl)phenyl)-9H-purin-6-amine (TIA02-132)

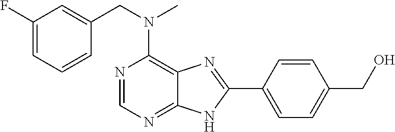 [SMILES: CN(Cc1cccc(F)c1)c1ncnc2[nH]c(-c3ccc(CO)cc3)nc12]

¹H NMR (600 MHz, DMSO-d₆) δ: 13.50 (s, 1H), 8.24 (s, 1H), 8.10-8.05 (m, 2H), 7.47-7.42 (m, 2H), 7.41-7.34 (m, 1H), 7.18-7.12 (m, 2H), 7.12-7.05 (m, 1H), 5.46 (br. s, 2H), 5.29 (t, J=5.7 Hz, 1H), 4.55 (d, J=5.6 Hz, 2H), 3.41 (br. s, 3H); ¹³C NMR (151 MHz, DMSO-d₆) δ: 162.3 (d, ¹J_{CF}=243.7 Hz, 1C), 153.5, 153.0, 151.8, 147.4, 144.5, 141.5, 130.5 (d, ³J_{CF}=8.3 Hz, 1C), 128.0, 126.8 (2C), 125.9 (2C), 123.4 (d, ⁴J_{CF}=2.6 Hz, 1C), 119.9, 114.1 (d, ²J_{CF}=21.5 Hz, 1C), 113.8 (d, ²J_{CF}=20.9 Hz, 1C), 62.5. Missing signals: NCH₃, NCH₂; HRMS (ASAP+, m/z): found 364.1574, calcd for C₂₀H₁₉N₅OF, [M+H]⁺, 364.1574.

### N-Methyl-N-(3-methylbenzyl)-8-(4-(hydroxymethyl)phenyl)-9H-purin-6-amine (TIA02-172)

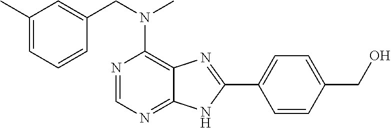 [SMILES: Cc1cccc(CN(C)c2ncnc3[nH]c(-c4ccc(CO)cc4)nc23)c1]

¹H NMR (600 MHz, DMSO-d₆) δ 13.47 (s, 1H), 8.23 (s, 1H), 8.09-8.05 (m, 2H), 7.47-7.42 (m, 2H), 7.23-7.18 (m, 1H), 7.14 (s, 1H), 7.11-7.05 (m, 2H), 5.28 (t, J=5.7 Hz, 1H), 4.55 (d, J=5.5 Hz, 2H), 2.26 (s, 3H); ¹³C NMR (151 MHz, DMSO-d₆) δ 153.6, 152.8, 151.9, 147.1, 144.5, 138.2, 137.6, 128.4, 128.0, 127.9, 127.7, 126.8, 125.9, 124.5, 119.9, 62.5, 21.1; HRMS (ASAP+, m/z): found 360.1825, calcd for C₂₁H₂₂N₅O, [M+H]⁺, 360.1824.

### N-(3-Methoxybenzyl)-N-methyl-8-(4-(hydroxymethyl)phenyl)-9H-purin-6-amine (TIA03-014)

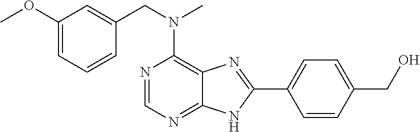 [SMILES: COc1cccc(CN(C)c2ncnc3[nH]c(-c4ccc(CO)cc4)nc23)c1]

¹H NMR (400 MHz, DMSO-d₆) δ 13.46 (br. s, 1H), 8.23 (s, 1H), 8.09-8.07 (m, 2H), 7.45-7.43 (m, 2H), 7.26-7.22 (m, 1H), 6.92-6.85 (m, 2H), 6.85-6.80 (m, 1H), 5.39 (br. s, 2H), 5.29 (t, J=5.6 Hz, 1H), 4.55 (d, J=5.4 Hz, 2H), 3.69 (s, 3H), 3.41 (br. s, 3H); ¹³C NMR (101 MHz, DMSO-d₆) δ159.4, 153.6, 152.9, 151.9, 147.2, 144.6, 140.0, 129.6, 127.9, 126.8 (2C), 126.0 (2C), 119.9, 119.6, 113.4, 112.2, 62.5, 54; HRMS (ASAP+, m/z): found 376.1767, calcd for C₂₁H₂₂N₅O₂, [M+H]⁺, 376.1773.

### N-Methyl-N-(pyridin-3-ylmethyl)-8-(4-(hydroxymethyl)phenyl)-9H-purin-6-amine (TIA03-016)

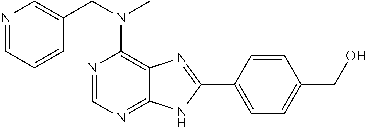 [SMILES: CN(Cc1cccnc1)c1ncnc2[nH]c(-c3ccc(CO)cc3)nc12]

¹H NMR (600 MHz, DMSO-d₆) δ: 13.52 (s, 1H), 8.61-8.58 (m, 1H), 8.49-8.45 (m, 1H), 8.25 (s, 1H), 8.10-8.06 (m, 2H), 7.75-7.70 (m, 1H), 7.47-7.42 (m, 2H), 7.37-7.32 (m, 1H), 5.44 (br. s, 2H), 5.29 (t, J=5.7 Hz, 1H), 4.55 (d, J=5.6 Hz, 2H), 3.42 (br. s, 3H); ¹³C NMR (151 MHz, DMSO-d₆) δ: 153.5, 153.0, 151.8, 149.0, 148.4, 147.4, 144.6, 135.3, 133.9, 127.9, 126.8 (2C), 126.0 (2C), 123.7, 119.9, 62.5. Missing signals: NCH₃, NCH₂; HRMS (ASAP+, m/z): found 347.1615, calcd for C₁₉H₁₉N₆O, [M+H]⁺, 347.1620.

### N-Methyl-N-(oxan-4-ylmethyl)-8-(4-(hydroxymethyl)phenyl)-9H-purin-6-amine (TIA04-051)

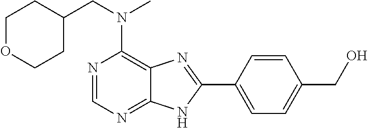 [SMILES: CN(CC1CCOCC1)c1ncnc2[nH]c(-c3ccc(CO)cc3)nc12]

¹H NMR (600 MHz, DMSO-d₆) δ: 13.35 (s, 1H), 8.19 (s, 1H), 8.12-8.05 (m, 2H), 7.49-7.41 (m, 2H), 5.29 (t, J=5.7 Hz, 1H), 4.56 (d, J=5.6 Hz, 2H), 3.86-3.80 (m, 2H), 3.29-3.22 (m, 2H), 2.17-2.06 (m, 1H), 1.61-1.51 (m, 2H), 1.40-1.27 (m, 2H). Missing signals: NCH₃, NCH₂; ¹³C NMR (151 MHz, DMSO-d₆): δ153.8, 152.6, 151.8, 146.8, 144.4, 128.1, 126.8 (2C), 125.8 (2C), 119.8, 66.7 (2C), 62.5, 55.3, 36.5, 34.1, 30.2 (2C); HRMS (ASAP+, m/z): found 354.1930, calcd for C₁₉H₂₄N₅O₂, [M+H]⁺, 354.1930.

### N-Methyl-N-(4-fluorobenzyl)-8-(4-(hydroxymethyl)phenyl)-9H-purin-6-amine (TIA02-134)

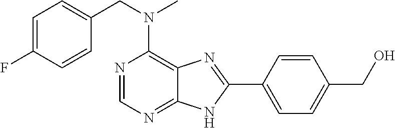 [SMILES: CN(Cc1ccc(F)cc1)c1ncnc2[nH]c(-c3ccc(CO)cc3)nc12]

¹H NMR (600 MHz, DMSO-d₆) δ: 13.50 (s, 1H), 8.24 (s, 1H), 8.09-8.05 (m, 2H), 7.47-7.42 (m, 2H), 7.40-7.35 (m, 2H), 7.19-7.12 (m, 2H), 5.48 (br. s, 2H), 5.29 (t, J=5.7 Hz, 1H), 4.55 (d, J=5.6 Hz, 2H), 3.38 (br. s, 3H); ¹³C NMR (151 MHz, DMSO-d₆) δ: 161.4 (d, ¹J_{CF}=242.7 Hz, 1C), 153.5, 152.9, 151.9, 147.3, 144.5, 134.5, 129.5 (d, ³J_{CF}=8.0 Hz, 2C), 127.9, 126.8 (2C), 125.9 (2C), 119.9, 115.3 (d, ²J_{CF}=21.2 Hz, 2C), 62.5. Missing signals: NCH₃, NCH₂; HRMS (ASAP+, m/z): found 364.1573, calcd for C₂₀H₁₉N₅OF, [M+H]⁺, 364.1574.

### N-Methyl-N-(4-methylbenzyl)-8-(4-(hydroxymethyl)phenyl)-9H-purin-6-amine (TIA02-174)

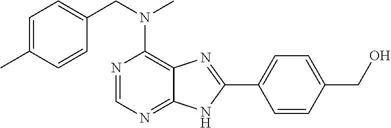 [SMILES: Cc1ccc(CN(C)c2ncnc3[nH]c(-c4ccc(CO)cc4)nc23)cc1]

¹H NMR (600 MHz, DMSO-d₆) δ: 13.48 (s, 1H), 8.23 (s, 1H), 8.09-8.05 (m, 2H), 7.46-7.42 (m, 2H), 7.23-7.18 (m, 2H), 7.15-7.10 (m, 2H), 5.53 (br. s, 2H), 5.29 (t, J=5.7 Hz, 1H), 4.55 (d, J=5.6 Hz, 2H), 3.23 (br. s, 3H), 2.26 (s, 3H); ¹³C NMR (151 MHz, DMSO-d₆) δ: 153.6, 152.8, 151.9, 147.1, 144.5, 136.1, 135.2, 129.1 (2C), 128.0, 127.5 (2C), 126.8 (2C), 125.9 (2C), 119.9, 62.5, 20.7; HRMS (ASAP+, m/z): found 360.1823, calcd for C₂₁H₂₂N₅O, [M+H]⁺, 360.1824

### N-(4-Methoxybenzyl)-N-methyl-8-(4-(hydroxymethyl)phenyl)-9H-purin-6-amine (TIA03-078)

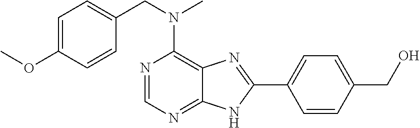 [SMILES: COc1ccc(CN(C)c2ncnc3[nH]c(-c4ccc(CO)cc4)nc23)cc1]

¹H NMR (600 MHz, DMSO-d₆) δ: 13.43 (s, 1H), 8.23 (s, 1H), 8.10-8.06 (m, 2H), 7.47-7.42 (m, 2H), 7.29-7.24 (m, 2H), 6.91-6.86 (m, 2H), 5.46 (br. s, 2H), 5.28 (t, J=5.7 Hz, 1H), 4.55 (d, J=5.6 Hz, 2H), 3.71 (s, 3H), 3.28 (br. s, 3H); ¹³C NMR (151 MHz, DMSO-d₆) δ: 158.4, 153.5, 152.8, 151.9, 147.1, 144.5, 130.1, 128.9 (2C), 128.0, 126.8 (2C), 125.9 (2C), 119.9, 113.9 (2C), 62.5, 55.0, 52.0; HRMS (ASAP+, m/z): found 376.1766, calcd for C₂₁H₂₂N₅O₂, [M+H]⁺, 376.1773.

### N-Methyl-N-(pyridin-3-ylmethyl)-8-(4-(hydroxymethyl)phenyl)-9H-purin-6-amine (TIA02-166)

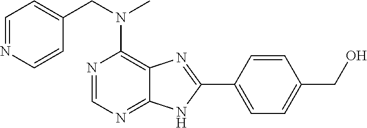 [SMILES: CN(Cc1ccncc1)c1ncnc2[nH]c(-c3ccc(CO)cc3)nc12]

¹H NMR (600 MHz, DMSO-d₆) δ: 13.51 (s, 1H), 8.52-8.48 (m, 2H), 8.24 (s, 1H), 8.08-8.03 (m, 2H), 7.46-7.42 (m, 2H), 7.30-7.26 (m, 2H), 5.46 (br. s, 2H), 5.29 (t, J=5.7 Hz, 1H), 4.55 (d, J=5.7 Hz, 2H), 3.47 (br. s, 3H); ¹³C NMR (151 MHz, DMSO-d₆) δ: 153.6, 152.9, 151.8, 149.7 (2C), 147.5, 147.4, 144.6, 127.9, 126.7 (2C), 126.0 (2C), 122.3 (2C), 119.9, 62.5. Missing signals: NCH₃, NCH₂; HRMS (ASAP+, m/z): found 347.1616, calcd for C₁₉H₁₉N₆O, [M+H]⁺, 347.1620.

### 8-(4-(Hydroxymethyl)phenyl)-6-(2-phenylpyrrolidin-1-yl)-9H-purine (TIA02-176)

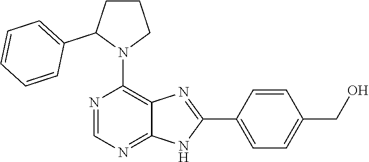 [SMILES: OCc1ccc(-c2nc3c(N4CCCC4c4ccccc4)ncnc3[nH]2)cc1]

¹H NMR (600 MHz, DMSO-d₆) δ: 13.49-13.11 (m, 1H), 8.38-7.78 (m, 3H), 7.57-7.34 (m, 2H), 7.32-7.19 (m, 4H), 7.19-7.08 (m, 1H), 6.49-5.44 (m, 1H), 5.29 (s, 1H), 4.72-4.22 (m, 3H), 4.16-3.71 (m, 1H), 2.48-2.29 (m, 1H), 2.13-1.80 (m, 3H). Significant broadening and conformational splitting of signals; ¹³C NMR (151 MHz, DMSO-d₆): 152.0, 144.3, 128.1, 128.1, 126.7, 126.2, 125.8, 125.6, 62.5, 61.7, 61.0, 49.9, 47.8, 35.0, 33.8, 23.4, 20.7. Significant broadening and conformational splitting of signals (divided signals). Weak and missing signals; HRMS (ASAP+, m/z): found 372.1827, calcd for C₂₂H₂₂N₅O, [M+H]⁺, 372.1824.

### N-Methyl-N-(cyclohexylmethyl)-8-(4-(hydroxymethyl)phenyl)-9H-purin-6-amine (TIA03-024)

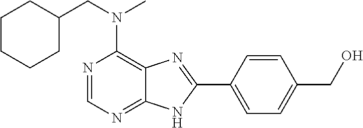 [SMILES: CN(CC1CCCCC1)c1ncnc2[nH]c(-c3ccc(CO)cc3)nc12]

¹H NMR (600 MHz, DMSO-d₆) δ: 13.36 (s, 1H), 8.18 (s, 1H), 8.13-8.03 (m, 2H), 7.52-7.38 (m, 2H), 5.29 (t, J=5.6 Hz, 1H), 4.56 (d, J=5.5 Hz, 2H), 1.97-1.82 (m, 1H), 1.73-1.63 (m, 4H), 1.63-1.55 (m, 1H), 1.27-1.09 (m, 3H), 1.09-0.97 (m, 2H). Missing signals: NCH₃, NCH₂; ¹³C NMR (151 MHz, DMSO-d₆) δ: 153.8, 152.6, 151.8, 146.7, 144.4, 128.2, 126.8 (2C), 125.8 (2C), 119.8, 62.5, 36.6, 30.1 (2C), 26.1, 25.4 (2C). Missing signals: NCH₃, NCH₂; HRMS (ASAP+, m/z): found 352.2139, calcd for C₂₀H₂₈N₅O, [M+H]⁺, 352.2137.

### N-Methyl-N-(thiophen-2-ylmethyl)-8-(4-(hydroxymethyl)phenyl)-9H-purin-6-amine (TIA03-066)

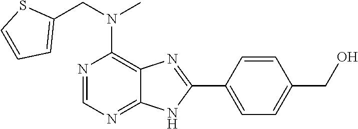 [SMILES: CN(Cc1cccs1)c1ncnc2[nH]c(-c3ccc(CO)cc3)nc12]

¹H NMR (600 MHz, DMSO-d₆) δ: 13.50 (s, 1H), 8.26 (s, 1H), 8.16-8.05 (m, 2H), 7.52-7.43 (m, 2H), 7.43-7.35 (m, 1H), 7.19-7.11 (m, 1H), 7.01-6.94 (m, 1H), 5.64 (br. s, 2H), 4.56 (s, 2H), 3.43 (br. s, 3H); ¹³C NMR (151 MHz, DMSO-d₆) δ 153.0, 152.9, 151.8, 147.4, 144.6, 140.3, 128.0, 126.9, 126.8 (2C), 126.6, 126.0 (2C), 125.9, 120.0, 62.5, 47.8, 35.3; HRMS (ASAP+, m/z): found 352.1236, calcd for C₁₈H₁₈N50S, [M+H]⁺, 352.1232.

### N-Methyl-N-(oxan-2-ylmethyl)-8-(4-(hydroxymethyl)phenyl)-9H-purin-6-amine (TIA04-095)

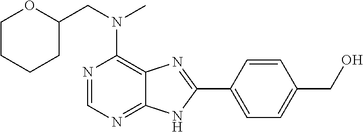 [SMILES: CN(CC1CCCCO1)c1ncnc2[nH]c(-c3ccc(CO)cc3)nc12]

¹H NMR (600 MHz, DMSO-d₆) δ: 13.18 (s, 1H), 8.19 (s, 1H), 8.09-8.05 (m, 2H), 7.48-7.44 (m, 2H), 5.29 (t, J=5.7 Hz, 1H), 4.56 (d, J=5.3 Hz, 2H), 3.90-3.84 (m, 1H), 3.72-3.65 (m, 1H), 3.33-3.25 (m, 1H), 1.81-1.74 (m, 1H), 1.71-1.68 (m, 1H), 1.50-1.38 (m, 3H), 1.30-1.24 (m, 1H); ¹³C NMR (151 MHz, DMSO-d₆) δ: 153.6, 152.6, 151.8, 146.9, 144.4, 128.1, 126.8 (2C), 125.8 (2C), 119.9, 77.5, 67.4, 62.5, 55.3, 37.3, 28.7, 25.6, 22.7; HRMS (ASAP+, m/z): found 354.1931, calcd for C₁₉H₂₄N₅O₂, [M+H]⁺, 354.1930.

### N-Methyl-N-(2-hydroxybenzyl)-8-(4-(hydroxymethyl)phenyl)-9H-purin-6-amine (TIA04-009)

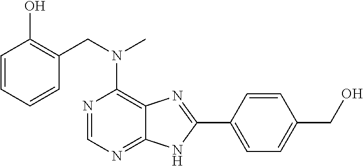 [SMILES: CN(Cc1ccccc1O)c1ncnc2[nH]c(-c3ccc(CO)cc3)nc12]

¹H NMR (600 MHz, DMSO-d₆) δ: 13.47 (s, 1H), 9.94 (s, 1H), 8.22 (s, 1H), 8.10-8.05 (m, 2H), 7.47-7.42 (m, 2H), 7.11-7.05 (m, 2H), 6.86-6.82 (m, 1H), 6.75-6.69 (m, 1H), 5.28 (t, J=5.7 Hz, 1H), 5.03 (br. s, 2H), 4.56 (d, J=5.6 Hz, 2H), 3.64 (br. s, 3H); ¹³C NMR (151 MHz, DMSO-d₆) δ: 155.5, 153.6, 152.8, 151.7, 147.2, 144.5, 128.4, 128.1, 127.9, 126.7 (2C), 125.9 (2C), 123.8, 119.9, 119.0, 115.4, 62.5, 48.1. Missing signals: NCH₃; HRMS (ASAP+, m/z): found 363.1613, calcd for C₂₀H₂₀N₅O₂, [M+H]⁺, 362.1617.

### N-Methyl-N-(3-trifluoromethylbenzyl)-8-(4-(hydroxymethyl)phenyl)-9H-purin-6-amine (TIA03-108)

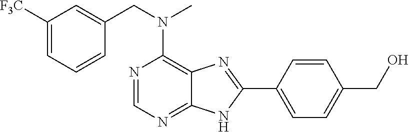 [SMILES: CN(Cc1cccc(C(F)(F)F)c1)c1ncnc2[nH]c(-c3ccc(CO)cc3)nc12]

¹H NMR (600 MHz, DMSO-d₆) δ: 13.52 (s, 1H), 8.25 (s, 1H), 8.13-8.00 (m, 2H), 7.81-7.69 (m, 1H), 7.65-7.60 (m, 2H), 7.59-7.55 (m, 1H), 7.46-7.42 (m, 2H), 5.47 (br. s, 2H), 5.29 (t, J=5.7 Hz, 1H), 4.55 (d, J=5.5 Hz, 2H), 3.47 (br. s, 3H); ¹³C NMR (151 MHz, DMSO-d₆) δ: 153.5, 152.9, 151.9, 147.4, 144.6, 140.0, 131.4, 129.7, 129.2 (q, ²J_{CF}=31.5 Hz, 1C), 127.9, 126.7 (2C), 125.9 (2C), 124.2 (q, ¹J_{CF}=272.1 Hz, 1C), 124.1 (q, ³J_{CF}=3.1 Hz, 1C), 123.8 (q, ³J_{CF}=3.8 Hz, 1C), 119.9, 62.5, 52.4, 36.0; HRMS (ASAP+, m/z): found 414.1543, calcd for C₂₁H₁₉N₅OF₃, [M+H]⁺, 414.1542.

### N-(3,4-Dimethoxybenzyl)-N-methyl-8-(4-(hydroxymethyl)phenyl)-9H-purin-6-amine (TIA03-080)

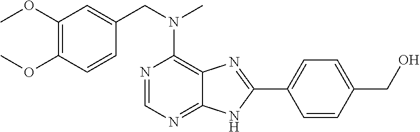 [SMILES: COc1ccc(CN(C)c2ncnc3[nH]c(-c4ccc(CO)cc4)nc23)cc1OC]

¹H NMR (600 MHz, DMSO-d₆) δ: 13.47 (s, 1H), 8.23 (s, 1H), 8.11-8.07 (m, 2H), 7.47-7.43 (m, 2H), 7.03 (s, 1H), 6.90-6.87 (m, 1H), 6.86-6.83 (m, 1H), 5.29 (t, J=5.7 Hz, 1H), 4.55 (d, J=5.3 Hz, 2H), 3.71 (s, 3H), 3.66 (s, 3H); ¹³C NMR (151 MHz, DMSO-d₆) δ: 153.5, 152.9, 151.8, 148.7, 148.0, 147.1, 144.5, 130.6, 128.0, 126.8 (2C), 125.9 (2C), 119.9, 119.8, 111.89, 111.85, 62.5, 55.5, 55.4; HRMS (ASAP+, m/z): found 406.1875, calcd for C₂₂H₂₄N₅O₃, [M+H]⁺, 406.1879.

### N-Methyl-N-(cyclopentylmethyl)-8-(4-(hydroxymethyl)phenyl)-9H-purin-6-amine (TIA03-188)

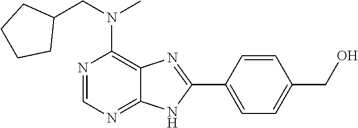 [SMILES: CN(CC1CCCC1)c1ncnc2[nH]c(-c3ccc(CO)cc3)nc12]

¹H NMR (600 MHz, DMSO-d₆) δ: 13.39 (s, 1H), 8.18 (s, 1H), 8.10-8.06 (m, 2H), 7.48-7.43 (m, 2H), 5.30 (t, J=5.7 Hz, 1H), 4.56 (d, J=5.6 Hz, 2H), 2.44-2.38 (m, 1H), 1.71-1.61 (m, 4H), 1.52-1.44 (m, 2H), 1.35-1.28 (m, 2H). Missing signals: NCH₃, NCH₂; ¹³C NMR (151 MHz, DMSO-d₆) δ: 153.7, 152.6, 151.8, 146.7, 144.4, 128.1, 126.8 (2C), 125.8 (2C), 119.8, 62.5, 29.7 (2C), 24.6 (2C). Missing signals: NCH₃, NCH₂, CH (HSQC: δ38.7); HRMS (ASAP+, m/z): found 338.1980, calcd for C₁₉H₂₄N₅O, [M+H]⁺, 338.1981.

### N-Methyl-N-(tetrahydrofuran-2-ylmethyl)-8-(4-(hydroxymethyl)phenyl)-9H-purin-6-amine (TIA04-093)

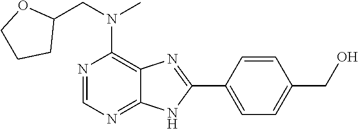 [SMILES: CN(CC1CCCO1)c1ncnc2[nH]c(-c3ccc(CO)cc3)nc12]

¹H NMR (600 MHz, DMSO-d₆) δ: 13.39 (s, 1H), 8.19 (s, 1H), 8.10-8.06 (m, 2H), 7.48-7.44 (m, 2H), 5.29 (t, J=5.7 Hz, 1H), 4.56 (d, J=5.6 Hz, 2H), 4.24-4.19 (m, 1H), 3.81-3.75 (m, 1H), 3.65-3.58 (m, 1H), 2.01-1.94 (m, 1H), 1.94-1.87 (m, 1H), 1.84-1.74 (m, 1H), 1.65-1.58 (m, 1H). Missing signals: NCH₃, NCH₂; ¹³C NMR (151 MHz, DMSO-d₆) δ: 153.6, 152.6, 151.8, 146.9, 144.4, 128.1, 126.8 (2C), 125.8 (2C), 120.0, 77.6, 67.1, 62.5, 54.0, 36.4, 28.6, 25.0; HRMS (ASAP+, m/z): found 340.1774, calcd for C₁₈H₂₂N₅O₂, [M+H]⁺, 340.1773.

### N-Methyl-N-(tetrahydrofuran-3-ylmethyl)-8-(4-(hydroxymethyl)phenyl)-9H-purin-6-amine (TIA04-049)

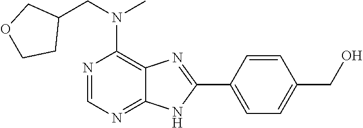 [SMILES: CN(CC1CCOC1)c1ncnc2[nH]c(-c3ccc(CO)cc3)nc12]

¹H NMR (600 MHz, DMSO-d₆) δ: 13.42 (s, 1H), 8.20 (s, 1H), 8.11-8.07 (m, 2H), 7.48-7.44 (m, 2H), 5.30 (t, J=5.8 Hz, 1H), 4.56 (d, J=4.7 Hz, 2H), 3.85-3.79 (m, 1H), 3.79-3.74 (m, 1H), 3.67-3.61 (m, 1H), 3.53-3.48 (m, 1H), 2.75 (app hept, J=7.1 Hz, 1H), 2.00-1.91 (m, 1H), 1.71-1.62 (m, 1H). Missing signals: NCH₃, NCH₂; ¹³C NMR (151 MHz, DMSO-d₆) δ: 153.6, 152.7, 151.8, 147.0, 144.4, 128.1, 126.8 (2C), 125.8 (2C), 119.9, 70.4, 66.9, 62.5, 51.9, 38.2, 36.3, 29.5; HRMS (ASAP+, m/z): found 340.1778, calcd for C₁₈H₂₂N₅O₂, [M+H]⁺, 340.1773.

### 4-(6-(2-Isopropylpyrrolidin-1-yl)-9H-purin-8-yl)phenyl)methanol (TIA04-065)

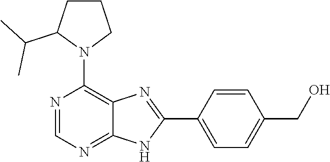 [SMILES: CC(C)C1CCCN1c1ncnc2[nH]c(-c3ccc(CO)cc3)nc12]

¹H NMR (400 MHz, DMSO-d₆) δ: 13.02 (br s, 1H), 8.18 (s, 1H), 8.13-8.06 (m, 2H), 7.49-7.43 (m, 2H), 5.04 (t, J=5.7 Hz, 1H), 4.80 (br s, 1H), 4.58 (d, J=5.2 Hz, 2H), 4.24-4.10 (m, 1H), 3.87-3.82 (m, 1H), 2.50-2.40 (m, 1H), 2.06-1.86 (m, 4H), 0.97 (d, J=6.9 Hz, 3H), 0.86 (d, J=6.8 Hz, 3H); ¹³C NMR (151 MHz, DMSO-d₆) δ: 152.4, 152.0 (2C), 147.2, 144.3, 128.2, 126.8 (2C), 125.8 (2C), 120.0, 62.5, 62.3, 31.0, 19.4, 16.6. Missing signals: NCH₂CH₂CH₂; HRMS (ES+, m/z): found 338.1986, calcd for C₁₉H₂₄N₅O, [M+H]⁺, 338.1981.

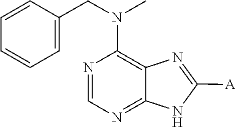

### N-Benzyl-N-methyl-8-phenyl-9H-purin-6-amine (TIA072)

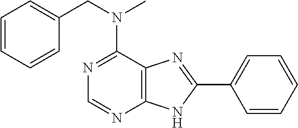 [SMILES: CN(Cc1ccccc1)c1ncnc2[nH]c(-c3ccccc3)nc12]

¹H NMR (400 MHz, DMSO-d₆) δ: 13.54 (br s, 1H), 8.25 (s, 1H), 8.15-8.08 (m, 2H), 7.54-7.44 (m, 3H), 7.35-7.29 (m, 4H), 7.29-7.22 (m, 1H), 5.45 (br s, 2H), 3.43 (br s, 3H); ¹³C NMR (101 MHz, DMSO-d₆) δ: 153.7, 152.9, 152.0, 147.1, 129.9, 129.5, 128.9 (2C), 128.5 (2C), 127.5 (2C), 127.1, 126.1 (2C), 119.9. Missing signals: NCH₂C, NCH₃; HRMS (ES+, m/z): found 316.1565, calcd for C₁₉H₁₈N₅, [M+H]⁺, 316.1562.

### N-Benzyl-N-methyl-9H-purin-6-amine (TIA008)

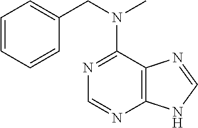 [SMILES: CN(Cc1ccccc1)c1ncnc2[nH]cnc12]

¹H NMR (400 MHz, DMSO-d₆) δ: 13.03 (s, 1H), 8.23 (s, 1H), 8.11 (s, 1H), 7.36-7.27 (m, 2H), 7.28-7.20 (m, 3H), 5.36 (br s, 2H), 3.32 (br s, 3H); ¹³C NMR (101 MHz, DMSO-d₆) δ: 154.1, 151.9, 151.3, 138.2, 138.1, 128.5 (2C), 127.3 (2C), 127.0, 118.8. Missing signals: NCH₂, NCH₃; HRMS (ASAP+, m/z): found 240.1249, calcd for C₁₃H₁₄N₅, [M+H]⁺, 240.1249.

### N-Benzyl-8-phenyl-9H-purin-6-amine (TIA099)

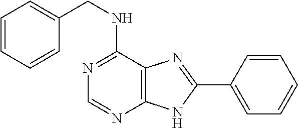 [SMILES: c1ccc(CNc2ncnc3[nH]c(-c4ccccc4)nc23)cc1]

¹H NMR (600 MHz, DMSO-d₆) δ: 13.44 (s, 1H), 8.28 (s, 1H), 8.19 (s, 1H), 8.17-8.13 (m, 2H), 7.56-7.51 (m, 2H), 7.51-7.46 (m, 1H), 7.40-7.36 (m, 2H), 7.33-7.27 (m, 2H), 7.27-7.18 (m, 1H), 4.72 (s, 2H); ¹³C NMR (151 MHz, DMSO-d₆) δ: 153.9, 152.5, 151.2, 148.1, 140.3, 129.81, 129.78, 128.9 (2C), 128.2 (2C), 127.2 (2C), 126.6, 126.1 (2C), 120.0, 42.9; HRMS (ES+, m/z): found 302.1409, calcd for C₁₈H₁₆N₅, [M+H]⁺, 302.1406.

### 2-(6-(Benzyl(methyl)amino)-9H-purin-8-yl)phenol (TIA073)

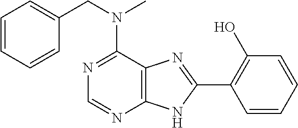 [SMILES: CN(Cc1ccccc1)c1ncnc2[nH]c(-c3ccccc3O)nc12]

¹H NMR (400 MHz, DMSO-d₆) δ: 13.75 (s, 1H), 12.23 (s, 1H), 8.31 (s, 1H), 8.06-7.99 (m, 1H), 7.38-7.22 (m, 6H), 7.02-6.93 (m, 2H), 5.25 (s, 2H), 3.39 (s, 3H); ¹³C NMR (151 MHz, DMSO-d₆) δ: 157.3, 153.1, 152.5, 151.6, 148.1, 137.9, 131.6, 128.6 (2C), 127.1, 127.0 (2C), 126.4, 119.3, 117.3, 117.0, 112.4, 52.9, 36.1; HRMS (ES+, m/z): found 332.1511, calcd for C₁₉H₁₈N₅O, [M+H]⁺, 332.1511.

### (2-(6-(Benzyl(methyl)amino)-9H-purin-8-yl)phenyl)methanol (JKS02-027)

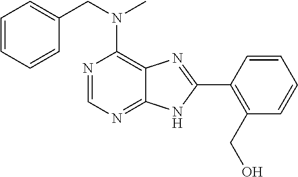 [SMILES: CN(Cc1ccccc1)c1ncnc2[nH]c(-c3ccccc3CO)nc12]

¹H NMR (CDCl₃, 600 MHz) δ: 8.41 (s, 1H), 8.06 (d, J=7.8 Hz, 1H), 7.58-7.56 (m, 1H), 7.50-7.48 (m, 2H), 7.34-7.32 (m, 4H), 7.28-7.27 (m, 1H), 5.20 (s br, 2H), 4.70 (s, 2H), 3.66 (s br, 3H), 1.60 (s br, 1H, OH); ¹³C NMR (150 MHz, CDCl₃) 5: 154.7, 152.2, 151.7, 148.8, 140.7, 137.1, 131.4, 130.5, 129.3, 128.9, 128.7 (2C), 128.5, 127.5 (2C), 120.3, 64.8; The CH₂N and CH₃N carbons were not observed due to dynamic effects; HRMS (APCI/ASAP+, m/z) found 346.1668 (calcd. C₂₀H₂₀N₅O, [M+H]⁺, 346.1666.

### N-Benzyl-N-methyl-8-morpholino-9H-purin-6-amine (TIA04-105)

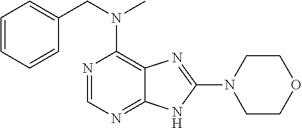 [SMILES: CN(Cc1ccccc1)c1ncnc2[nH]c(N3CCOCC3)nc12]

¹H NMR (600 MHz, DMSO-d₆) δ: 12.20 (s, 1H), 8.04 (s, 1H), 7.33-7.28 (m, 2H), 7.28-7.20 (m, 3H), 5.25-5.22 (m, 2H), 3.71-3.66 (m, 4H), 3.42-3.38 (m, 4H), 3.25 (s, 3H); ¹³C NMR (151 MHz, DMSO-d₆) δ: 152.0 (2C), 151.3, 149.1, 138.8, 128.4 (2C), 127.5 (2C), 126.9, 117.6, 65.4 (2C), 52.4, 46.1 (2C), 35.3; HRMS (ES+, m/z): found 325.0000, calcd for C₁₇H₂₁N₆O [M+H]⁺ 325.0000.

### Purines Various

### N-(Cyclohexylmethyl)-N-methyl-8-(4-(4-morpholinomethyl)phenyl)-9H-purin-6-amine (TIA04-117)

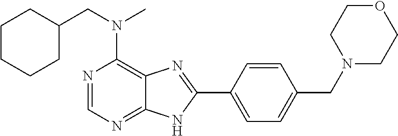 [SMILES: CN(CC1CCCCC1)c1ncnc2[nH]c(-c3ccc(CN4CCOCC4)cc3)nc12]

¹H NMR (600 MHz, DMSO-d₆) δ: 13.36 (s, 1H), 8.18 (s, 1H), 8.10-8.06 (m, 2H), 7.48-7.43 (m, 2H), 3.61-3.56 (m, 4H), 3.52 (s, 2H), 2.40-2.35 (m, 4H), 1.91-1.82 (m, 1H), 1.71-1.65 (m, 4H), 1.62-1.58 (m, 1H), 1.21-1.10 (m, 3H), 1.08-1.01 (m, 2H). Missing signals: N(CH₃)CH₂; ¹³C NMR (151 MHz, DMSO-d₆) δ: 153.8, 152.6, 151.8, 146.5, 139.7, 129.4 (2C), 128.5, 125.8 (2C), 119.8, 66.2 (2C), 62.0, 53.2 (2C), 30.1 (2C), 26.1, 25.4 (2C). Missing signals: N(CH₃)CH₂CH; HRMS (ASAP+, m/z): found 421.2713, calcd for C₂₄H₃₃N₆O, [M+H]⁺, 421.2713.

### 8-(Cyclohex-1-en-1-yl)-N-(cyclohexylmethyl)-N-methyl-9H-purin-6-amine (TIA03-186)

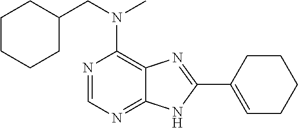 [SMILES: CN(CC1CCCCC1)c1ncnc2[nH]c(C3=CCCCC3)nc12]

¹H NMR (600 MHz, DMSO-d₆) δ: 12.85 (s, 1H), 8.12 (s, 1H), 6.75-6.70 (m, 1H), 2.50-2.45 (m, 2H), 2.23-2.20 (m, 2H), 1.85-1.79 (m, 1H), 1.73-1.56 (m, 9H), 1.21-1.08 (m, 3H), 1.03-0.94 (m, 2H). Missing signals: NCH₂, NCH₃; ¹³C NMR (151 MHz, DMSO-d₆) δ: 153.7, 152.2, 151.6, 148.3, 129.2, 128.1, 119.1, 30.0 (2C), 26.2, 25.4 (2C), 25.0, 24.4, 21.9, 21.6. Missing signals: NCH₂C, NCH₃; HRMS (ES+, m/z): found 326.2339, calcd for C₁₉H₂₈N₅, [M+H]⁺, 326.2345.

### N-Methyl-N-(oxan-4-ylmethyl)-8-(4-(2-hydroxyethyl)phenyl)-9H-purin-6-amine (TIA05-014)

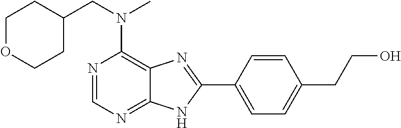 [SMILES: CN(CC1CCOCC1)c1ncnc2[nH]c(-c3ccc(CCO)cc3)nc12]

¹H NMR (600 MHz, DMSO-d₆) δ: 13.37 (s, 1H), 8.19 (s, 1H), 8.05-8.01 (m, 2H), 7.39-7.35 (m, 2H), 4.68 (t, J=5.2 Hz, 1H), 3.86-3.80 (m, 2H), 3.64 (td, J=6.9, 4.5 Hz, 2H), 3.28-3.21 (m, 2H), 2.78 (t, J=6.9 Hz, 2H), 2.16-2.06 (m, 1H), 1.58-1.52 (m, 2H), 1.38-1.28 (m, 2H). Missing signals: N(CH₃)NCH₂; ¹³C NMR (151 MHz, DMSO-d₆) δ: 153.7, 152.6, 151.7, 146.9, 141.6, 129.5 (2C), 127.4, 125.8 (2C), 119.8, 66.7 (2C), 61.9, 55.3, 38.8, 36.2, 34.2, 30.2 (2C); HRMS (ESI+, m/z): found 368.2086, calcd for C₂₀H₂₆N₅O₂, [M+H]⁺, 368.2087.

### N-Methyl-8-phenyl-N-(pyridin-3-ylmethyl)-9H-purin-6-amine (TIA109)

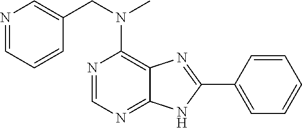 [SMILES: CN(Cc1cccnc1)c1ncnc2[nH]c(-c3ccccc3)nc12]

¹H NMR (400 MHz, CDCl₃) 5: 14.73 (br s, 1H), 8.71-8.66 (m, 1H), 8.60-8.53 (m, 1H), 8.51 (s, 1H), 8.19-8.12 (m, 2H), 7.77-7.70 (m, 1H), 7.58-7.50 (m, 2H), 7.53-7.44 (m, 1H), 7.30-7.22 (m, 1H), 5.51 (br s, 2H), 3.55 (br s, 3H); ¹³C NMR (151 MHz, CDCl₃) 5: 154.5, 152.6, 151.1, 149.6, 148.9, 148.5, 135.6, 133.6, 130.1, 130.0, 129.0 (2C), 126.5 (2C), 123.6, 121.1, 51.5, 36.3; HRMS (ES+, m/z): found 317.1518, calcd for C₁₈H₁₇N₆, [M+H]⁺, 317.1515.

### (R)—N-Methyl-6-phenyl-N-(1-phenylethyl)-7H-pyrrolo[2,3-d]pyrimidin-4-amine (KUL01-093)

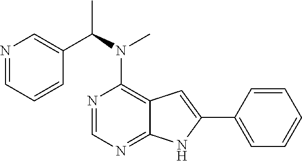 [SMILES: C[C@H](c1cccnc1)N(C)c1ncnc2[nH]c(-c3ccccc3)cc12]

¹H NMR (400 MHz, DMSO-d₆) δ: 12.20 (bs, 1H), 8.17 (s, 1H), 7.90-7.85 (m, 2H), 7.44-7.38 (m, 2H), 7.37-7.32 (m, 4H), 7.30-7.24 (m, 2H), 7.14-7.09 (m, 1H), 6.50-6.42 (m, 1H), 3.08 (s, 3H), 1.59-1.61 (m, 3H); ¹³C NMR (100 MHz, DMSO-d₆) δ: 156.6, 153.1, 151.1, 141.6, 133.1, 131.6, 128.8 (2C), 128.5 (2C), 127.3 (2C), 126.94, 126.89, 124.7 (2C), 103.5, 99.1, 52.1, 31.7, 16.2; HRMS (ASAP+, m/z): found 329.1763, calculated for C₂₁H₂₁N₄, [M+H]⁺, 329.1766; mp. 177-180° C.

### (R)-2-((6-(4-(Hydroxymethyl)phenyl)-7H-pyrrolo[2,3-d]pyrimidin-4-yl)(methyl)amino)-2-phenylethan-1-ol (TIA05-010)

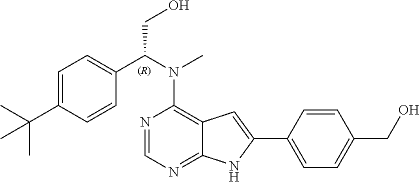

¹H NMR (600 MHz, DMSO-d₆) δ: 12.12 (s, 1H), 8.14 (s, 1H), 7.82 (d, J=8.0 Hz, 2H), 7.38-7.32 (m, 6H), 7.28-7.24 (m, 1H), 7.07 (s, 1H), 6.29 (s, 1H), 5.19 (t, J=5.7 Hz, 1H), 4.99 (t, J=5.3 Hz, 1H), 4.51 (d, J=5.6 Hz, 2H), 4.11-4.05 (m, 1H), 4.02-3.96 (m, 1H), 3.21 (s, 3H); HRMS (ASAP+, m/z): found 375.1820, calcd for C₂₂H₂₃N₄O₂, [M+H]⁺, 375.1821.

### (R)-(4-(4-((1-(4-(tert-butyl)phenyl)ethyl)(methyl)amino)-7H-pyrrolo[2,3-d]pyrimidin-6-yl)phenyl)methanol (TIA097)

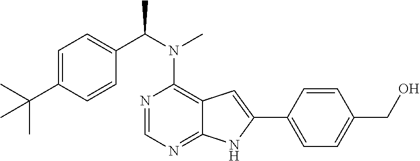 [SMILES: C[C@H](c1ccc(C(C)(C)C)cc1)N(C)c1ncnc2[nH]c(-c3ccc(CO)cc3)cc12]

¹H NMR (400 MHz, DMSO-d₆) δ: 12.20 (br s, 1H), 8.17 (s, 1H), 7.90-7.85 (m, 2H), 7.44-7.38 (m, 2H), 7.37-7.32 (m, 4H), 7.30-7.24 (m, 2H), 7.11 (s, 1H), 6.50-6.42 (m, 1H), 5.19 (t, J=5.7 Hz, 1H), 4.51 (d, J=5.7 Hz, 2H), 3.08 (s, 3H), 1.60 (d, J=7.0 Hz, 3H), 1.25 (s, 9H); ¹³C NMR (100 MHz, DMSO-d₆) δ: 156.6, 153.1, 151.1, 149.4, 141.6, 138.5, 133.1, 130.0, 127.0 (2C), 126.8 (2C), 125.3 (2C), 124.5 (2C), 103.5, 99.1, 62.6, 52.1, 34.5, 31.6, 31.5 (3C), 16.2; HRMS (ASAP+, m/z): found 415.2487, calcd for C₂₆H₃₁N₄O, [M+H]⁺, 415.2492.

### (4-(4-(Benzyl(isopropyl)amino)-7H-pyrrolo[2,3-d]pyrimidin-6-yl)phenyl) methanol (TIA02-054)

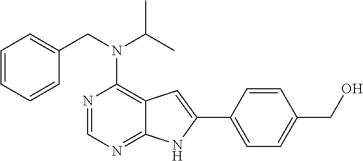 [SMILES: CC(C)N(Cc1ccccc1)c1ncnc2[nH]c(-c3ccc(CO)cc3)cc12]

¹H NMR (400 MHz, DMSO-d₆) δ: 12.10 (s, 1H), 8.13 (s, 1H), 7.72-7.65 (m, 2H), 7.35-7.30 (m, 2H), 7.30-7.27 (m, 4H), 7.24-7.14 (m, 1H), 6.70 (s, 1H), 5.27-5.19 (m, 1H), 5.18 (t, J=5.7 Hz, 1H), 4.97 (s, 2H), 4.49 (d, J=5.7 Hz, 2H), 1.21 (d, J=6.7 Hz, 6H); HRMS (ASAP+, m/z): found 373.2022, calcd for C₂₃H₂₅N₄O, [M+H]⁺, 373.2028.

### N-(4-Methoxybenzyl)-N-methyl-8-(4-(hydroxymethyl)phenyl)-9H-purin-6-amine (TIA03-078)

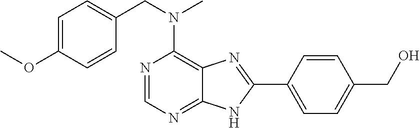 [SMILES: COc1ccc(CN(C)c2ncnc3[nH]c(-c4ccc(CO)cc4)nc23)cc1]

¹H NMR (600 MHz, DMSO-d₆) δ: 13.43 (s, 1H), 8.23 (s, 1H), 8.10-8.06 (m, 2H), 7.47-7.42 (m, 2H), 7.29-7.24 (m, 2H), 6.91-6.86 (m, 2H), 5.46 (s, 2H), 5.28 (t, J=5.7 Hz, 1H), 4.55 (d, J=5.6 Hz, 2H), 3.71 (s, 3H), 3.26 (s, 3H); ¹³C NMR (151 MHz, DMSO-d₆) δ: 158.4, 153.5, 152.8, 151.9, 147.1, 144.5, 130.1, 128.9 (2C), 128.0, 126.8 (2C), 125.9 (2C), 119.8, 113.9 (2C), 62.5, 55.0; HRMS (ASAP+, m/z): found 376.1766, calcd for C₂₁H₂₂N₅O₂, [M+H]⁺, 376.1773.

### N-Benzyl-8-(2-ethoxyphenyl)-N-methyl-9H-purin-6-amine (JKS01-057)

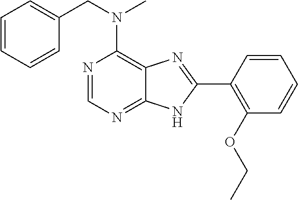 [SMILES: CCOc1ccccc1-c1nc2c(N(C)Cc3ccccc3)ncnc2[nH]1]

¹H NMR (600 MHz, CDCl₃) δ: 10.76 (s, 1H), 8.42 (d, J=7.7 Hz, 1H), 8.36 (s, 1H), 7.39-7.35 (m, 3H), 7.33-7.31 (m, 1H), 7.27-7.25 (m, 2H), 7.07 (t, J=7.7 Hz, 1H), 7.04-7.03 (m, 1H), 5.50 (s br, 2H), 4.30 (q, J=7.0 Hz, 2H), 3.46 (br s., 3H), 1.61 (t, J=7.0 Hz, 3H); ¹³C NMR (150 MHz, CDCl₃) δ: 156.0, 154.3, 152.2, 151.2, 145.0, 138.2, 131.1, 129.4, 128.5, 127.8 (2C), 127.2 (2C), 121.4, 119.7, 117.6, 112.3, 64.6, 54.0, 35.8, 14.9; HRMS (APCI/ASAP+, m/z) found 360.1818, calcd. C₂₁H₂₂N₅O, [M+H]⁺, 360.1824.

### N-Benzyl-N-ethyl-8-(4-methoxyphenyl)-9H-purin-6-amine (EH-93)

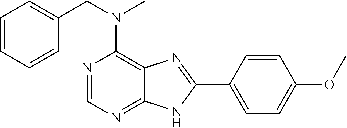 [SMILES: COc1ccc(-c2nc3c(N(C)Cc4ccccc4)ncnc3[nH]2)cc1]

¹H NMR (400 MHz, DMSO-d₆) δ: 13.35 (br s, 1H), 8.20 (s, 1H), 8.05 (d, J=8.5 Hz, 2H), 7.34-7.22 (m, 5H), 7.07 (d, J=8.8 Hz, 2H), 5.21 (br s, 2H), 3.94 (br s, 2H), 3.81 (s, 3H), 1.19 (s, 3H); ¹³C NMR (100 MHz, DMSO-d₆) δ: 160.6, 152.9, 151.6, 147.3, 139.0, 128.4 (2C), 127.7 (2C), 127.4 (2C), 126.9, 122.2, 119.5, 114.3 (2C), 55.3, 50.1, 42.4, 12.9; HRMS (APCI/ASAP+, m/z): found 360.1818, calcd for C₂₁H₂₂N₅O, [M+H]⁺, 360.1824, mp. 197-198° C.

### (4-(6-(Benzyl(isopropyl)amino)-9H-purin-8-yl)phenyl)methanol (EH02-21)

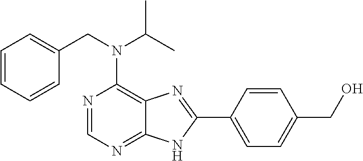 [SMILES: CC(C)N(Cc1ccccc1)c1ncnc2[nH]c(-c3ccc(CO)cc3)nc12]

¹H NMR (600 MHz, DMSO-d₆) δ: 13.44 (s, 1H), 8.18 (s, 1H), 8.04 (br s, 2H), 7.44 (d, J=6.7 Hz, 2H), 7.27-7.16 (m, 5H), 6.24 (br s, 1H), 5.28 (t, J=5.3 Hz, 1H), 5.07 (br s, 2H), 4.55 (d, J=4.9 Hz, 2H), 1.21 (d, J=5.8 Hz, 6H); ¹³C NMR (150 MHz, DMSO-d₆) δ: 153.6, 153.0, 151.8, 147.1, 144.5, 140.8, 128.0 (2C), 126.7 (2C), 126.5 (2C), 126.1, 125.9 (2C), 119.8, 62.5, 48.1, 44.6, 20.6; HRMS (APCI/ASAP+, m/z), found 374.1977, calcd for C₂₂H₂₄N₅O, [M+H]⁺, 374.1981; mp. 229-231° C.

### N-Methyl-N-(1-benzylpiperidin-4-ylmethyl)-8-(4-(hydroxymethyl)phenyl)-9H-purin-6-amine (TIA04-053)

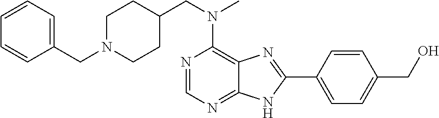 [SMILES: CN(CC1CCN(Cc2ccccc2)CC1)c1ncnc2[nH]c(-c3ccc(CO)cc3)nc12]

¹H NMR (600 MHz, DMSO-d₆) δ: 13.38 (s, 1H), 8.18 (s, 1H), 8.11-8.07 (m, 2H), 7.48-7.44 (m, 2H), 7.31-7.23 (m, 4H), 7.23-7.19 (m, 1H), 5.29 (t, J=5.7 Hz, 1H), 4.56 (d, J=5.5 Hz, 2H), 3.40 (s, 2H), 2.81-2.75 (m, 2H), 1.92-1.82 (m, 3H), 1.64-1.58 (m, 2H), 1.36-1.27 (m, 2H). Missing signals: N(CH₃)CH₂; ¹³C NMR (151 MHz, DMSO-d₆) δ: 153.7, 152.6, 151.8, 146.7, 144.4, 138.7, 128.6 (2C), 128.11, 128.06 (2C), 126.8 (2C), 126.7, 125.8 (2C), 119.8, 62.5, 62.4, 52.9 (2C), 29.5 (2C); HRMS (ASAP+, m/z): found 443.2556, calcd for C₂₆H₃₁N₆O, [M+H]⁺, 443.2559.

### N-Methyl-N-(1-methylpiperidin-3-ylmethyl)-8-(4-(hydroxymethyl)phenyl)-9H-purin-6-amine (TIA04-063)

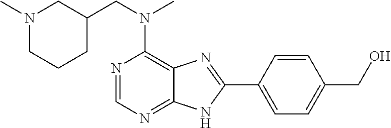 [SMILES: CN1CCCC(CN(C)c2ncnc3[nH]c(-c4ccc(CO)cc4)nc23)C1]

¹H NMR (600 MHz, DMSO-d₆) δ: 13.38 (s, 1H), 8.19 (s, 1H), 8.11-8.06 (m, 2H), 7.48-7.44 (m, 2H), 5.29 (s, 1H), 4.56 (s, 2H), 2.69-2.66 (m, 1H), 2.63-2.57 (m, 1H), 2.15-2.10 (m, 1H), 2.11 (s, 3H), 1.86-1.83 (m, 1H), 1.78-1.75 (m, 1H), 1.68-1.59 (m, 2H), 1.46-1.39 (m, 1H), 1.04-0.99 (m, 1H). Missing signals: NCH₂, NCH₃; ¹³C NMR (151 MHz, DMSO-d₆) δ: 153.7, 152.6, 151.8, 146.8, 144.4, 128.1, 126.8 (2C), 125.8 (2C), 119.8, 62.5, 59.4, 55.8, 53.7, 46.5, 36.9, 35.6, 27.4, 24.4; HRMS (ASAP+, m/z): found 367.2251, calcd for C₂₀H₂₇N₆O, [M+H]⁺, 367.2251.

### (4-(4-(Benzyl(methyl)amino)thieno[2,3-d]pyrimidin-6-yl)phenyl)methanol (BAHO-3-107)

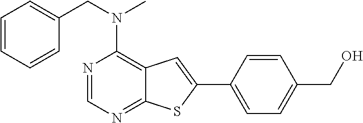 [SMILES: CN(Cc1ccccc1)c1ncnc2sc(-c3ccc(CO)cc3)cc12]

¹H NMR (400 MHz, DMSO-d₆) δ: 8.36 (s, 1H), 7.86 (s, 1H), 7.72-7.65 (m, 2H), 7.42-7.23 (m, 7H), 5.25 (t, J=5.7 Hz, 1H), 5.10 (s, 2H), 4.52 (d, J=5.7 Hz, 2H), 3.47 (s, 3H); ¹³C NMR (101 MHz, DMSO-d₆) δ: 167.9, 157.4, 152.7, 143.0, 137.7, 136.4, 131.5, 128.6 (2C), 127.1 (3C), 126.9 (2C), 125.6 (2C), 117.6, 116.4, 62.5, 53.7, 39.8; HRMS (TOF/ASAP, m/z): found 362.1332, calcd C₂₁H₂₀N₃OS, [M+H]⁺, 362.1327.

### Testing Methods for CSF-1R Inhibition

### CSF1R Enzymatic Inhibitory Assay

The compounds were supplied in a 10 mM DMSO solution, and enzymatic CSF1R inhibition potency was determined by Invitrogen (TermoFisher) using their Z′-LYTE® assay technology (B. A. Pollok, B. D. Hamman, S. M. Rodems, L. R. Makings, Optical probes and assays, WO 2000066766 A1, 5-5-2000.). The assay is based on fluorescence resonance energy transfer (FRET). In the primary reaction, the kinase transfers the gamma-phosphate of ATP to a single tyrosine residue in a synthetic FRET-peptide. In the secondary reaction, a site-specific protease recognizes and cleaves non-phosphorylated FRET-peptides. Thus, phosphorylation of FRET-peptides suppresses cleavage by the development reagent. Cleavage disrupts FRET between the donor (i.e.,coumarin) and acceptor (i.e., fluorescein) fluorophores on the FRET-peptide, whereas uncleaved, phosphorylated FRET-peptides maintain FRET. A ratiometric method, which calculates the ratio (the emission ratio) of donor emission to acceptor emission after excitation of the donor fluorophore at 400 nm, is used to quantitate inhibition. All compounds were first tested for their inhibitory activity at 500 nM in duplicates. The potency observed at 500 nM was used to set starting point of the IC₅₀ titration curve, in which three levels were used 1000 or 10000 nM. The IC₅₀ values reported are based on the average of at least 2 titration curves (minimum 20 data points), and were calculated from activity data with a four parameter logistic model using SigmaPlot (Windows Version 12.0 from Systat Software, Inc.) Unless stated otherwise the ATP concentration used was equal to Kₘ (ca 10 mM). The average standard deviation for single point measurements were <4%.

### Effect of CSF1R Inhibitors on MAPK Signaling in Mouse Bone-Marrow Derived Macrophages

Bone-marrow derived macrophages were obtained by flushing the femur and tibia of sacrificed mice with HBSS (Hanks' balanced salt solution) using a syringe with a 25G needle. The cells were centrifuged at 1500 rpm for 8 minutes, the resulting supernatant was decanted, and the cells resuspended in 5 mL RBC (red blood cell) lysis buffer. Lysis was stopped by adding 30 ml medium containing 10% FCS (Fetal Calf Serum). The cells were centrifuged at 1500 rpm for 8 minutes. The supernatant was decanted, and the cells resuspended in RPMI medium gentamycin, 2 mM glutamine, 10% FCS) with 10 ng/mL CSF-1 (colony stimulating factor 1). The cells were seeded in bacteria plates. After two days, fresh medium with 10 ng/mL CSF-1 was added and after another two days, 50% of the medium was replaced with fresh medium containing 10 ng/mL CSF-1 while the other 50% is centrifuged to get rid of dead cells before being transferred back to the cells. After incubating for one week, the differentiated cells were washed twice with PBS, added PBS EDTA (0.2 mM) and incubated for 10 minutes. Cells were detached by scraping and centrifuged at 1200 rpm for 7 minutes. The supernatant was decanted, and the cells resuspended in RPMI medium (10% FCS, 10 ng/ml CSF-1, x gentamycin, 2 mM L-glutamine). The cells were seeded out in 96-well glass bottom plates (Cellvis) at 50.000 cells in 100 μL per well and incubated at 37° C. overnight. The medium was removed, and the cells washed three times with PBS before being starved overnight in 0.1% FCS medium without CSF-1. CSF-1 inhibitors dissolved in DMSO, was added to the wells in appropriate concentrations and incubated for 30 minutes at 37° C. DMSO was added to control wells at the highest inhibitor concentration. CSF-1 (0.1 mg/mL) was added to all wells except the CSF-1 negative control to obtain an end concentration of CSF-1 of 10 ng/mL. After incubating for another 10 minutes at 37° C., the cells were fixed by adding PFA (paraformaldehyde) (16%) to obtain an end concentration of 4% for 10 minutes. The cells was washed twice with TBS, and permabilized by MeOH for 10 minutes on ice. The cells were washed twice with TBS and blocked in Odyssey blocking solution (Licor, #927-60001) diluted 1:1 in TBS-Tween (0.1%) for 1.5 hours under careful agitation. The blocking solution was removed and the wells added appropriately diluted primary antibody solution (P-MAPK (ERK1/2, Thr202/Tyr204) (CST, #4370, rabbit) 1:1200 and MAPK (ERK1/2), (Biolegend, #686902, rat) 1:300 in in Odyssey blocking buffer:TBS-Tween (1:1)). After incubating overnight with careful shaking at 4° C., the cells were washed with TBST 5 times for 5 minutes while agitating. Secondary antibody solution was added, and the wells were incubated for 1 hour in the dark Rdye 800CW goat anti-rabbit and IRdye 680RD goat anti-rat diluted 1:800 in Odyssey blocking buffer:TBS-Tween (1:1)). The antibody solution was removed, and the wells were washed 4×5 minutes with TBST and 2×5 minutes with TBS while carefully agitating. The TBS was removed and the plate was scanned on a Licor Odyssey scanner. Using Image Studio software, the intensity of fluorescence of each well is recorded after subtracting the background noise (wells not added primary antibody). The results were normalized by dividing the P-MAPK intensity with total MAPK intensity for all the wells. The average value of triplicate wells was calculated for every concentration of inhibitor used. The average values are then divided by the CSF-1 positive control value. PLX3397 was included as a reference on all plates. Due to some inter-assay variation the activity of the inhibitors is reported as fold change relative to PLX3397 (Fold change: IC₅₀.inhibitor/IC₅₀ PLX3397)

The CSF-1R inhibition results are given below. The compounds shown in the tables below have been synthesised and tested.

TABLE 10
Pyrrolopyrimidines (variation at C-6)

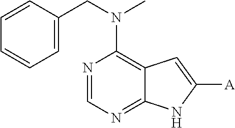

| | | CSF1R | |
|---|---|---|---|
| | | (IC₅₀, | MAPK |
| Comp. | A-group/compound | nM) | (fold) |
| KUL01-097 | 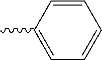 | 15 ± 0 | 1.7 |
| TIA04-067 (comparative) | 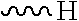 | 68 ± 2 | ND |
| KUL01-123 | 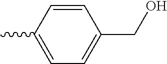 | 1.0 ± 0.9* | 1.0 |
| TIA085 (comparative) | 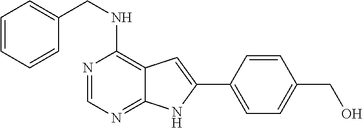 [SMILES: OCc1ccc(-c2cc3c(NCc4ccccc4)ncnc3[nH]2)cc1] | 8.3 ± 0.2 | ND |
| BAHO-3-107 (comparative) | 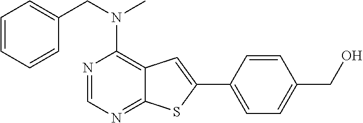 [SMILES: CN(Cc1ccccc1)c1ncnc2sc(-c3ccc(CO)cc3)cc12] | 707 ± 79 | ND |
| TIA04-181 | 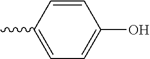 | 2.6 ± 0.7 | 2.1 |
| JK-367 (comparative) | 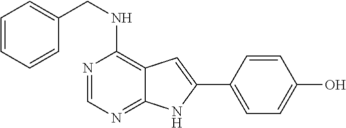 [SMILES: Oc1ccc(-c2cc3c(NCc4ccccc4)ncnc3[nH]2)cc1] | >200 | ND |
| KUL02-024 | 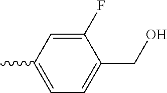 | 3.7 ± 0.3 | >5 |
| KUL02-016 | 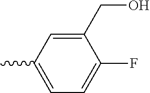 | 2.1 ± 0.3 | >5 |
| TL1-62 | 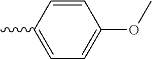 | 1.3 ± 0.2 | 1.5 |
| JK-356 (comparative) | 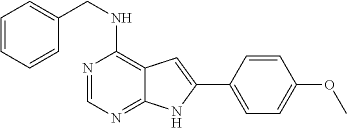 [SMILES: COc1ccc(-c2cc3c(NCc4ccccc4)ncnc3[nH]2)cc1] | 6.2 ± 0.4 | ND |
| TL1-86 | 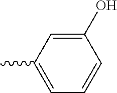 | 2.8 ± 0.1 | >3 |
| TL1-78 | 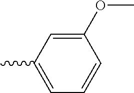 | 5.2 ± 0.1 | ND |
| TL1-122 | 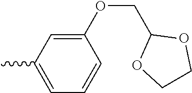 | 3.7 ± 0.3 | ND |
| TL1-70 | 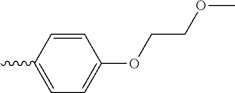 | 2.0 ± 0.1 | 1.4 |
| TIA03-124 | 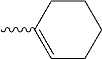 | 1.0 ± 0.2 | ND |
| NOR-4-001 | 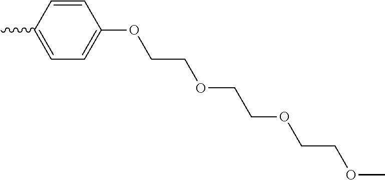 | 0.8 ± 0.0 | 1.2 |
| KUL02-056 | 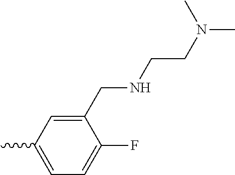 | 0.8 ± 0.1 | >3 |
| KUL02-028 | 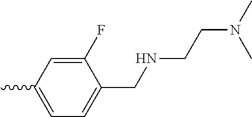 | 0.4 ± 0.1* | >2 |
| PLX-3397 (Positive control) | 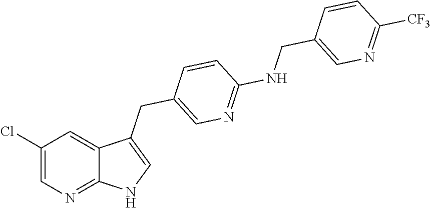 [SMILES: FC(F)(F)c1ccc(CNc2ccc(Cc3c[nH]c4ncc(Cl)cc34)cn2)cn1] | 9.7 ± 3.9* | 1 |
| PLX5622 | 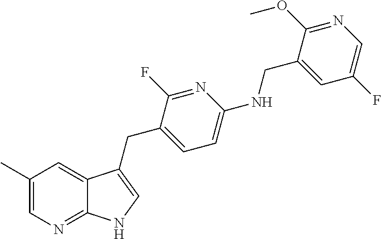 [SMILES: COc1ncc(F)cc1CNc1ccc(Cc2c[nH]c3ncc(C)cc23)c(F)n1] | 19 ± 1 | 1.6 |
| GW-2580 (Positive control) | 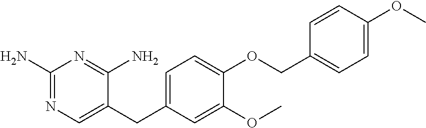 [SMILES: COc1ccc(COc2ccc(Cc3cnc(N)nc3N)cc2OC)cc1] | 6.4 ± 0.3 | ND |

TABLE 11
Pyrrolopyrimidines (variation at C-6)

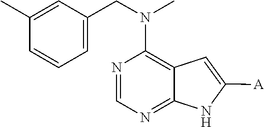

| | | CSFIR (IC₅₀, | MAPK |
|---|---|---|---|
| Comp. | A-group | nM) | (fold) |
| TIA04-069 (comparative) | 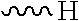 | 35 ± 0 | ND |
| TIA02-056 | 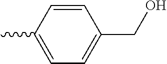 | 0.5 ± 0.2* | 0.8 |
| FAB01-35 | 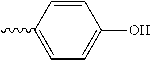 | 1.4 ± 0.1 | 1.2 |
| FAB01-39/ FA01-72 | 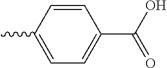 | 0.3 ± 0.0* | 2.5 |
| TIA03-104 | 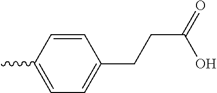 | 0.2 ± 0.1 | 1.9 |
| FAB01-45 | 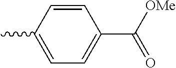 | 4.0 ± 0.2 | 1.3 |
| FAB 01-62 | 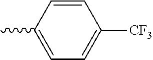 | 4.9 ± 0.2 | ND |
| FAB 01-69 | 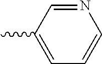 | 0.7 ± 0.0 | 5.5 |
| FAB 01-63 | 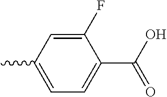 | 0.4 ± 0.0 | ND |
| FAB 01-61 | 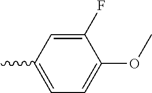 | 1.4 ± 0.0 | ND |
| FAB 01-93 | 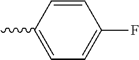 | 2.7 ± 0.0 | ND |
| FAB 01-92 | 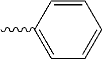 | 1.8 ± 0.0 | ND |
| LR-1-107 | 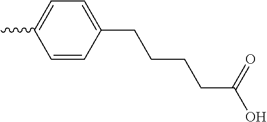 | 0.5 ± 0.3 | 1.6 |
| LR-1-119 | 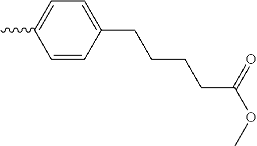 | 10 ± 0 | 0.7 |
| LR-1-124 | 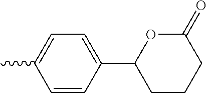 | 0.8 ± 0.1 | 2.1 |
| LR-1-138 | 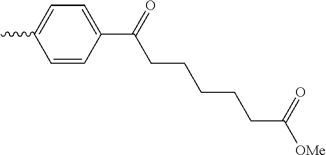 | 16 ± 1 | ND |
| NOR-4-004 | 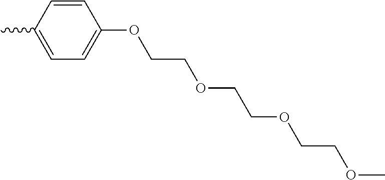 | 0.4 ± 0.3 | 2.0 |
| FAB02-15 | 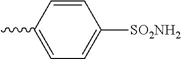 | 0.4 ± 0.0 | 3.1 |
| FAB02-13 | 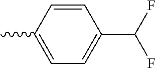 | 3.0 ± 0.3 | ND |
| FAB01-23 | 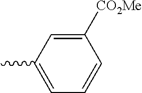 | 6.4 ± 0.5 | ND |
| FAB01-25 | 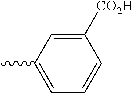 | 0.6 ± 0.1 | ND |
| LR-2-060-1 | 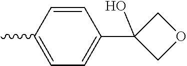 | 0.3 ± 0.0 | ND |
| LR-2-060-2 | 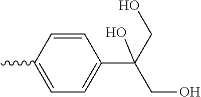 | 3 | ND |
| LR-2-116 | 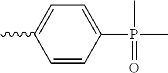 | 0.2< | ND |
| LR-2-108 | 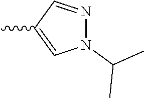 | 0.4 ± 0.3 | ND |
| FAB 02-14 | 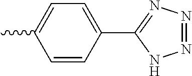 | 3 | ND |

TABLE 12
Pyrrolopyrimidines (variation at C-6)

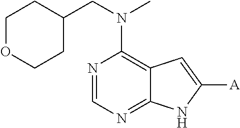

| | | CSFIR (IC₅₀, | |
|---|---|---|---|
| Comp. | A-group | nM) | MAPK (fold) |
| SH-01-18 | 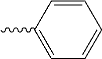 | 0.6 ± 0.0 | >3.5 |
| TIA05-028 | 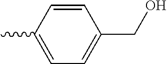 | 0.5 ± 0.1 | >3 |
| TIA05-032 | 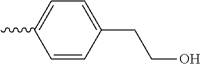 | <0.2 | 2.3 |
| SH-01-17 | 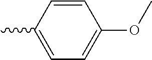 | 0.3 ± 0.0 | >3.5 |
| SH-01-26 | 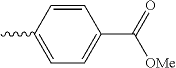 | 0.9 ± 0.1 | ND |
| TIA05-030 | 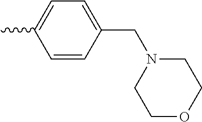 | 0.2 ± 0.0 | 1.3 |
| SH-01-27 | 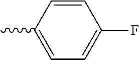 | 0.8 ± 0.0 | >3.5 |
| SH-01-45-P1 | 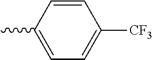 | 1.9 ± 0.2 | ND |
| SH-01-58 | 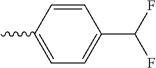 | 0.8 ± 0.2 | >3.5 |
| SH-01-30-P1 | 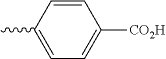 | 0.4 ± 0.0 | >3.5 |
| SH-01-59 | 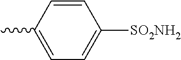 | 0.2 ± 0.0 | >3.5 |
| SH-01-39-P1 | 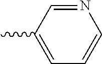 | 1.4 ± 0.1 | >3.5 |
| SH-01-44-P1 | 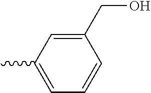 | 0.6 ± 0.0 | ND |
| SH-01-43-P1 | 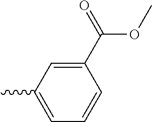 | 2.2 ± 0.5 | ND |
| SH-01-50 | 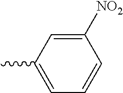 | 2.7 ± 0.0 | ND |
| SH-01-53 | 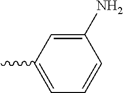 | 0.4 ± 0.0 | ND |
| FAB 02-55 | 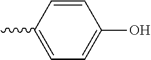 | 0.3 ± 0.1 | ND |
| FAB 02-61 | 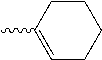 | 0.4 ± 0.0 | ND |
| SH-01-76 | 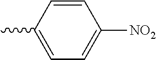 | 0.8 ± 0.1 | ND |
| SH-01-82 | 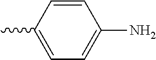 | 0.3 ± 0.0 | ND |
| SH-01-92 | 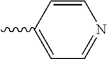 | 1.1 ± 0.2 | ND |
| TIA06-003 | 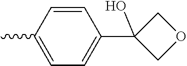 | <0.2 | ND |
| FAB 04-09 | 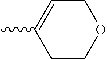 | 0.4 ± 0.0 | ND |

TABLE 13
Pyrrolopyrimidines (variation at C-4)

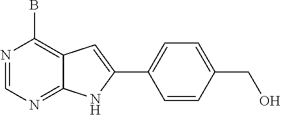

| | | CSF1R | |
|---|---|---|---|
| Comp. | Amine (B-group) | (IC₅₀, nM) | MAPK (fold) |
| TIA02-072 | 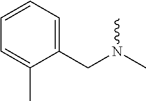 | 0.7 ± 0.1 | >3 |
| TIA02-074 | 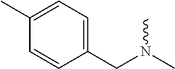 | 2.4 ± 0.0 | ND |
| TIA03-096 | 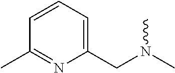 | 0.5 ± 0.2 | >3 |
| TIA02-076 | 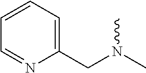 | 1.1 ± 0.2 | >3 |
| TIA086 | 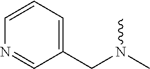 | 2.5 ± 0.5 | 3.0 |
| TIA02-052 | 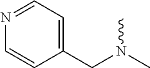 | 1.0 ± 0.1 | 1.7 |
| TIA03-126 | 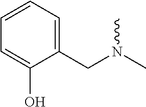 | 0.3 ± 0.0 | 1.1 |
| TIA04-139 | 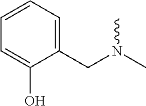 | 1.7 ± 0.1 | >3 |
| TIA05-086 | 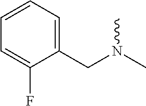 | 0.3 ± 0.1 | 1.5 |
| KUL01-123 | 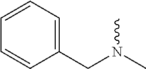 | 1.0 ± 0.9 | 1.0 |
| TIA084 | 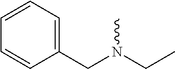 | 6.2 ± 0.3 | ND |
| TIA-085 (comparative) | 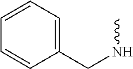 | 8.3 ± 0.2 | ND |
| TIA05-106 (comparative) | 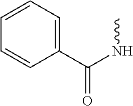 | 180 | ND |
| TIA05-088-2 (comparative) | 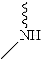 | 11.5 | ND |
| TIA05-102 (comparative) | 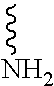 | 85 | ND |
| TIA02-054 (comparative) | 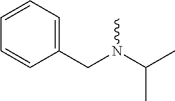 | >100 | ND |
| TIA05-046 | 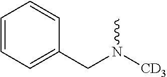 | 0.2 ± 0.1 | 0.6 |
| TIA05-088 | 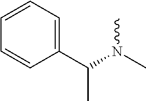 | 1.7 ± 0.4 | >5 |
| TIA097 | 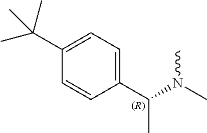 | 55 ± 4 | ND |
| TIA05-010 | 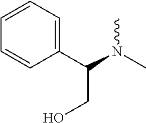 | 127 ± 23 | >3 |
| JH06-118 (comparative) | 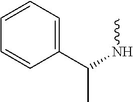 | 12 ± 1 | ND |
| TIA05-008 | 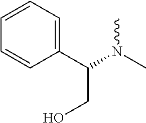 | 2.8 ± 0.4 | >3 |
| TIA05-090 | 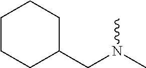 | 0.3 ± 0.0 | 1.0 |
| BAHO-3-107 (comparative) | 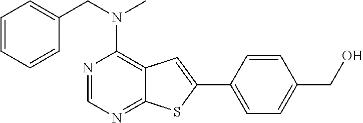 [SMILES: CN(Cc1ccccc1)c1ncnc2sc(-c3ccc(CO)cc3)cc12] | 707 ± 79 | ND |

TABLE 14
Further suitable pyrrolopyrimidines:

| | | CSF1R | MAPK |
|---|---|---|---|
| Comp. | Structure | (IC₅₀, nM) | (fold) |
| HHMT1- 170 | 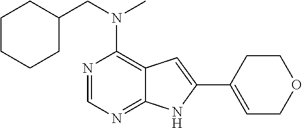 [SMILES: CN(CC1CCCCC1)c1ncnc2[nH]c(C3=CCOCC3)cc12] | <0.2 | 0.6 |
| HHMT-070 | 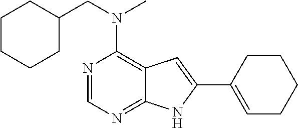 [SMILES: CN(CC1CCCCC1)c1ncnc2[nH]c(C3=CCCCC3)cc12] | ND | ND |
| FAB01- 23 | 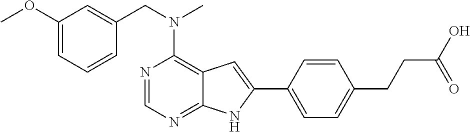 [SMILES: COc1cccc(CN(C)c2ncnc3[nH]c(-c4ccc(CCC(=O)O)cc4)cc23)c1] | 0.2 ± 0.0 | >5 |
| FAB 01- 24 | 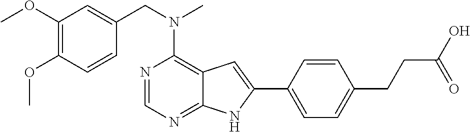 [SMILES: COc1ccc(CN(C)c2ncnc3[nH]c(-c4ccc(CCC(=O)O)cc4)cc23)cc1OC] | 0.6 ± 0.0 | >5 |
| TIA05- 178 | 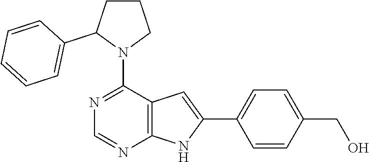 [SMILES: OCc1ccc(-c2cc3c(N4CCCC4c4ccccc4)ncnc3[nH]2)cc1] | 0.7 ± 0.1* | ND |
| MS1-60- 134 | 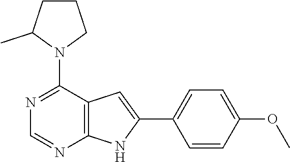 | 0.7 ± 0.1 | ND |
| MS-46-93 | 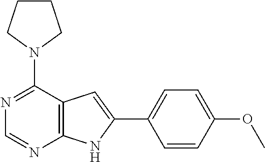 [SMILES: COc1ccc(-c2cc3c(N4CCCC4)ncnc3[nH]2)cc1] | 1.1 ± 0.1 | ND |
| KUL01- 093 | 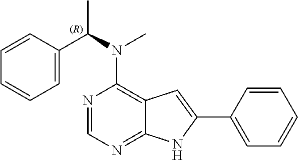 | 54 ± 5 | ND |
| SH-01- 112 | 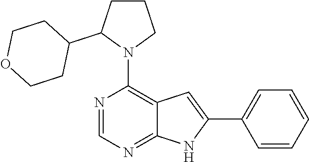 [SMILES: c1ccc(-c2cc3c(N4CCCC4C4CCOCC4)ncnc3[nH]2)cc1] | 2.6 ± 1.0 | ND |
| SH-01- 118 | 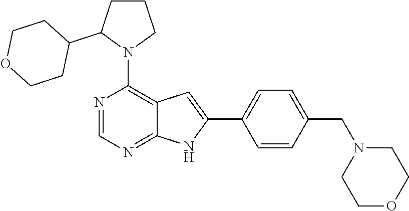 [SMILES: c1nc(N2CCCC2C2CCOCC2)c2cc(-c3ccc(CN4CCOCC4)cc3)[nH]c2n1] | 2.2 ± 0.3 | ND |
| SH-01- 126 | 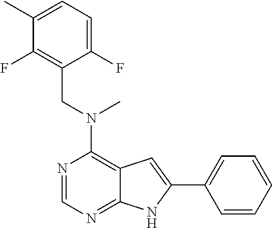 [SMILES: Cc1ccc(F)c(CN(C)c2ncnc3[nH]c(-c4ccccc4)cc23)c1F] | 9.1 ± 1.1 | ND |
| SH-01- 127 | 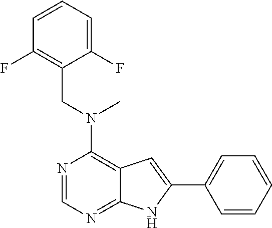 [SMILES: CN(Cc1c(F)cccc1F)c1ncnc2[nH]c(-c3ccccc3)cc12] | 9.2 ± 3.3 | ND |
| SH-01- 128 | 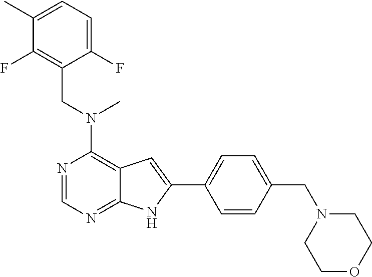 [SMILES: Cc1ccc(F)c(CN(C)c2ncnc3[nH]c(-c4ccc(CN5CCOCC5)cc4)cc23)c1F] | 0.8 | ND |
| SH-01- 129 | 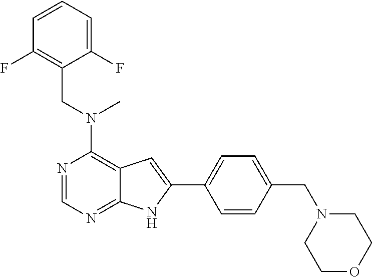 [SMILES: CN(Cc1c(F)cccc1F)c1ncnc2[nH]c(-c3ccc(CN4CCOCC4)cc3)cc12] | 1.2 ± 0.0 | ND |
| SH-01-99 | 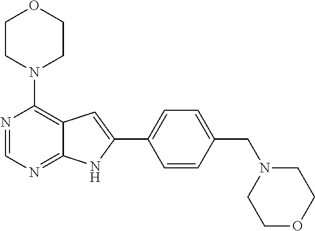 [SMILES: c1nc(N2CCOCC2)c2cc(-c3ccc(CN4CCOCC4)cc3)[nH]c2n1] | 3 ± 0.1 | ND |
| LR-2-067 | 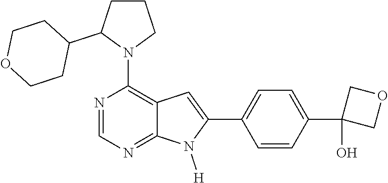 [SMILES: OC1(c2ccc(-c3cc4c(N5CCCC5C5CCOCC5)ncnc4[nH]3)cc2)COC1] | 3 | ND |
| LR-2- 052-2 | 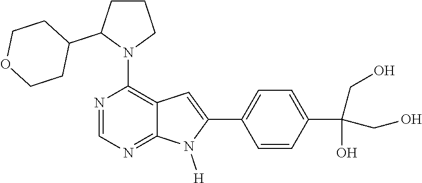 [SMILES: OCC(O)(CO)c1ccc(-c2cc3c(N4CCCC4C4CCOCC4)ncnc3[nH]2)cc1] | 3 | ND |
| LR-2-088 (3) | 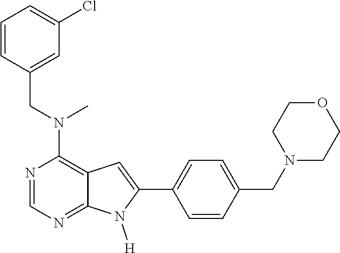 [SMILES: CN(Cc1cccc(Cl)c1)c1ncnc2[nH]c(-c3ccc(CN4CCOCC4)cc3)cc12] | 0.9 ± 0.9 | ND |
| LR-2-091 (2) | 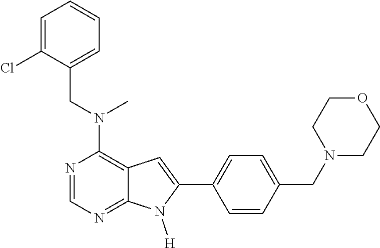 [SMILES: CN(Cc1ccccc1Cl)c1ncnc2[nH]c(-c3ccc(CN4CCOCC4)cc3)cc12] | 1.4 ± 0.2 | ND |
| LR-2-094 (2) | 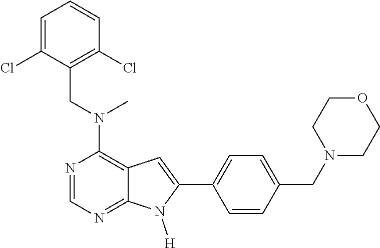 [SMILES: CN(Cc1c(Cl)cccc1Cl)c1ncnc2[nH]c(-c3ccc(CN4CCOCC4)cc3)cc12] | 8.7 ± 1.1 | ND |
| LR-2-123 | 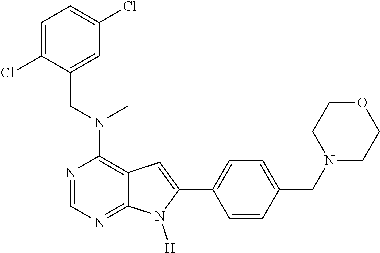 [SMILES: CN(Cc1cc(Cl)ccc1Cl)c1ncnc2[nH]c(-c3ccc(CN4CCOCC4)cc3)cc12] | 2.7 ± 0.4 | ND |
| FAB 04- 15 | 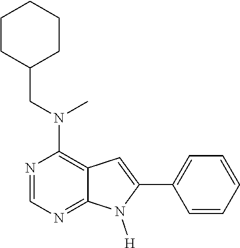 [SMILES: CN(CC1CCCCC1)c1ncnc2[nH]c(-c3ccccc3)cc12] | 1.5 ± 0.3 | ND |

TABLE 15
Purines

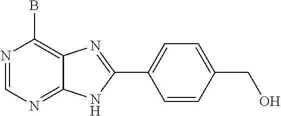

| | | CSF1R (IC₅₀, | |
|---|---|---|---|
| Comp. | B-group (Amine) | nM) | MAPK (fold) |
| TIA02-130 | 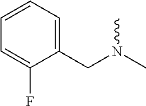 | 2.3 ± 0.2 | ND |
| TIA03-136 | 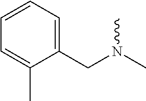 | 1.8 ± 0.0 | ND |
| TIA03-106 | 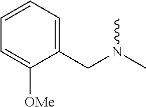 | 4.2 ± 1.4 | >3 |
| TIA02-164 | 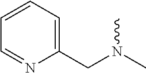 | 11 ± 3 | ND |
| TIA070 | 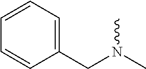 | 1.1 ± 0.1 | 2.5 |
| EH02-21 (comparative) | 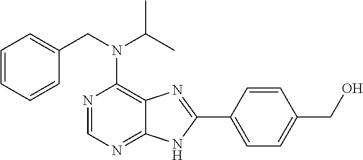 [SMILES: CC(C)N(Cc1ccccc1)c1ncnc2[nH]c(-c3ccc(CO)cc3)nc12] | >1000 | ND |
| TIA098 (comparative) | 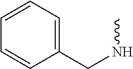 | 43 ± 6 | ND |
| TIA02-132 | 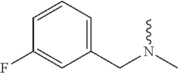 | 3.5 ± 2.2 | ND |
| TIA02-172 | 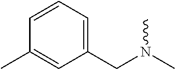 | 1.5 ± 0.2 | >5 |
| TIA03-014 | 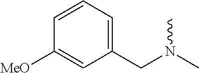 | 2.7 ± 0.1 | ND |
| TIA03-016 | 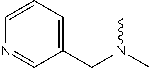 | 3.8 ± 0.4 | ND |
| TIA04-051 | 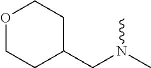 | 0.3 ± 0.0 | 1 |
| TIA02-134 | 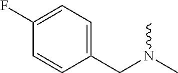 | 8.8 ± 0.3 | >3 |
| TIA02-174 | 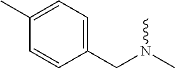 | 12 ± 1 | ND |
| TIA03-078 | 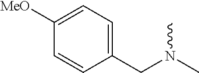 | 61 ± 3 | ND |
| TIA02-166 | 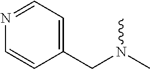 | 2.1 ± 0.4 | >3 |
| TIA04-063 | 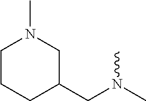 | 53 ± 4 | >3 |
| TIA04-053 (comparative) | 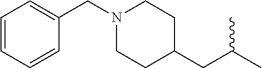 | 130 ± 12 | ND |
| TIA02-176 | 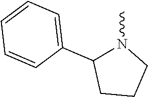 | 1.9 ± 0.2 | >3 |
| TIA03-024 | 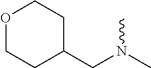 | 0.7 ± 0.0 | 0.9 |
| TIA03-066 | 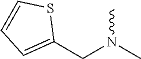 | 3.0 ± 0.6 | ND |
| TIA04-095 | 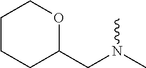 | 1.3 ± 0.2 | >3 |
| TIA04-009 | 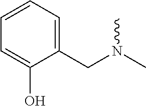 | 2.3 ± 0.3 | >3 |
| TIA03-108 | 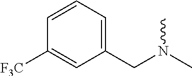 | 11 ± 2 | ND |
| TIA03-080 | 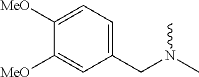 | 5.7 ± 0.3 | ND |
| TIA03-188 | 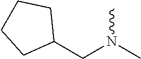 | 0.3 ± 0.2 | 0.6 |
| TIA04-093 | 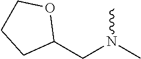 | 2.6 ± 0.3 | >3 |
| TIA04-049 | 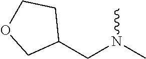 | 4.6 ± 0.6 | >3 |
| TIA04-065 | 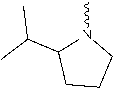 | 5.4 ± 2.4 | >3 |

TABLE 16
Purines

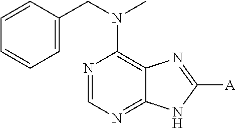

| | | CSF1R | MAPK |
|---|---|---|---|
| Comp. | A-group/compounds | (IC_{50•} nM) | (fold) |
| TIA072 | 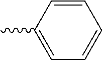 | 0.7 ± 0.1 | ND |
| TIA008 (comparative) | 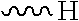 | 108 ± 7 | ND |
| TIA099 (comparative) | 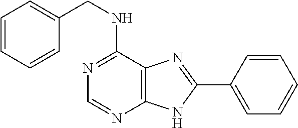 [SMILES: c1ccc(CNc2ncnc3[nH]c(-c4ccccc4)nc23)cc1] | 85 ± 10 | ND |
| TIA073 | 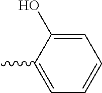 | 3.4 ± 0.2 | ND |
| JKS02-027 | 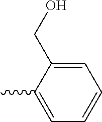 | 1.2 ± 0.2 | >3 |
| TIA04-105 | 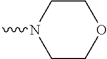 | 16 ± 4 | >3 |
| JKS01-057 | 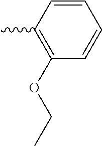 | >1000 | ND |

TABLE 17
Purines

| | | CSF1R (IC_{50•} | |
|---|---|---|---|
| Comp. | Compounds | nM) | MAPK (fold) |
| TIA04-117 | 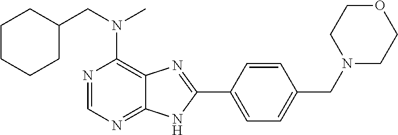 [SMILES: CN(CC1CCCCC1)c1ncnc2[nH]c(-c3ccc(CN4CCOCC4)cc3)nc12] | 1.2 ± 0.2 | 1.2 |
| TIA03-186 | 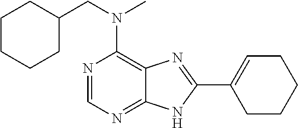 [SMILES: CN(CC1CCCCC1)c1ncnc2[nH]c(C3=CCCCC3)nc12] | 0.7 ± 0.2 | ND |
| TIA05-014 | 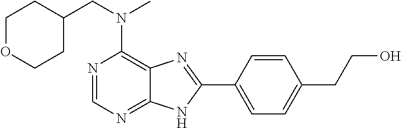 [SMILES: CN(CC1CCOCC1)c1ncnc2[nH]c(-c3ccc(CCO)cc3)nc12] | 0.2 ± 0.0 | 1.7 |
| TIA109 | 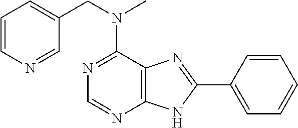 [SMILES: CN(Cc1cccnc1)c1ncnc2[nH]c(-c3ccccc3)nc12] | 2.2 ± 0.0 | ND |
| EH-93 | 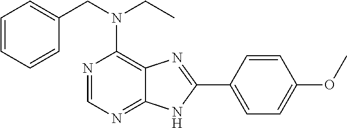 [SMILES: CCN(Cc1ccccc1)c1ncnc2[nH]c(-c3ccc(OC)cc3)nc12] | >50 | ND |

TABLE 18
Azaindol

| | | CSF1R | MAPK |
|---|---|---|---|
| Comp. | Structure | (IC₅₀, nM) | (fold) |
| CJ1_78 (comparative) | 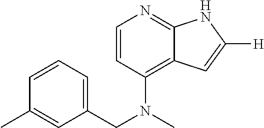 [SMILES: Cc1cccc(CN(C)c2ccnc3[nH]ccc23)c1] | >500 | ND |

### Embodiments

A1. A compound of formula (I)

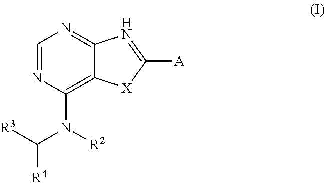

wherein:
   X is N or CH;
   A is a 6-membered hydrocarbyl or heterocyclic ring which may be optionally substituted with at least one R¹ group;
   each R¹ is independently selected from halogen, hydroxyl, —OCF₃, —CF₃, —CF₂H, —OCF₂H, CH(CF₃)OH, —O—C₁₋₆-alkyl, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, C(CH₃)₂—CH₂OH, C(CH₃)₂OH, —NH₂, NHR, —NR₂, —NO₂, —SO₂NH₂, pyridyl (preferably ortho-pyridyl), cyclopropyl or cyclobutyl substituted with an —OH group, C₁-C₁₀ aliphatic hydrocarbyl group comprising at least one heteroatom selected from 0, or N, a —O—C₁-C₁₀ aliphatic hydrocarbyl group containing at least one heteroatom selected from O or N, or a —[CH₂]ₘ-heterocycle wherein said heterocycle is optionally substituted with ═O;
   each m is 0 or 1;
   R² is methyl or ethyl, or deuterated or partially deuterated methyl or ethyl; or R² and R⁴ are linked so as to form a 5- or 6-membered ring;
   R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or when R² and R⁴ are linked so as to form a 5- or 6-membered ring, R³ can also be H or a C₁-C₄ alkyl;
   each R is C₁-C₆ alkyl;
   R⁴ is hydrogen, deuterium, C₁-C₄ alkyl, C₁-C₄ alkyl substituted with a hydroxyl group, or ═O; or R² and R⁴ are linked so as to form a 5- or 6-membered ring;
or a pharmaceutically acceptable salt or solvate thereof.

A2. A compound as defined in embodiment A1 of formula (II):

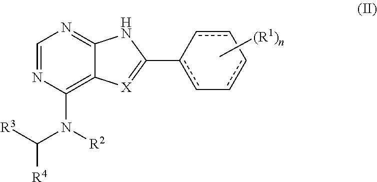

wherein:
   X, R¹, R², R³, R⁴ are as hereinbefore defined;
   each [symbol] independently represents a single or double bond;
   n is an integer between 0 and 3;
or a pharmaceutically acceptable salt or solvate thereof.

A3. The compound of any preceding embodiment wherein A is substituted with 1 or 2 R¹ groups.

A4. The compound of any preceding embodiment wherein X is CH.

A5. The compound of any preceding embodiment wherein, for R¹, said C₁-C₁₀ aliphatic hydrocarbyl group containing at least one heteroatom selected from O and N is a C₁-C₁₀, preferably C₁-C₈, preferably C₁—C aliphatic hydrocarbyl group containing at least one group selected from ether, carboxylic acid, ester, alcohol, primary amine, secondary amine, tertiary amine, amide.

A6. The compound of any preceding embodiment, wherein A is a phenyl ring optionally substituted with at least one group R¹ group.

A7. The compound of any preceding embodiment wherein R² is methyl.

A8. The compound of any preceding embodiment wherein, for R³,
said 5- or 6-membered hydrocarbyl ring is an aliphatic or aromatic hydrocarbyl ring preferably selected from phenyl, cyclopentyl, and cyclohexyl, and/or
said 5- or 6-membered heterocyclic ring is a non-aromatic heterocyclic ring comprising one heteroatom selected from N, O, or S, e.g. piperidine, tetrahydropyran, tetrahydrofuran, or a heteroaryl ring comprising one heteroatom selected from N, O, or S, e.g. pyridine, thiophene, furan.

A9. The compound as defined in any preceding embodiment, wherein R³ is a 5- or 6-membered hydrocarbyl or heterocyclic ring either of which may be optionally substituted with one or more groups independently selected from C₁-C₄ alkyl, hydroxyl, halogen, —CF₃, or —OR; or when R² and R⁴ are linked so as to form a 5- or 6-membered ring, R³ can also be H or a C₁-C₄ alkyl.

A10. The compound as defined in any preceding embodiment, wherein, for R³, said 5- or 6-membered hydrocarbyl or heterocyclic ring is a 5- or 6-membered hydrocarbyl or heterocyclic ring substituted with a methyl group, preferably a phenyl ring substituted with a methyl group, more preferably a phenyl ring substituted with a methyl group in the meta position.

A11. The compound of any preceding embodiment wherein R⁴ is hydrogen, deuterium, C₁-C₂ alkyl or C₁-C₂ alkyl substituted with a hydroxyl group, or wherein R² and R⁴ together form a —CH₂—CH₂—CH₂—unit to form a 5-membered ring, preferably R⁴ is hydrogen, methyl or —CH₂OH, most preferably R⁴ is hydrogen.

A12 The compound of any preceding embodiment, wherein each R¹ is independently selected from halogen, hydroxyl, —CF₃, —CF₂H, —O—C₁₋₆-alkyl, —C₁₋₆ alkyl-OH, —[C₁₋₆alkyl]ₘ—COOH, —[C₁₋₆alkyl]ₘ—COOC₁₋₆alkyl, —CO—[C₁₋₆alkyl]-COOR, —NH₂, —NO₂, —SO₂NH₂, pyridyl (preferably ortho-pyridyl), cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O.

A13. The compound of any preceding embodiment wherein R¹ is CO₂H, CO₂R, CH₂OH, CH₂CH₂OH, wherein R is C₁-C₈alkyl.

A14. The compound of any preceding embodiment, with the proviso that:
when R² and R⁴ together form a morpholinyl group, R³ is H, X is CH, and A is phenyl, then R¹ is not meta-NH₂;
   and/or
when A is phenyl, R¹ comprises N-methyl-piperazinyl, and X is CH, then R² and R⁴ together do not form N-methyl-piperazinyl.

A15. The compound of any preceding embodiment wherein the compound is of formula (VI):

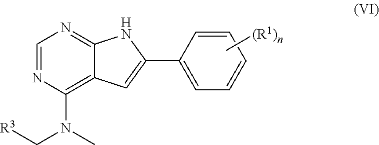

wherein R¹, n and R³ are as hereinbefore defined;
or a pharmaceutically acceptable salt or solvate thereof.

A16. A pharmaceutical composition comprising a compound as defined in any of embodiments A1 to A15 and at least one excipient.

A17. A pharmaceutical composition as defined in embodiment A16 comprising at least one other chemotherapy agent.

A18. A compound as defined in any of embodiments A1 to A15 for use in the treatment or prevention of a bone disorder, neurological disease, inflammatory disorder, cancer, or eye disease.

A19. The compound for use as defined in embodiment A18, wherein
said bone disorder is osteoporosis, osteopetrosis, or osteosarcoma,
said neurological disease is Charcot-Marie-Tooth disease, Alzheimer's disease, amyotrophic lateral sclerosis, traumatic brain injury, or Hereditary diffuse leukoencephalopathy with spheroids,
said inflammatory disorder is rheumatoid arthritis or osteoarthritis;
said cancer is multiple myeloma, ovarian cancer, glioblastoma, breast cancer, malignant peripheral nerve sheath tumor; and/or
said eye disease is macular degeneration.

A20. The compound for use as defined in embodiment A18 or A19, wherein said compound is for use in the treatment of cancer and is administered in combination with other anti-cancer agents or in combination with radiotherapy.

A21. A compound as defined in any of embodiments A1 to A15 for use as a medicament.

A22. A method of treating or preventing a bone disorder, neurological disease, inflammatory disorder, cancer, or eye disease, comprising administering a compound as define in any of embodiments A1 to A15 to a subject in need thereof, optionally in combination with other chemotherapy agents or radiotherapy when administered for treating or preventing cancer.

A23. The use of a compound as defined in embodiment A1 to A15 in the manufacture of a medicament for the treatment or prevention of a bone disorder, neurological disease, inflammatory disorder, cancer or eye disease.

A24. A process for the formation of a compound as defined in any of embodiments A1 to A15, said process comprising the steps of:
reacting a compound of formula (IV):

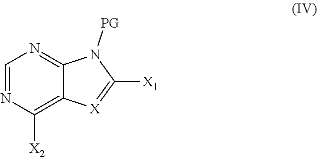

with
i) a compound of formula (RO)₂B-A or [A-BF₃]—in the presence of a transition metal catalyst, and,
ii) a compound of formula:

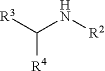

in either order (i.e. i) then ii) or ii) then i));
then
iii) removing the protecting group PG;
   wherein
X₁ and X₂ are halogen;
PG is a protecting group, preferably selected from -SEM, THP, BOC, Cbz, Fmoc, SO₂Ph, Ts, MOM, CO₂H;
R is OH, OMe, or (RO)₂ is pinacol (i.e. —O—C(CH₃)₂—C(CH₃)₂—O—) or MIDA ester (i.e. —O—CO—CH₂—N(CH₃)—CH₂—CO—O—),
A, X and R²-R⁴ are as defined in any of embodiments A1 to A15.

A25. A process for the formation of a compound as defined in any of embodiments A1-A15, said process comprising the steps of reacting a compound of formula

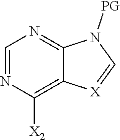

a) with a base followed by a cyclic ketone of a 6-membered hydrocarbyl or heterocyclic ring which may be optionally substituted with at least one R¹ group
b) with a compound of formula

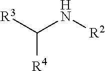

c) removing the protecting group PG;
wherein
X₂ is halogen;
PG is a protecting group, preferably selected from -SEM, THP, BOC, Cbz, Fmoc,
SO₂Ph, MOM, CO₂H, Ts;
X and R¹-R⁴ are as defined in any of embodiments A1 to 15.

1. A compound of formula (I) 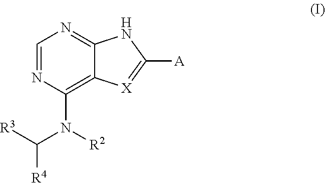
wherein:
X is CH or N;
A is a 5- or 6-membered hydrocarbyl or heterocyclic ring which may be optionally substituted with at least one R¹ group;
each R¹ is independently selected from halogen, hydroxyl, —OCF₃, —CF₃, —CF₂H, —OCF₂H, CH(CF₃)OH, —C₁₋₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —[C₁₋₆alkyl]ₘ—COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, —C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, —NH₂, NHR, —NR₂, —NO₂, —SO₂NH₂, —P(O)R₂, —SO₂R, pyridyl, cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O or —OH;
each m is 0 or 1;
R² is methyl or ethyl, or deuterated or partially deuterated methyl or ethyl; or R² and R⁴ are linked so as to form a 5- or 6-membered ring;
R³ is a 5- or 6-membered hydrocarbyl or heterocyclic ring either of which may be optionally substituted with one or more groups independently selected from C₁-C₄ alkyl, hydroxyl, halogen, —CF₃, —CF₂H, —NR₂, —NHCOR, —CO₂H, —CO₂R, or —OR; or
when R² and R⁴ are linked so as to form a 5- or 6-membered ring, R³ can also be H or a C₁-C₄ alkyl;
each R is C₁-C₆ alkyl;
R⁴ is hydrogen, deuterium, C₁-C₄ alkyl, C₁-C₄ alkyl substituted with a hydroxyl group, or ═O; or R² and R⁴ are linked so as to form a 5- or 6-membered ring;
or a pharmaceutically acceptable salt or solvate thereof.

2. The compound as claimed in claim 1, wherein the compound is of formula (II): 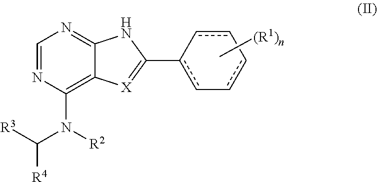
wherein:
each [symbol] independently represents a single or double bond; and
n is an integer between 0 and 3;
or a pharmaceutically acceptable salt or solvate thereof.

3. The compound of claim 1, wherein A is substituted with 1 or 2 R¹ groups.

4. The compound of claim 1, wherein X is CH.

5. The compound of claim 1, wherein, for R¹, said C₁-C₁₀ aliphatic hydrocarbyl group containing at least one heteroatom selected from 0 and N is a C₁-C₁₀.

6. The compound of claim 1, wherein A is a phenyl ring optionally substituted with at least one group R group.

7. The compound of claim 1, wherein R² is methyl.

8. The compound of claim 1, wherein, for R³,
said 5- or 6-membered hydrocarbyl ring is an aliphatic or aromatic hydrocarbyl ring, and/or
said 5- or 6-membered heterocyclic ring is a non-aromatic heterocyclic ring comprising one heteroatom selected from N, O, or S,
or a heteroaryl ring comprising one heteroatom selected from N, O, or S.

9. (canceled)

10. (canceled)

11. The compound of claim 1, wherein R⁴ is hydrogen, deuterium, C₁-C₂ alkyl or C₁-C₂ alkyl substituted with a hydroxyl group, or wherein R² and R⁴ together form a -CH2-CH2-CH2- unit to form a 5-membered ring.

12. The compound of claim 1, wherein each R¹ is independently selected from halogen, hydroxyl, —CF₃, —CF₂H, —O—C₁₋₆-alkyl, —C₁₋₆alkyl-OH, —[C₁-6alkyl]ₘ—COOH, —[C₁₋₆alkyl]ₘ—COOC₁₋₆alkyl, —CO—[C₁₋₆alkyl]-COOR, —NH₂, —NO₂, —SO₂NH₂, pyridyl, cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂]ₘ-heterocycle wherein said heterocycle is optionally substituted with ═O.

13. The compound of claim 1, wherein R¹ is CO₂H, CO₂R, CH₂OH, CH₂CH₂OH, wherein R is C₁-C₆ alkyl.

14. The compound of claim 1, with the proviso that:
when R² and R⁴ together form a morpholinyl group, R³ is H, X is CH, and A is phenyl, then R¹ is not meta-NH₂;
and/or
when A is phenyl, R¹ comprises N-methyl-piperazinyl, and X is CH, then R² and R⁴ together do not form N-methyl-piperazinyl.

15. The compound of claim 1, wherein the compound is of formula (VI): 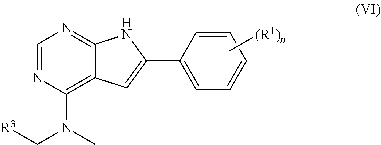
or a pharmaceutically acceptable salt or solvate thereof.

16. The compound of claim 1, wherein:
when X is N and R² and R⁴ are linked so as to form a 5- or 6-membered ring, then A is not pyridyl, morpholinyl or piperidinyl;
when X is N and R² and R⁴ are linked so as to form a 5- or 6-membered ring, then R₃ is not H;
when X is N and R² and R⁴ are linked so as to form a 5- or 6-membered ring, then A is phenyl which may be optionally substituted with at least one R¹ group:
when X is CH and R² and R⁴ are linked so as to form a 5- or 6-membered ring, then A is not phenyl substituted with —NH₂, —NO₂, [C₁₋₆-alkyl]—COOH or Br;
when X is CH and R² and R⁴ are linked so as to form a 5- or 6-membered ring, then R² and R⁴ do not form a piperidinyl ring;
when X is N and R² and R⁴ are linked so as to form a 5- or 6-membered ring, then R³ is not H;
when X is N and R² and R⁴ together form a morpholinyl or pyrrolidinyl ring, then A is not pyridyl;
when X is N and R² and R⁴ together form a piperazinyl ring, then A is not morpholinyl;
when X is N and R² and R⁴ together form a piperazinyl ring, then A is not pyridyl;
when X═CH and R² and R⁴ form a 5- or 6-membered ring, then R¹ is not —NH₂, or A is not phenyl with para-NH₂;
when X═CH and R² and R⁴ form a 6-membered ring, or when R² and R⁴ form a 6-membered ring, then R¹ is not —NH₂ or A is not phenyl with para-NH₂;
when R² and R⁴ form a 5- or 6-membered ring, then A is not phenyl and R¹ is not —NH₂;
when R² and R⁴ together form a morpholinyl group and R³ is H, then A is not a 6-membered ring substituted with para-NH₂ or —CH₂COOH:
when X═CH and R² and R⁴ together form a 6-membered ring, R³ is H, then A is not a phenyl substituted with para-Br;
when X═CH and R² and R⁴ form piperazinyl, then A is not unsubstituted phenyl and/or R² and R⁴ do not form piperazinyl;
when X═CH and R² and R⁴ together form an imidazole ring, then A is not phenyl substituted with para-NH₂;
when X═CH and R² and R⁴ together form a 5-membered ring, then A is not a phenyl substituted with meta-NO₂:
when X═CH and R² and R⁴ are linked so as to form a 5- or 6-membered ring, then R² and R⁴ do not form a piperidinyl ring:
when X═CH and R² and R⁴ are linked so as to form a 5- or 6-membered ring and A is unsubstituted phenyl, then R² and R⁴ do not form a piperidinyl ring;
when X is CH, R² and R⁴ are linked so as to form a 5- or 6-membered ring and A is phenyl which may be optionally substituted with at least one R¹ group, then each R¹ is independently selected from Cl, F, I, hydroxyl, —OCF₃, —CF₃, —CF₂H, —OCF₂H, CH(CF₃)OH, —C₁-C₆-alkyl, —O—C₁₋₆-alkyl, —O—(C₁-C₆ alkyl)-CH₂F, —O—(C₁-C₆ alkyl)-CHF₂, —O—(C₁-C₆ alkyl)-CF₃, —C₁₋₆alkyl-OH, —COOH, —[C₁₋₆alkyl]ₘ—COOC₁₋₆alkyl, —[C₁₋₆alkyl]ₘ—OCOC₁₋₆alkyl, —OCOR, —CO—[C₁₋₆alkyl]—COOH, —CO—[C₁₋₆alkyl]-COOR, C(CH₃)₂—CH₂OH, —C(OH)₂(C₁₋₆alkyl-OH), —C(OH)(C₁₋₆alkyl-OH)₂, —C(C₁₋₆alkyl-OH)₃, C(CH₃)₂OH, NHR, —NR₂, —SO₂NH₂, —P(O)R², —SO₂R, pyridyl, cyclopropyl or cyclobutyl substituted with an —OH group, C₁-C₁₀ aliphatic hydrocarbyl group comprising at least one heteroatom selected from O, or N, a —O—C₁-C₁₀ aliphatic hydrocarbyl group containing at least one heteroatom selected from O or N, or a —[CH₂₁m-heterocycle wherein said heterocycle is optionally substituted with ═O or —OH:
when R² and R⁴ are linked so as to form a 5- or 6-membered ring, R² and R⁴ are linked so as to form a pyrrolidinyl or morpholinyl ring;
or a combination thereof.

17.-36. (canceled)

37. The compound of claim 1, wherein
when X is N and R² and R⁴ are linked so as to form a 5- or 6-membered ring, then the compound of formula (I) is selected from

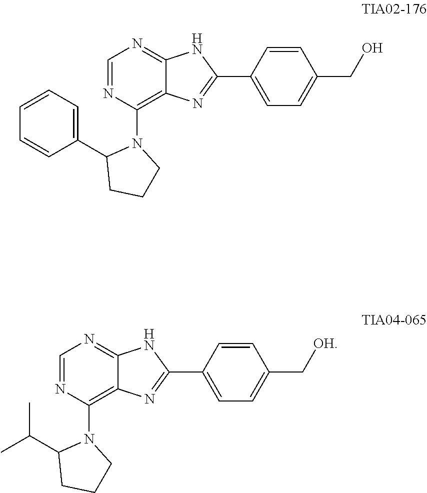 [SMILES: OCc1ccc(-c2nc3c(N4CCCC4c4ccccc4)ncnc3[nH]2)cc1 ; CC(C)C1CCCN1c1ncnc2[nH]c(-c3ccc(CO)cc3)nc12]

38. The compound of claim 1, wherein
when X is CH and R² and R⁴ are linked so as to form a 5- or 6-membered ring, then the compound of formula (I) is selected from:

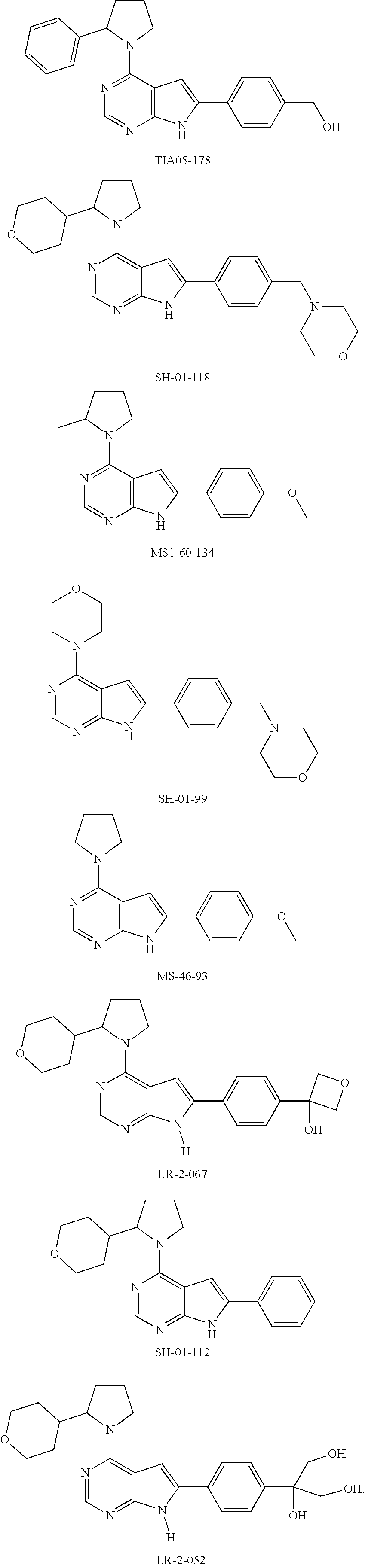

39. A pharmaceutical composition comprising the compound as claimed in claim 1 and at least one excipient, and optionally further comprising at least one other chemotherapy agent.

40.-44. (canceled)

45. A method of treating or preventing a bone disorder, neurological disease, inflammatory disorder, cancer, or eye disease, comprising administering the compound as claimed in claim 1 to a subject in need thereof, optionally in combination with other chemotherapy agents or radiotherapy when administered for treating or preventing cancer.

46. (canceled)

47. A process for the formation of the compound as claimed in claim 1, said process comprising the steps of:
reacting a compound of formula (IV): 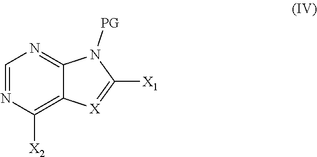
with
i) a compound of formula (RO)₂B-A or [A-BF3]⁻ in the presence of a transition metal catalyst, and,
ii) a compound of formula: 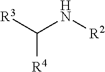
in either order
then
iii) removing the protecting group PG;
wherein
X₁ and X₂ are halogen;
PG is a protecting group; and
R is OH, OMe, or (RO)₂ is pinacol.

48. A process for the formation of the compound as claimed in claim 1, said process comprising the steps of
reacting a compound of formula 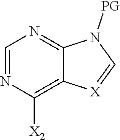
a) with a base followed by a cyclic ketone of a 6-membered hydrocarbyl or heterocyclic ring which may be optionally substituted with at least one R group
b) with a compound of formula 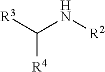
c) removing the protecting group PG;
wherein
X₂ is halogen; and
PG is a protecting group.
